(19) United States
(12) Patent Application Publication
(10) Pub. No.: US 2022/0304291 A1
(43) Pub. Date: Sep. 29, 2022

(54) NOVEL METHODS FOR REARING AND CONTROLLED RELEASE OF PREDATORY MITES

(71) Applicant: Bio-Bee SDE Eliyahu Ltd., Emmek H'maayanot (IL)

(72) Inventors: Arnon TABIC, Lower Galilee (IL); Tom KATZ, Degania B (IL); Amir GROSMAN, Amsterdam (NL); Shimon STEINBERG, Timrat (IL)

(21) Appl. No.: 17/639,800

(22) PCT Filed: Sep. 3, 2019
(86) PCT No.: PCT/IL2019/050982

§ 371 Date: Mar. 2, 2022

(51) Int. Cl. A01K 67/033; A01G 13/00

(52) U.S. Cl. CPC A01K 67/033; A01G 13/00

(57) ABSTRACT

The present invention discloses predatory mite population comprising Phytoseiulus predatory individuals, wherein at least 10% of female individuals of the population is capable of reproduction on a non-tetranychid arthropod prey, preferably on an immobilized non-tetranychid arthropod prey, such as on a non-phytophagous prey, preferably on an Astigmatid prey, most preferably on an immobilized Astigmatid prey, such as an immobilized Astigmatid prey, in particular a Carpoglyhus prey, having immobilized life stages comprising immobilized eggs. Methods for rearing the same are further disclosed.

### BACKGROUND OF THE INVENTION

### Field of the Invention

The present invention relates to the field of biological control agents for crop protection, and more particularly to novel means and methods for rearing biological control agents against plant pests.

### Background Art

The use of arthropods (insects and mites) as Biological Control Agents (BCA) is an expanding field with many advantages over chemical pest control. Arthropod BCA's are able to naturally control other arthropod species that act as pests on the crop.

Phytoseiulus is a genus of mites in the Phytoseiidae family. This predatory mite is the most frequently used to control two-spotted spider mites in greenhouses and outdoor crops grown in mild environments. A Phytoseiulus mite can consume up to seven adult spider mites or several dozens of their eggs in a day. A well-fed female lays about 50 eggs in her lifetime. The genus Phytoseiulus contains four known species, namely: P. persimilis, P. longipes, P. macropilis and P. fragariae (Chant and McMurtry 2006). All species of the genus Phytoseiulus are considered type 1 predators, i.e. highly specific to a diet consisting of spider mites, preferably of the genus Tetranychus (McMurtry and Croft 1997). The most frequently used species of this genus for biological control of spider mites is Phytoseiulus persimilis.

Phytoseiulus persimilis (P. persimilis) adults are bright reddish-orange in color, with long legs and pear-shaped bodies (about 0.5 mm long).

P. persimilis is considered as a specialist for spider mites (mites of the family Tetranychidae) which are phytophagous mites (Belle and Sabelis 1985, Gerson et al. 2003). Gerson et al. 2003 specifically indicate that “members of the genus Phytoseiulus live and place their eggs almost exclusively within the webbed colonies of Tetranychus spp”. It is further noted in Gerson et al. 2003 that “the specificity of P. persimilis for spider mite prey can be a disadvantage if other predators are present on the same plants”.

It was found that P. persimilis might develop and possibly reproduce on another phytophagous (plant feeding) mite, Steneotarsonemus pallidus of the family Tarsonemidae (Simmonds, S. P., 1970)

From a commercial point of view, a significant disadvantage of producing a predatory mite that exclusively feeds on phytophagous mites, such as spider mites, is that it requires rearing prey mites on plants, which has a high cost.

Walzer and Schausberger, 1999, examined intra- and interspecific predation of adult females and immature stages of the more generalist Neoseiulus californicus and the specialist Phytoseiulus persimilis. It was reported that adult females and immatures of both predators exhibited higher predation rates on larvae than on eggs and protonymphs. It was found that predation on P. persimilis by N. californicus was more severe than vice versa. P. persimilis was reported to have higher predation rates on conspecifics than heterospecifics and was more prone to cannibalism than N. californicus. In addition, it was reported that when provided with phytoseiid prey, P. persimilis suffered higher mortality than N. californicus.

Walzer and Schausberger, 1999 further teach that females of P. persimilis were not able to sustain oviposition, irrespective of con- or heterospecific prey. Furthermore, mortality of P. persimilis immatures was less when feeding on conspecific vs. heterospecific larvae. These authors concluded that for P. persimilis, neither heteronor conspecific prey provides sufficient nourishment for sustained reproduction. This is supported by Yao and Chant (1989), reporting that P. persimilis did not produce eggs when either cannibalizing or preying upon immatures of Iphyseius degenerans. There were only two females in this study that laid a single egg when cannibalizing conspecific.

In summary, it was found that P. persimilis was able to develop on juvenile predatory mites Neoseiulus californicus and Iphyseius degenerans of the family Phytoseiidae. However, it did not lay eggs when feeding on these prey mites. On the other hand, when the predatory mite N. californicus and I. degenerans fed on P. persimilis, they did lay eggs (Yao and Chant, 1989). This demonstrates the narrow dietary range of P. persimilis in contrast to other mites of the same family. P. persimilis might also develop in a cannibalistic manner, feeding on younger stages of its own. When feeding this way, there were rare cases of oviposition (Walzer and Schausberger, 1999; Yao and Chant, 1989). In all cases where the Phytoseiidae mites were used as prey, the latter was fed with spider mites, which are grown on plants and therefore involves high costs.

P. persimilis was further found to develop on thrips (a phytophagous insect) larvae, but it did not lay eggs on this diet (Walzer 2004). This is in contrast to the predatory mite N. califonicus that was able to reproduce on this prey (Walzer 2004). It should be emphasized that in this study, a high rate of mortality was reported during juvenile development.

U.S. Pat. No. 9,781,937 and EP patent 2612551 disclose a mite composition comprising predatory mite species selected from Mesostigmatid mite species or Prostigmatid mite species and a food source for the predatory mite species comprising Astigmatid mite species. It is further disclosed in these publications that at least a fraction of the Astigmatid individuals is immobilized and that the immobilized Astigmatid individuals are contacted with a fungus reducing agent comprising a fungus reducing mite population selected from a mycophagous mite species or an antifungal exudates producing mite species.

U.S. Pat. No. 7,947,269 teaches a mite composition comprising a rearing population of a phytoseiid predatory mite species and a factitious host population comprising at least one species selected from the family of the Carpoglyphidae.

U.S. Pat. No. 8,097,248 discloses a mite composition comprising a rearing population of the phytoseiid predatory mite species Amblyseius swirskii, a factitious host population comprising at least one Astigmatid mite species selected from the group consisting of: i) Carpoglyphidae, ii) Pyroglyphidae, and iii) Glyciophagidae.

U.S. Pat. No. 8,733,283 discloses a method for rearing predatory mites by providing a food source for prey mites that comprises dextrose; rearing Thyreophagus entomophagus prey mites on said food source; providing predatory mites that feed on Thyreophagus entomophagus in a starting ratio of predatory mites to prey mites from 1:10 to 1:100, and rearing the predatory mites on said prey mites, to create a breeding population.

U.S. Pat. No. 8,733,283 and EP2048941 patents teach that Phytoseiulus persimilis can be only raised on spider mite diet. They report that P. persimilis is an obligate spider mite predator and cannot survive on alternate food sources such as pollen. It is emphasized in these publications that survival tends to be poor if prey is in short supply.

EP2380436 discloses a mite composition comprising a rearing population of a phytoseiid predatory mite species and a population of at least one species from the order Astigmata characterized in that the population of the species from the order Astigmata is not alive.

WO2007075081 discloses mite composition comprising a rearing population of a phytoseiid predatory mite species and a factitious host population characterised in that the factitious host population comprises at least one species selected from the family of the Glyciphagidae. When referring to the phytoseiid mite Phytoseiulus persimilis, it is indicated that spider mites (Tetranychus urticae) is the best prey.

None of the above patent documents discloses or teaches successfully rearing the important predatory mite Phytoseiulus persimilis on mites of the order Astigmata, in any form or developmental stage. On the contrary, all the above patent documents and scientific publications report that P. persimilis is an obligate spider mite predator and it cannot survive on alternate food sources. Therefore, an entomologist/acarologist would not consider P. persimilis as a typical generalist species of the Phytoseiidae family or the Amblyseiinae subfamily but rather a highly specific species.

In view of the above, there is a long felt need for effective and efficient mass rearing of Phytoseiulus persimilis for biological control of crop pests.

### SUMMARY OF THE INVENTION

The present invention relates to the field of insect control and more specifically to a system and method for rearing biological control agents against plant pests.

It is one object of the present invention to disclose predatory mite population comprising Phytoseiulus predatory individuals, wherein at least 10% of female individuals of the population is capable of reproduction on a non-tetranychid arthropod prey, preferably on an immobilized non-tetranychid arthropod prey, such as on a non-phytophagous prey, preferably on an Astigmatid prey, most preferably on an immobilized Astigmatid prey, such as an immobilized Astigmatid prey, in particular a Carpoglyhus prey, having immobilized life stages comprising immobilized eggs.

It is a further object of the present invention to disclose the predatory mite population as defined above, wherein at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of female individuals of the population are capable of reproducing on a non-tetranychid arthropod prey, preferably on an immobilized non-tetranychid arthropod prey, such as on a non-phytophagous prey, preferably on an Astigmatid prey, most preferably on an immobilized Astigmatid prey,such as an immobilized Astigmatid prey having immobilized life stages comprising immobilized eggs.

It is a further object of the present invention to disclose the predatory mite population as defined in any of the above, wherein the at least 10% of female individuals of the population are capable of oviposition on the non-Tetranychid arthropod prey, preferably on immobilized Astigmatid prey having immobilized life stages comprising immobilized eggs.

It is a further object of the present invention to disclose the predatory mite population as defined in any of the above, wherein the population has a daily oviposition rate of at least 0.50, such as ≥0.60, ≥0.65, ≥0.70, ≥0.75, ≥0.80, ≥0.90, ≥0.95, ≥1.00, ≥1.05, ≥1.10, ≥1.15, ≥1.20, ≥1.25, ≥1.30, ≥1.35, ≥1.40, ≥1.45, ≥1.50, ≥1.55, ≥1.60, ≥1.65, ≥1.70, ≥1.75, ≥1.80, ≥1.85, ≥1.90, ≥1.95 or at least 2.00 eggs/day/female on the non-Tetranychid arthropod prey, preferably on immobilized Astigmatid prey having immobilized life stages comprising immobilized eggs.

It is a further object of the present invention to disclose the predatory mite population as defined in any of the above, wherein the population has a daily oviposition rate of at least 0.55, such as ≥0.60, ≥0.65, ≥0.70, ≥0.75, ≥0.80, ≥0.90, ≥0.95, ≥1.00, ≥1.05, ≥1.10, ≥1.15, ≥1.20, ≥1.25, ≥1.30, ≥1.35, ≥1.40, ≥1.45, ≥1.50, ≥1.55, ≥1.60, ≥1.65, ≥1.70, ≥1.75, ≥1.80, ≥1.85, ≥1.90, ≥1.95, or ≥2.00 eggs/day/female, when using the non-tetranychid arthropod prey as the sole food source .

It is a further object of the present invention to disclose the predatory mite population as defined in any of the above, wherein the at least 10% of female individuals are capable of completing a full ontogenetic cycle, when using the non-tetranychid arthropod prey as the sole food source.

It is a further object of the present invention to disclose the predatory mite population as defined in any of the above, wherein the population is characterized by a juvenile and/or female survival rate of at least 40%, preferably at least 45%, 50%, 60%, 65%, 70%, 75%, 80%, 85%, 90%, or at least 95% on the non-Tetranychid prey.

It is a further object of the present invention to disclose the predatory mite population as defined in any of the above, wherein the at least 10% of female individuals of the population are characterized by capability to produce female offspring in a number of subsequent generations, wherein the number of subsequent generations is at least 1, such as at least 2, such as in at least 3, 4, 5, 6, 7, 8, 9 at least 10 generations.

It is a further object of the present invention to disclose the predatory mite population as defined in any of the above, wherein the population is characterized by a daily reproduction rate in the range of about 1.10 -1.40, such as 1.15-1.40, 1.20-1.40, 1.25-1.40, 1.30-1.40, or 1.10-1.35, 1.10-1.30, 1.10-1.25, 1.10-1.20 on the non-Tetranychid arthropod prey, preferably on immobilized Astigmatid prey having immobilized life stages comprising immobilized eggs.

It is a further object of the present invention to disclose the predatory mite population as defined in any of the above, wherein female individuals have predatory behavior towards individuals of a Tetranychid species, preferably predatory behavior characterized by a daily oviposition rate of at least 10, preferably at least 15, more preferably at least 19 eggs per female per 5 days.

It is a further object of the present invention to disclose the predatory mite population as defined in any of the above, wherein the population has an increased reproduction rate as compared to a control Phytoseiulus predatory population of the same species comprising a fraction of female individuals capable of reproduction on immobilized Astigmatid prey having immobilized life stages comprising immobilized eggs, lower than 10%.

It is a further object of the present invention to disclose Predatory mite population comprising Phytoseiulus predatory individuals, wherein the population is characterized by a daily oviposition rate of at least 0.55 eggs/day/female, such as ≥0.60, ≥0.65, ≥0.70, ≥0.75, ≥0.80, ≥0.90, ≥0.95, ≥1.00,≥1.05, ≥1.10, ≥1.15, ≥1.20, ≥1.25, ≥1.30, ≥1.35, ≥1.40, ≥1.45, ≥1.50, ≥1.55, ≥1.60, ≥1.65, ≥1.70, ≥1.75, ≥1.80, ≥1.85, ≥1.90, ≥1.95, or ≥2.00 eggs/day/female, while preying on non-tetranychid arthropod prey, preferably on an immobilized non-tetranychid arthropod prey, such as on a non-phytophagous prey, preferably on an Astigmatid prey, most preferably on an immobilized Astigmatid prey, such as an immobilized Astigmatid prey having immobilized life stages comprising immobilized eggs.

It is a further object of the present invention to disclose the predatory mite population as defined in any of the above, wherein the population is characterized by a juvenile and/or female survival rate of at least 40% on the non-Tetranychid prey.

It is a further object of the present invention to disclose the predatory mite population as defined in any of the above, wherein at least 10% of female individuals of the population are characterized by capability to produce female offspring in a number of subsequent generations, wherein the number of subsequent generations is at least 1, such as at least 2, such as in at least 3, 4, 5, 6, 7, 8, 9 at least 10 generations.

It is a further object of the present invention to disclose the predatory mite population as defined in any of the above, wherein the population is characterized by a daily reproduction rate in the range of about 1.10-1.40, such as 1.15-1.40, 1.20-1.40, 1.25-1.40, 1.30-1.40, or 1.10-1.35, 1.10-1.30, 1.10-1.25, 1.10-1.20.

It is a further object of the present invention to disclose the predatory mite population as defined in any of the above, wherein at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of female individuals of the population are capable of reproducing on a non-tetranychid arthropod prey, preferably on an immobilized non-tetranychid arthropod prey, such as on a non-phytophagous prey, preferably on an Astigmatid prey, most preferably on an immobilized Astigmatid prey, such as an immobilized Astigmatid prey having immobilized life stages comprising immobilized eggs.

It is a further object of the present invention to disclose predatory mite population comprising Phytoseiulus predatory individuals, wherein the population is characterized by improved reproduction on non-tetranychid arthropod prey, preferably on an immobilized non-tetranychid arthropod prey, such as on a non-phytophagous prey, preferably on an Astigmatid prey, most preferably on an immobilized Astigmatid prey, such as an immobilized Astigmatid prey having immobilized life stages comprising immobilized eggs, as compared to a control Phytoseiulus predatory population of the same species comprising a fraction of female individuals capable of reproduction on immobilized Astigmatid prey having immobilized life stages comprising immobilized eggs, lower than 10%.

It is a further object of the present invention to disclose the predatory mite population as defined in any of the above, wherein the improved reproduction on a non-tetranychid arthropod prey is characterized by at least one of: increased daily reproduction rate, increased daily oviposition rate, increased survival rate, increased percentage of female individuals reproducing on said prey, and improved predatory behavior towards a Tetranychidae.

It is a further object of the present invention to disclose the predatory mite population as defined in any of the above, wherein predatory individuals are from a species selected from Phytoseiulus fragariae, Phytoseiulus longipes, Phytoseiulus macropilis, Phytoseiulus persimilis and Phytoseiulus robertsi.

It is a further object of the present invention to disclose the predatory mite population as defined in any of the above, wherein the reproduction on a non-tertranychid prey is reproduction on an Astigmatid mite species selected from:

i) Carpoglyphidae such as from the genus Carpoglyphus e.g. Carpoglyphus actis;

ii) Pyroglyphidae such as from the genus Dermatophagoides e.g. Dermatophagoides pteronysinus, Dermatophagoides farinae; from the genus Euroglyphus e.g. Euroglyphus longior, Euroglyphus maynei; from the genus Pyroglyphus e.g. Pyroglyphus africanus;

iii) Glycyphagidae such as from the subfamily Ctenoglyphinae, such as from the genus Diamesoglyphus e.g. Diamesoglyphus intermediusor from the genus Ctenoglyphus, e.g. Ctenoglyphus plumiger, Ctenoglyphus canestrinii, Ctenoglyphus palmifer; the subfamily Glycyphaginae, such as from the genus Blomia, e.g. Blomia freemani or from the genus Glycyphagus, e.g. Glycyphagus ornatus, Glycyphagus bicaudatus, Glycyphagus privatus, Glycyphagus domesticus, or from the genus Lepidoglyphus e.g. Lepidoglyphus michaeli, Lepidoglyphus fustifer, Lepidoglyphus destructor, or from the genus Austroglycyphagus, e.g. Austroglycyphagus geniculatus; from the subfamily Aeroglyphinae, such as from the genus Aeroglyphus, e.g. Aeroglyphus robustus; from the subfamily Labidophorinae, such as from the genus Gohieria, e.g. Gohieria fusca; or from the subfamily Nycteriglyphinae such as from the genus Coproglyphus, e.g. Coproglyphus stammeri or from the subfamily Chortoglyphidae, such as the genus Chortoglyphus e.g. Chortoglyphus arcuatus and more preferably is selected from the subfamily Glycyphaginae, more preferably is selected from the genus Glycyphagus or the genus Lepidoglyphus most preferably selected from Glycyphagus domesticus or Lepidoglyphus destructor;

iv) Acaridae such as from the genus Tyrophagus e.g. Tyrophagus putrescentiae, Tyrophagus tropicus, from the genus Acarus e.g. Acarus siro, Acarus farris, Acarus gracilis; from the genus Lardoglyphus e.g. Lardoglyphus konoi, from the genus Thyreophagus, such as Thyreophagus entomophagus; from the genus Aleuroglyphus, e.g. Aleuroglyphus ovatus;

v) Suidasiidae such as from the genus Suidasia, such as Suidasia nesbiti, Suidasia pontifica or Suidasia medanensis.

It is a further object of the present invention to disclose mite composition comprising a predatory mite population as defined in any of the above together with a carrier material, such as a carrier material selected from sawdust, wheat bran, buckwheat husks, rice husks or millet husks, or comprising a mixture thereof, preferably a carrier having carrier elements comprising mite shelters.

It is a further object of the present invention to disclose mite composition as defined above, comprising a food source for the Phytoseiulus predatory individuals, wherein the food source comprises a non-Tetranychid arthropod prey, preferably an immobilized non-tetranychid arthropod prey, such as a non-phytophagous prey, preferably an Astigmatid prey, most preferably an immobilized Astigmatid prey, such as an immobilized Astigmatid prey, in particular a Carpoglyhus species, having immobilized life stages comprising immobilized eggs.

It is a further object of the present invention to disclose use of a non-Tetranychid arthropod species, preferably an immobilized non-Tetranychid arthropod species, such as a non-phytophagous prey, preferably an Astigmatid species, most preferably an immobilized Astigmatid species, such as most preferably an immobilized Astigmatid species, in particular a Carpoglyhus species, having immobilized life stages comprising immobilized eggs, as a food source, preferably as a rearing prey, for a predatory mite population of Phytoseiulus predatory individuals as defined in any of the above.

It is a further object of the present invention to disclose the use as defined in any of the above, wherein said use comprises releasing individuals of a non-Tetranychid arthropod species, preferably an immobilized non-Tetranychid arthropod species, such as a non-phytophagous prey, preferably an Astigmatid species, most preferably an immobilized Astigmatid species, such as most preferably an immobilized Astigmatid species, in particular a Carpoglyhus species, having immobilized life stages comprising immobilized eggs, preferably the use comprises releasing the non-Tetranychid arthropod species using a device comprising an exit for mobile life stages of the non-Tetranychid arthropod mite species, preferably an exits suitable for providing a sustained release of a number of mobile life stages .

It is a further object of the present invention to disclose the use as defined in any of the above, wherein said use comprises applying individuals of a non-Tetranychid arthropod species to a target plant, preferably an immobilized non-Tetranychid arthropod species, such as a non-phytophagous prey, preferably an Astigmatid species, most preferably an immobilized Astigmatid species, such as most preferably an immobilized Astigmatid species, in particular a Carpoglyhus species, having immobilized life stages comprising immobilized eggs, or a mixture of immobilized life stages comprising eggs and mobile stages of the non-Tetranychid arthropod species.

It is a further object of the present invention to disclose a device for releasing individuals of a Phytoseiulus predatory mite species, said device comprising a container holding a predatory mite population as defined in any of the above, preferably in a compositionas defined in any of the above, wherein the device comprises an exit for mobile life stages of the Phytoseiulus predatory mite species, preferably an exits suitable for providing a sustained release of a number of mobile life stages.

It is a further object of the present invention to disclose use of a predatory mite population as defined in any of the above, or a mite composition as defined in any of the above, preferably in a device as defined in any of the above, for crop protection.

It is a further object of the present invention to disclose a method for rearing Phytoseiulus predatory individuals, said method comprising providing a predatory mite population as fefined in any of the above, preferably in a composition as defined in any of the above, and allowing the Phytoseiulus predatory individuals to prey on the non-Tetranychid arthropod prey.

It is a further object of the present invention to disclose a method for obtaining a predatory mite population as defined in any of the above, said method comprising the steps of:

(a) providing a rearing population of a predatory mite species selected from the genus Phytoseiulus, said rearing population comprising individuals of the Phytoseiulus species preferably together with a suitable food source for the Phytoseiulus individuals, said food source comprising a prey species selected from the Tetranychidae;

(b) providing a preselected non-Tetranychid arthropod species, preferably an Astigmatid mite species, most preferably an immobilized Astigmatid mite species having immobilized life stages comprising immobilized eggs;

(c) providing a the preselected non-Tetranychid arthropod pecies to the Phytoseiulus individuals as a food source;

(d) selecting Phytoseiulus individuals that are capable of reproduction while using the preselected non-Tetranychid arthropod individuals as a food source;

(e) rearing selected Phytoseiulus individuals on a food source comprising the preselected non-Tetranychid arthropod species;

(f) optionally, alternatingly rearing selected Phytoseiulus individuals in a sequence of:
rearing for at least 2, such as between 5 and 50, generations while using a food source comprising the preselected non-Tetranychid arthropod species;
rearing for at least 2, such as between 5 and 50, generations while using a food source comprising the prey species selected from the Tetranychidae.

It is a further object of the present invention to disclose the method as defined in any of the above, wherein the method further comprises steps of

a. separating eggs from the preselected non-Tetranychid arthropod species;

b. mixing the separated eggs with a carrier material, such as a carrier material selected from sawdust, wheat bran, buckwheat husks, rice husks or millet husks, or comprising a mixture thereof and water, so as to coat the carrier material with layer of eggs;

c.freezing the mixture; and

d. rearing the Phytoseiulus individuals on the mixture as a food source.

It is a further object of the present invention to disclose method as defined in any of the above, wherein the provided rearing population is a population composed of a number of sub-populations, wherein said sub-populations are from distinct sources, such as from distinct production populations and/or from natural populations isolated from distinct geographical locations.

It is a further object of the present invention to disclose the method as defined in any of the above, wherein the provided rearing population comprises at least 100 individuals, such as between 200 and 5000 individuals, preferably between 500 and 1500 individuals.

It is a further object of the present invention to disclose a method for obtaining predatory mite population capable of reproduction on a non-tertranychid arthropod prey, preferably on an immobilized non-tetranychid arthropod prey, such as on a non-phytophagous prey, preferably on an Astigmatid prey, most preferably on an immobilized Astigmatid prey, such as an immobilized Astigmatid prey having immobilized life stages comprising immobilized eggs, said method comprising the steps of:

a. providing a rearing population of a predatory mite species selected from the genus Phytoseiulus, said rearing population comprising individuals of the Phytoseiulus species, reared on a suitable food source for the Phytoseiulus individuals, said food source comprising a prey species selected from the Tetranychidae species of the genus Phytoseiulus;

b. providing a population of individuals of preselected non-Tetranychid arthropod species, preferably an Astigmatid mite species, most preferably an immobilized Astigmatid mite species having immobilized life stages comprising immobilized eggs;
c. rearing Phytoseiulus individuals on the preselected non-Tetranychid arthropod species as a food source.

It is a further object of the present invention to disclose the method qas defined in any of the above, further comprising steps of:

d. selecting Phytoseiulus individuals that are capable of reproduction while using the preselected non-Tetranychid arthropod individuals as a food source; e.rearing selected Phytoseiulus individuals on a food source comprising the preselected non-Tetranychid arthropod species;

f. optionally, alternatingly rearing selected Phytoseiulus individuals in a sequence of:
rearing for at least 2, such as between 5 and 50, generations while using a food source comprising the preselected non-Tetranychid arthropod species;
rearing for at least 2, such as between 5 and 50, generations while using a food source comprising the prey species selected from the Tetranychidae.

It is a further object of the present invention to disclose mite composition comprising a predatory mite population according to any of the claims 1-20 together with an immobilized non-Tetranychid arthropod prey, preferably an immobilized non-Tetranychid arthropod prey comprising immobilized eggs, such as an immobilized Astigmatid mite species having immobilized life stages comprising frozen eggs, wherein the eggs are coated with a carrier material, such as a carrier material selected from sawdust, wheat bran, buckwheat husks, rice husks or millet husks, or comprising a mixture thereof, preferably a carrier having carrier elements comprising mite shelters, or wherein the carrier material, such as a carrier material selected from sawdust, wheat bran, buckwheat husks, rice husks or millet husks, or comprising a mixture thereof, preferably a carrier having carrier elements comprising mite shelters is coated by the immobilized non-Tetranychid arthropod prey.

It is a further object of the present invention to disclose a device for releasing individuals of a Phytoseiulus predatory mite species, said device comprising a container holding the composition as defined in any of the above, wherein the container comprises an exit for mobile life stages of the Phytoseiulus predatory mite species, preferably an exits suitable for providing a sustained release of a number of mobile life stages.

It is a further object of the present invention to disclose a biological control composition wherein the composition comprises

a. a predatory mite population comprising individuals of at least one mite species of the genus Phytoseiulus capable of reproduction on a non-tetranychid arthropod prey, preferably on an immobilized non-tetranychid arthropod prey, such as on a non-phytophagous prey, preferably on an Astigmatid prey, most preferably on an immobilized Astigmatid prey, such as an immobilized Astigmatid prey, in particular a Carpoglyhus prey, having immobilized life stages comprising immobilized eggs; and

b. a prey mite population comprising individuals of a non-tetranychid arthropod prey, preferably an immobilized non-tetranychid arthropod prey, such as a non-phytophagous prey, preferably an Astigmatid prey, most preferably an immobilized Astigmatid prey, such as an immobilized Astigmatid prey, in particular a Carpoglyhus prey, having immobilized life stages comprising immobilized eggs, and

c. optionally a carrier, such as a carrier material selected from sawdust, wheat bran, buckwheat husks, rice husks or millet husks, or comprising a mixture thereof, preferably a carrier having carrier elements comprising mite shelters.

It is a further object of the present invention to disclose the biological control composition as defined in any of the above, wherein at least 10% of female individuals of the population is capable of reproduction on a non-tetranychid arthropod prey, preferably on an immobilized non-tetranychid arthropod prey, such as on a non-phytophagous prey, preferably on an Astigmatid prey, most preferably on an immobilized Astigmatid prey, such as an immobilized Astigmatid prey, in particular a Carpoglyhus prey, having immobilized life stages comprising immobilized eggs.

It is a further object of the present invention to disclose the biological control composition as defined in any of the above, wherein at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of female individuals of the population are capable of reproducing on a non-tetranychid arthropod prey, preferably on an immobilized non-tetranychid arthropod prey, such as on a non-phytophagous prey, preferably on an Astigmatid prey, most preferably on an immobilized

Astigmatid prey, such as an immobilized Astigmatid prey, in particular a Carpoglyhus prey, having immobilized life stages comprising immobilized eggs.

It is a further object of the present invention to disclose the biological control composition as defined in any of the above, wherein the at least 10% of female individuals of the population are capable of oviposition on immobilized Astigmatid prey having immobilized life stages comprising immobilized eggs.

It is a further object of the present invention to disclose the biological control composition as defined in any of the above, wherein the population has a daily oviposition rate of at least at least 0.50, such as ≥0.55, ≥0.60, ≥0.65, ≥0.70, ≥0.75, ≥0.80, ≥0.90, ≥0.95, ≥1.00, ≥1.05, ≥1.10, ≥1.15, ≥1.20, ≥1.25, ≥1.30, ≥1.35, ≥1.40, ≥1.45, ≥1.50, ≥1.55, ≥1.60, ≥1.65, ≥1.70, ≥1.75, ≥1.80, ≥1.85, ≥1.90, ≥1.95, or at least 2.00 eggs/day/female.

It is a further object of the present invention to disclose the biological control composition as defined in any of the above, wherein the population has a daily oviposition rate of at least at least 0.50, such as ≥0.55, ≥0.60, ≥0.65, ≥0.70, ≥0.75, ≥0.80, ≥0.90, ≥0.95, ≥1.00, ≥1.05, ≥1.10, ≥1.15, ≥1.20, ≥1.25, ≥1.30, ≥1.35, ≥1.40, ≥1.45, ≥1.50, ≥1.55, ≥1.60, ≥1.65, ≥1.70, ≥1.75, ≥1.80, ≥1.85, ≥1.90, ≥1.95, or at least 2.00 eggs/day/female, when using the non-tetranychid arthropod prey as the sole food source .

It is a further object of the present invention to disclose the biological control composition as defined in any of the above, wherein the at least 10% of female individuals are capable of completing a full ontogenetic cycle on the non-Tetranychid arthropod prey, when using the non-tetranychid arthropod prey as the sole food source.

It is a further object of the present invention to disclose the biological control composition as defined in any of the above, wherein the population is characterized by juvenile and/or female survival rate of at least 40%, preferably at least 45%, 50%, 55%, 60%, 65%, 70%, 75%, 80%, 85%, 90%, or at least 95% on the non-Tetranychid prey.

It is a further object of the present invention to disclose the biological control composition as defined in any of the above, wherein the at least 10% of female individuals of the population are characterized by capability to produce female offspring in a number of subsequent generations, wherein the number of subsequent generations is at least 1, such as at least 2, such as in at least 3, 4, 5, 6, 7, 8, 9 at least 10 generations.

It is a further object of the present invention to disclose the biological control composition as defined in any of the above, wherein the population is characterized by a daily reproduction rate in the range of about 1.10 -1.40, such as 1.15-1.40, 1.20-1.40, 1.25-1.40, 1.30-1.40, or 1.10-1.35, 1.10-1.30, 1.10-1.25, 1.10-1.20 on the non-Tetranychid prey.

It is a further object of the present invention to disclose the biological control composition as defined in any of the above, wherein female individuals have predatory behavior towards individuals of a Tetranychid species, preferably predatory behavior characterized by a daily reproduction rate of at least 10, preferably at least 15, more preferably at least 19 eggs per female per 5 days.

It is a further object of the present invention to disclose the biological control composition as defined in any of the above, wherein the population has an increased reproduction rate as compared to a control Phytoseiulus predatory population of the same species comprising a fraction of female individuals capable of reproduction on immobilized Astigmatid prey having immobilized life stages comprising immobilized eggs, lower than 10%.

It is a further object of the present invention to disclose a biological control composition comprising Phytoseiulus predatory individuals, wherein the population is characterized by a daily oviposition rate of at least 0.50, such as ≥0.55, ≥0.60, ≥0.65, ≥0.70, ≥0.75, ≥0.80, ≥0.90, ≥0.95, ≥1.00, ≥1.05, ≥1.10, ≥1.15, ≥1.20, ≥1.25, ≥1.30, ≥1.35, ≥1.40, ≥1.45, ≥1.50, ≥1.55, ≥1.60, ≥1.65, ≥1.70, ≥1.75, ≥1.80, ≥1.85, ≥1.90, ≥1.95, or at least 2.00 eggs/day/female while preying on non-tetranychid arthropod prey, preferably on an immobilized non-tetranychid arthropod prey, such as on a non-phytophagous prey, preferably on an Astigmatid prey, most preferably on an immobilized Astigmatid prey, such as an immobilized Astigmatid prey, in particular a Carpoglyphus prey, having immobilized life stages comprising immobilized eggs.

It is a further object of the present invention to disclose the biological control composition as defined in any of the above, wherein the population is characterized by a juvenile and/or female survival rate of at least 40%, preferably at least 45%, 50%, 55%, 60%, 65%, 70%, 75%, 80%, 85%, 90%, or at least 95% on the non-Tetranychid prey.

It is a further object of the present invention to disclose the biological control composition as defined in any of the above, wherein the at least 10% of female individuals of the population are characterized by capability to produce female offspring in a number of subsequent generations, wherein the number of subsequent generations is at least 1, such as at least 2, such as in at least 3, 4, 5, 6, 7, 8, 9 at least 10 generations.

It is a further object of the present invention to disclose the biological control composition as defined in any of the above, wherein the population is characterized by a daily reproduction rate in the range of about 1.10 -1.40, such as 1.15-1.40, 1.20-1.40, 1.25-1.40, 1.30-1.40, or 1.10-1.35, 1.10-1.30, 1.10-1.25, 1.10-1.20.

It is a further object of the present invention to disclose the biological control composition as defined in any of the above, wherein at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of female individuals of the population are capable of reproducing on a non-tetranychid arthropod prey, preferably on an immobilized non-tetranychid arthropod prey, such as on a non-phytophagous prey, preferably on an Astigmatid prey, most preferably on an immobilized Astigmatid prey, such as an immobilized Astigmatid prey, in particular a Carpoglyphus prey, having immobilized life stages comprising immobilized eggs.

It is a further object of the present invention to disclose biological control composition comprising Phytoseiulus predatory individuals, wherein the population is characterized by improved reproduction on non-tetranychid arthropod prey, preferably on an immobilized non-tetranychid arthropod prey, such as on a non-phytophagous prey, preferably on an Astigmatid prey, most preferably on an immobilized Astigmatid prey, such as an immobilized Astigmatid prey, in particular a Carpoglyphus prey, having immobilized life stages comprising immobilized eggs, as compared to a control Phytoseiulus predatory population of the same species comprising a fraction of female individuals capable of reproduction on immobilized Astigmatid prey having immobilized life stages comprising immobilized eggs, lower than 10%.

It is a further object of the present invention to disclose the biological control composition as defined in any of the above, wherein the improved reproduction on a non-tetranychid arthropod prey is characterized by at least one of: increased daily reproduction rate, increased daily oviposition rate, increased survival rate, increased percentage of female individuals reproducing on said prey, and improved predatory behavior towards a Tetranychidae.

It is a further object of the present invention to disclose the biological control composition as defined in any of the above, wherein predatory individuals are from a species selected from Phytoseiulus fragariae, Phytoseiulus longipes, Phytoseiulus macropilis, Phytoseiulus persimilis and Phytoseiulus robertsi.

It is a further object of the present invention to disclose the biological control composition as defined in any of the above, wherein the reproduction on a non-tertranychid prey is reproduction on an Astigmatid mite species selected from:

i) Carpoglyphidae such as from the genus Carpoglyphus e.g. Carpoglyphus lactis;

ii) Pyroglyphidae such as from the genus Dermatophagoides e.g. Dermatophagoides pteronysinus, Dermatophagoides farinae; from the genus Euroglyphus e.g. Euroglyphus longior, Euroglyphus maynei; from the genus Pyroglyphus e.g. Pyroglyphus africanus;

iii) Glycyphagidae such as from the subfamily Ctenoglyphinae, such as from the genus Diamesoglyphus e.g. Diamesoglyphus intermediusor from the genus Ctenoglyphus, e.g. Ctenoglyphus plumiger, Ctenoglyphus canestrinii, Ctenoglyphus palmifer; the subfamily Glycyphaginae, such as from the genus Blomia, e.g. Blomia freemani or from the genus Glycyphagus, e.g. Glycyphagus ornatus, Glycyphagus bicaudatus, Glycyphagus privatus, Glycyphagus domesticus, or from the genus Lepidoglyphus e.g. Lepidoglyphus michaeli, Lepidoglyphus fustifer, Lepidoglyphus destructor, or from the genus Austroglycyphagus, e.g. Austroglycyphagus geniculatus; from the subfamily Aeroglyphinae, such as from the genus Aeroglyphus, e.g. Aeroglyphus robustus; from the subfamily Labidophorinae, such as from the genus Gohieria, e.g. Gohieria fusca; or from the subfamily Nycteriglyphinae such as from the genus Coproglyphus, e.g. Coproglyphus stammeri or from the subfamily Chortoglyphidae, such as the genus Chortoglyphus e.g. Chortoglyphus arcuatus and more preferably is selected from the subfamily Glycyphaginae, more preferably is selected from the genus Glycyphagus or the genus Lepidoglyphus most preferably selected from Glycyphagus domesticus or Lepidoglyphus destructor;

iv) Acaridae such as from the genus Tyrophagus e.g. Tyrophagus putrescentiae, Tyrophagus tropicus, from the genus Acarus e.g. Acarus siro, Acarus farris, Acarus gracilis; from the genus Lardoglyphus e.g. Lardoglyphus konoi, from the genus Thyreophagus, such as Thyreophagus entomophagus; from the genus Aleuroglyphus, e.g. Aleuroglyphus ovatus;

v) Suidasiidae such as from the genus Suidasia, such as Suidasia nesbiti, Suidasia pontifica or Suidasia medanensis.

It is a further object of the present invention to disclose the Phytoseiulus persimilis predatory mite population as defined in any of the above, or the composition as defined in any of the above, or the biological control composition as defined in any of the above, wherein the immobilized Astigmatid prey is selected from the group consisting of immobilized mites, non-viable mites, non-hatching eggs, non-viable eggs and a combination thereof.

It is a further object of the present invention to disclose a rearing composition comprising: predatory mite population comprising at least one mite species of the genus Phytoseiulus, and a prey mite population comprising at least one species from the order Astigmata.

It is a further object of the present invention to provide a rearing composition comprising: a predatory mite population comprising at least one mite species of the genus Phytoseiulus, and a prey mite population comprising individuals of at least one mite species from the order Astigmata, wherein said predatory mite population is capable of oviposition for at least 2 generations, further wherein said Astigmata prey is selected from the group consisting of non-viable mites, non-viable eggs and a combination thereof.

It is a further object of the present invention to disclose the rearing composition as defined in any of the above, wherein said predatory mite is capable of oviposition for at least 10 generations reared on said Astigmata prey individuals.

It is a further object of the present invention to disclose the rearing composition as defined in any of the above, wherein said predatory mite population exhibits an increased reproduction rate trait as compared to a control predatory mite population lacking said trait.

It is a further object of the present invention to disclose the rearing composition as defined in any of the above, wherein said predatory mite population exhibits a daily reproduction rate in the range of about 1.15 -1.2.

It is a further object of the present invention to disclose the rearing composition as defined in any of the above, wherein said predatory mite population is characterized by a beige-white color.

It is a further object of the present invention to disclose the rearing composition as defined in any of the above, wherein said composition is absent of a fungus reducing agent.

It is a further object of the present invention to disclose the rearing composition as defined in any of the above, wherein said predatory mite species is selected from the group consisting of Phytoseiulus fragariae, Phytoseiulus longipes, Phytoseiulus macropilis, Phytoseiulus persimilis and Phytoseiulus robertsi.

It is a further object of the present invention to disclose the rearing composition as defined in any of the above, wherein said predatory mite species is Phytoseiulus persimilis.

It is a further object of the present invention to disclose the rearing composition as defined in any of the above, wherein the species from the order Astigmata is belonging to a family selected from the group consisting of Carpoglyphidae, Pyroglyphidae, Acaridae and Glycyphagidae.

It is a further object of the present invention to disclose the rearing composition as defined in any of the above, wherein the species from the order Astigmata comprises members from the family Carpoglyphidae, such as the genus Carpoglyphus, e.g. Carpoglyphus lactis, Carpoglyphus munroi; from the family Glycyphagidae such as the genus Glycyphagus, e.g. Glycyphagus domesticus, from the genus Lepidoglyphus, e.g. Lepidoglyphus destructor; from the family Pyroglyphidae such as the genus Dermatophagoides, e.g. Dermatophagoides farinae, Dermatophagoides pteronisinus, form the family Acaridae, such as the genus Tyrophagus, e.g. Tyrophagus putrescentiae.

It is a further object of the present invention to disclose the rearing composition as defined in any of the above, wherein said Astigmata prey population is in a frozen form. The term in a frozen form in the context of this invention should be understood as immobilized by freezing.

It is a further object of the present invention to disclose the rearing composition as defined in any of the above, wherein said Astigmata prey population comprises a mixture comprising non-viable frozen developmental stages of juvenile mites.

It is a further object of the present invention to disclose the rearing composition as defined in any of the above, wherein said composition comprises at least one mite species of the genus Phytoseiulus and a mixture comprising non-viable frozen developmental stages of C. lactis juvenile mites and sawdust or another carrier material.

It is a further object of the present invention to disclose the rearing composition as defined in any of the above, wherein said composition comprises P. persimilis and a mixture comprising non-viable frozen developmental stages of C. lactis juvenile mites and sawdust or another carrier material.

It is a further object of the present invention to disclose the rearing composition as defined in any of the above, wherein said Astigmata prey population comprises non-viable C. lactis eggs.

It is a further object of the present invention to disclose the rearing composition as defined in any of the above, wherein said Astigmata prey population comprises non-viable eggs and non-viable juvenile mites in a 1:1 ratio (w/w).

It is a further object of the present invention to disclose the rearing composition as defined in any of the above, wherein said composition further comprises a carrier such as sawdust, bran or another carrier material.

It is a further object of the present invention to disclose the rearing composition as defined in any of the above, wherein said predator population reared on said mite species from the order Astigmata, is reproduced by an average rate of at least about 15% per day, particularly by a range of 15% to 25% per day.

It is a further object of the present invention to disclose the rearing composition as defined in any of the above, wherein said Astigmatid individuals are treated by a treatment selected from the group consisting of: thermal treatment, such as freezing, heating, cold-shock or heat-shock treatment; chemical treatment, such as gas or fume treatment; radiation treatment, such as UV, microwave, gamma irradiation or X-ray treatment; mechanical treatment, such as vigorous shaking, or stirring, subjecting to shear forces, collision; gas pressure treatment, such as ultrasound treatment, pressure changes, pressure drops; electrical treatment, such as electrocution; immobilizing with an adhesive; immobilization by starvation, such as induced by water or food deprivation; immobilization by suffocation or anoxia treatment, such as by temporarily eliminating oxygen from the atmosphere or replacing oxygen by another gas and any combination thereof.

It is a further object of the present invention to disclose the rearing composition as defined in any of the above, wherein said composition comprises P. persimilis, and a mixture comprising non-viable C. lactis eggs and sawdust or another carrier material.

It is a further object of the present invention to disclose the rearing composition as defined in any of the above, wherein said composition comprises P. persimilis, and a mixture comprising non-viable C. lactis mites and sawdust or another carrier material.

It is a further object of the present invention to disclose the rearing composition as defined in any of the above, wherein said composition comprises a Phytoseiulus persimilis predatory mite population, and dead C. lactis individuals as a prey mite population, further wherein said Phytoseiulus persimilis predatory mite population has a daily reproduction rate in the range of about 1.15-1.2.

It is a further object of the present invention to disclose the rearing composition as defined in any of the above, wherein said composition comprises a Phytoseiulus persimilis predatory mite population and dead individuals of at least one species belonging to the Astigmata order selected from the group consisting of: Carpoglyphus lactis, Lepidoglyphus destructor, Glycifagus domestics, Dermatophagoides farinae and Dermatophagoides pteronisinus.

It is a further object of the present invention to disclose the rearing composition as defined in any of the above, wherein said prey mite population further comprises a mite species of the family Phytoseiidae.

It is a further object of the present invention to disclose the rearing composition as defined in any of the above, wherein said prey mite species of the family Phytoseiidae is non-viable.

It is a further object of the present invention to disclose the rearing composition as defined in any of the above, wherein said composition is capable of controlling a crop pest.

It is a further object of the present invention to disclose the rearing composition as defined in any of the above, wherein said crop pest is selected from the group of mite pests, particularly members of the Acari family Tetranychidae such as twospotted spider mite, more particularly spider mite species, especially Tetranychus, Panonychus and various other mite species.

It is a further object of the present invention to disclose the rearing composition as defined in any of the above, wherein said composition is capable of reducing said crop pest counts by at least 50%.

It is a further object of the present invention to disclose the rearing composition as defined in any of the above formulated for controlled release of said predatory mites on a crop plant.

It is a further object of the present invention to disclose the rearing composition as defined in any of the above contained in a container configured for controlled release of said predatory mites on a crop plant.

It is a further object of the present invention to disclose the rearing composition as defined in any of the above wherein said predatory mites are capable slowly and continuously released from said container to said crop during a period of about three weeks.

It is a further object of the present invention to disclose a method for rearing predatory mite population comprising at least one mite species of the genus Phytoseiulus, the method comprising: (a) providing a composition according to any one of claims 1 to 24; and (b) allowing individuals of the predatory mite population to prey on individuals of the Astigmatid population for at least 2 generations.

It is a further object of the present invention to disclose a method for rearing predatory mite population comprising at least one mite species of the genus Phytoseiulus, the method comprising: (a) providing a composition comprising a predatory mite population comprising at least one mite species of the genus Phytoseiulus, and a prey mite population comprising individuals of at least one mite species from the order Astigmata; (b) allowing individuals of the predatory mite population to prey on individuals of the Astigmatid population for at least 2 generations; wherein said Astigmata prey is selected from the group consisting of non-viable mites, non-viable eggs and a combination thereof.

It is a further object of the present invention to disclose the method as defined in any of the above, wherein the rearing population is maintained at a temperature range of 18-30° C., especially about 22° C.

It is a further object of the present invention to disclose the method as defined in any of the above, wherein the rearing population is maintained at a relative humidity of 70-90%, particularly about 85%.

It is a further object of the present invention to disclose the method as defined in any of the above, wherein said predatory mite is capable of oviposition for at least 2 generations, preferably for at least 10 generations, reared upon said Astigmata prey individuals.

It is a further object of the present invention to disclose the method as defined in any of the above, wherein said predatory mite population has a daily reproduction rate in the range of about 1.15 -1.2.

It is a further object of the present invention to disclose the method as defined in any of the above, wherein said predatory mite population is characterized by a beige-white color.

It is a further object of the present invention to disclose the method as defined in any of the above, wherein said composition is absent of a fungus reducing agent.

It is a further object of the present invention to disclose the method as defined in any of the above, wherein said predatory mite species is selected from the group consisting of Phytoseiulus fragariae, Phytoseiulus longipes, Phytoseiulus macropilis, Phytoseiulus persimilis and Phytoseiulus robertsi.

It is a further object of the present invention to disclose the method as defined in any of the above, wherein said predatory mite species is Phytoseiulus persimilis.

It is a further object of the present invention to disclose the method as defined in any of the above, wherein the species from the order Astigmata is belonging to a family selected from the group consisting of Carpoglyphidae, Pyroglyphidae, Acaridae and Glycyphagidae.

It is a further object of the present invention to disclose the method as defined in any of the above, wherein the species from the order Astigmata comprises members from the family Carpoglyphidae, such as the genus Carpoglyphus, e.g. Carpoglyphus lactis, Carpoglyphus munroi; from the family Glycyphagidae such as the genus Glycyphagus, e.g. Glycyphagus domesticus, from the genus Lepidoglyphus, e.g. Lepidoglyphus destructor; from the family Pyroglyphidae such as the genus Dermatophagoides, e.g. Dermatophagoides firinae, Dermatophagoides pteronisinus, form the family Acaridae, such as the genus Tyrophagus, e.g. Tyrophagus putrescentiae.

It is a further object of the present invention to disclose the method as defined in any of the above, wherein said Astigmata prey population is in a frozen form.

It is a further object of the present invention to disclose the method as defined in any of the above, wherein said Astigmata prey population comprises a mixture comprising non-viable frozen developmental stages of juvenile mites.

It is a further object of the present invention to disclose the method as defined in any of the above, wherein said composition comprises at least one mite species of the genus Phytoseiulus and a mixture comprising non-viable frozen developmental stages of C. lactis juvenile mites and sawdust or another carrier material.

It is a further object of the present invention to disclose the method as defined in any of the above, wherein said composition comprises P. persimilis and a mixture comprising non-viable frozen developmental stages of C. lactis juvenile mites and sawdust or another carrier material.

It is a further object of the present invention to disclose the method as defined in any of the above, wherein said Astigmata prey population comprises non-viable C. lactis eggs.

It is a further object of the present invention to disclose the method as defined in any of the above, wherein said Astigmata prey population comprises non-viable eggs and non-viable juvenile mites in a 1:1 ratio (w/w).

It is a further object of the present invention to disclose the method as defined in any of the above, wherein said composition further comprises a carrier such as sawdust, bran or another carrier material.

It is a further object of the present invention to disclose the method as defined in any of the above, wherein said predator population reared on said mite species from the order Astigmata, is reproduced by an average rate of at least about 15% per day, particularly by a range of 15% to 25% per day.

It is a further object of the present invention to disclose the method as defined in any of the above, wherein said Astigmatid individuals are treated by a treatment selected from the group consisting of: thermal treatment, such as freezing, heating, cold-shock or heat-shock treatment; chemical treatment, such as gas or fume treatment; radiation treatment, such as UV, microwave, gamma irradiation or X-ray treatment; mechanical treatment, such as vigorous shaking, or stirring, subjecting to shear forces, collision; gas pressure treatment, such as ultrasound treatment, pressure changes, pressure drops; electrical treatment, such as electrocution; immobilizing with an adhesive; immobilization by starvation, such as induced by water or food deprivation; immobilization by suffocation or anoxia treatment, such as by temporarily eliminating oxygen from the atmosphere or replacing oxygen by another gas and any combination thereof

It is a further object of the present invention to disclose the method as defined in any of the above, wherein said composition comprises P. persimilis, and a mixture comprising non-viable C. lactis eggs and sawdust or another carrier material.

It is a further object of the present invention to disclose the method as defined in any of the above, wherein said composition comprises P. persimilis, and a mixture comprising non-viable C. lactis mites and sawdust or another carrier material.

It is a further object of the present invention to disclose the method as defined in any of the above, wherein said composition comprises a Phytoseiulus persimilis predatory mite population, and dead C. lactis individuals as a prey mite population, further wherein said Phytoseiulus persimilis predatory mite population has a daily reproduction rate in the range of about 1.15 -1.2.

It is a further object of the present invention to disclose the method as defined in any of the above, wherein said composition comprises a Phytoseiulus persimilis predatory mite population and dead individuals of at least one species belonging to the Astigmata order selected from the group consisting of: Carpoglyphus lactis, Lepidoglyphus destructor, Glycifagus domestics, Dermatophagoides farinae and Dermatophagoides pteronisinus.

It is a further object of the present invention to disclose the method as defined in any of the above, wherein said prey mite population further comprises a mite species of the family Phytoseiidae.

It is a further object of the present invention to disclose the method as defined in any of the above, wherein said prey mite species of the family Phytoseiidae is non-viable.

It is a further object of the present invention to disclose a method for controlling a crop pest, the method comprising applying a composition as defined in any of the above to a field crop.

It is a further object of the present invention to disclose the method as defined in any of the above, wherein said crop pest is selected from the group of mite pests, particularly members of the Acari family Tetranychidae such as twospotted spider mite, more particularly spider mite species, especially Tetranychus, Panonychus and various other mite species.

It is a further object of the present invention to disclose the use of the composition as defined in any of the above for controlling a crop pest.

It is a further object of the present invention to disclose the use as defined in any of the above, wherein the crop pest is selected from a range of mite pests, particularly members of the Acari family Tetranychidae such as twospotted spider mite, more particularly spider mite species, especially Tetranychus, Panonychus and various other mite species.

It is a further object of the present invention to disclose the use as defined in any of the above, wherein the crop is selected from the group consisting of greenhouse grown crops, open field crops, vegetables, ornamentals, fruit trees, hops, cotton and strawberries.

It is a further object of the present invention to disclose a biological control agent (BCA) for controlling crop pests comprising a mixture of (a) at least one predatory mite species of the genus Phytoseiulus raised by the composition according to any one of claims 1 to 31, (b) optionally, prey mite individuals comprising at least one species from the order Astigmata, said Astigmata individuals are selected from the group consisting of non-viable mites, non-living eggs and a combination thereof; and (c) optionally a carrier material.

It is a further object of the present invention to disclose the BCA as defined in any of the above, wherein said predatory mite population is characterized by a beige-white color.

It is a further object of the present invention to disclose a container containing the composition according to any one of claims 1-29, said container configured to be hung on a crop plant, said container comprises an exit hole from which said predatory mites are slowly and continuously released to said crop during a period of about three weeks.

It is a further object of the present invention to disclose the container as defined in any of the above, wherein said container is selected from the group consisting of a sachet, a packet, a pouch, a pocket, a sack, a bottle and a bag.

It is a further object of the present invention to disclose the container as defined in any of the above, wherein said prey mites are in a frozen form.

It is a further object of the present invention to disclose the container as defined in any of the above, wherein said prey mites are frozen Astigmatid mite eggs.

It is a further object of the present invention to disclose the container as defined in any of the above, wherein said prey mites are frozen eggs of Carpoglyphus lactis.

It is a further object of the present invention to disclose the rearing composition as defined in any of the above, wherein at least partially of the Astigmata prey population is immobilized.

It is a further object of the present invention to disclose the rearing composition as defined in any of the above, wherein the Astigmata prey population is immobilized.

It is a further object of the present invention to disclose the rearing composition as defined in any of the above, wherein the Astigmata prey population comprises dead eggs and at least partially immobilized mites.

It is a further object of the present invention to disclose the rearing composition as defined in any of the above, wherein the Astigmata prey population comprises eggs and dead mites.

It is a further object of the present invention to disclose the rearing composition as defined in any of the above, wherein the Astigmata prey population comprises eggs and immobilized juvenile mites in a 1:1 ratio (w/w).

It is a further object of the present invention to disclose the rearing composition as defined in any of the above, wherein the mites are immobilized by an immobilization treatment selected from the group consisting of: thermal treatment, such as freezing, heating, cold-shock or heat-shock treatment; chemical treatment, such as gas or fume treatment; radiation treatment, such as UV, microwave, gamma irradiation or X-ray treatment; mechanical treatment, such as vigorous shaking, or stirring, subjecting to shear forces, collision; gas pressure treatment, such as ultrasound treatment, pressure changes, pressure drops; electrical treatment, such as electrocution; immobilizing with an adhesive; immobilization by starvation, such as induced by water or food deprivation; immobilization by suffocation or anoxia treatment, such as by temporarily eliminating oxygen from the atmosphere or replacing oxygen by another gas and any combination thereof.

It is a further object of the present invention to disclose the rearing composition as defined in any of the above, wherein the composition comprises P. persimilis, and a mixture comprising immobilized C. lactis and sawdust or another carrier material.

It is a further object of the present invention to disclose the rearing composition as defined in any of the above, wherein the immobilized C. lactis mites are dead mites.

It is a further object of the present invention to disclose the rearing composition as defined in any of the above, wherein the composition comprises a Phytoseiulus persimilis predatory mite population, and dead C. lactis individuals as a prey mite population, further wherein the Phytoseiulus persimilis predatory mite population is capable of oviposition for at least 2 generations, preferably for at least 10 generations.

It is a further object of the present invention to disclose the rearing composition as defined in any of the above, wherein the composition comprises a Phytoseiulus persimilis predatory mite population and dead individuals of at least one species belonging to the Astigmata order selected from the group consisting of: Carpoglyphus lactis, Lepidoglyphus destructor, Glycifagus domestics, Dermatophagoides farinae and Dermatophagoides pteronisinus.

It is a further object of the present invention to disclose the rearing composition as defined in any of the above, wherein the prey mite population further comprises a mite species of the family Phytoseiidae.

It is a further object of the present invention to disclose the rearing composition as defined in any of the above, wherein the Phytoseiidae prey mite species is of the genus Amblyseius, e.g. Amblyseius swirskii.

It is a further object of the present invention to disclose the rearing composition as defined in any of the above, wherein the prey mite species is Amblyseius swirskii.

It is a further object of the present invention to disclose the rearing composition as defined in any of the above, wherein the composition comprises a Phytoseiulus persimilis predatory mite population and prey mite population comprising Amblyseius swirskii mite species.

It is a further object of the present invention to disclose the rearing composition as defined in any of the above, wherein the Amblyseius swirskii mites are at least partially immobilized.

It is a further object of the present invention to disclose a rearing composition comprising: predatory mite population comprising at least one mite species of the genus Phytoseiulus, and a prey mite population comprising at least one species from the Phytoseiidae family.

It is a further object of the present invention to disclose the rearing composition as defined in any of the above, wherein the prey mite species is of the genus Amblyseius, e.g. Amblyseius swirskii.

It is a further object of the present invention to disclose the rearing composition as defined in any of the above, wherein the prey mite is immobilized.

It is a further object of the present invention to disclose the method as defined in any of the above, wherein at least partially of the Astigmata prey population is immobilized.

It is a further object of the present invention to disclose the method as defined in any of the above, wherein the Astigmata prey population is immobilized.

It is a further object of the present invention to disclose the method as defined in any of the above, wherein the Astigmata prey population comprises a mixture comprising dead frozen developmental stages of juvenile mites.

It is a further object of the present invention to disclose the method as defined in any of the above, wherein the composition comprises P. persimilis, and a mixture comprising dead frozen developmental stages of C. lactis juvenile mites and sawdust or another carrier material.

It is a further object of the present invention to disclose the method as defined in any of the above, wherein the Astigmata prey population comprises eggs and at least partially immobilized mites.

It is a further object of the present invention to disclose the method as defined in any of the above, wherein the Astigmata prey population comprises eggs and dead mites.

It is a further object of the present invention to disclose the method as defined in any of the above, wherein the Astigmata prey population comprises eggs and immobilized juvenile mites in a 1:1 ratio (w/w).

It is a further object of the present invention to disclose the method as defined in any of the above, wherein the mites are immobilized by an immobilization treatment selected from the group consisting of: thermal treatment, such as freezing, heating, cold-shock or heat-shock treatment; chemical treatment, such as gas or fume treatment; radiation treatment, such as UV, microwave, gamma irradiation or X-ray treatment; mechanical treatment, such as vigorous shaking, or stirring, subjecting to shear forces, collision; gas pressure treatment, such as ultrasound treatment, pressure changes, pressure drops; electrical treatment, such as electrocution; immobilizing with an adhesive; immobilization by starvation, such as induced by water or food deprivation; immobilization by suffocation or anoxia treatment, such as by temporarily eliminating oxygen from the atmosphere or replacing oxygen by another gas and any combination thereof.

It is a further object of the present invention to disclose the method as defined in any of the above, wherein the composition comprises P. persimilis, and a mixture comprising immobilized C. lactis and sawdust or another carrier material.

It is a further object of the present invention to disclose the method as defined in any of the above, wherein the immobilized C. lactis mites are dead mites.

It is a further object of the present invention to disclose the method as defined in any of the above, wherein the composition comprises a Phytoseiulus persimilis predatory mite population, and dead C. lactis individuals as a prey mite population, further wherein the Phytoseiulus persimilis predatory mite population is capable of oviposition for at least 2 generations, preferably for at least 10 generations.

It is a further object of the present invention to disclose the method as defined in any of the above, wherein the composition comprises a Phytoseiulus persimilis predatory mite population and dead individuals of at least one species belonging to the Astigmata order selected from the group consisting of: Carpoglyphus lactis, Lepidoglyphus destructor, Glycifagus domestics, Dermatophagoides farinae and Dermatophagoides pteronisinus.

It is a further object of the present invention to disclose the method as defined in any of the above, wherein the prey mite population further comprises a mite species of the family Phytoseiidae.

It is a further object of the present invention to disclose the method as defined in any of the above, wherein the prey mite species is of the genus Amblyseius, e.g. Amblyseius swirskii.

It is a further object of the present invention to disclose the method as defined in any of the above, wherein the prey mite species is Amblyseius swirskii.

It is a further object of the present invention to disclose the method as defined in any of the above, wherein the composition comprises a Phytoseiulus persimilis predatory mite population and prey mite population comprising Amblyseius swirskii mite species.

It is a further object of the present invention to disclose the method as defined in any of the above, wherein the Amblyseius swirskii mites are at least partially immobilized.

It is a further object of the present invention to disclose a method for rearing predatory mite population comprising at least one mite species of the genus Phytoseiulus, the method comprising: (a) providing a composition according to claim 26; and (b) allowing individuals of the predatory mite population to prey on individuals of the Phytoseiidae family population.

It is a further object of the present invention to disclose the method as defined in above, wherein the predatory mite species is selected from the group consisting of Phytoseiulus fragariae, Phytoseiulus longipes, Phytoseiulus macropilis, Phytoseiulus persimilis and Phytoseiulus robertsi.

It is a further object of the present invention to disclose the method as defined in any of the above, wherein the predatory mite species is Phytoseiulus persimilis.

It is a further object of the present invention to disclose the method as defined in any of the above, wherein the prey mite species is of the genus Amblyseius, e.g. Amblyseius swirskii.

It is a further object of the present invention to disclose the method as defined in any of the above, wherein the prey mite is immobilized.

It is a further object of the present invention to disclose a biological control product for controlling crop pests comprising a mixture of (a) Phytoseiulus persimilis predatory mite individuals raised by the composition as defined in any of the above, (b) prey mite individuals comprising at least one species from the order Astigmata, and (c) optionally a carrier material.

It is a further object of the present invention to disclose the biological control product as defined above, wherein the species from the order Astigmata comprises members from the family Carpoglyphidae, such as the genus Carpoglyphus, e.g. Carpoglyphus lactis, Carpoglyphus munroi; from the family Glycyphagidae such as the genus Glycyphagus, e.g. Glycyphagus domesticus, from the genus Lepidoglyphus, e.g. Lepidoglyphus destructor; from the family Pyroglyphidae such as the genus Dermatophagoides, e.g. Dermatophagoides firinae, Dermatophagoides pteronisinus, from the family Acaridae, such as the genus Tyrophagus, e.g. Tyrophagus putrescentiae.

It is a further object of the present invention to disclose a biological control product for controlling crop pests comprising a mixture of (a) Phytoseiulus persimilis predatory mite individuals raised by the composition as defined in any of the above, (b) prey mite individuals comprising at least one species from the Phytoseiidae family, and (c) optionally a carrier material.

It is a further object of the present invention to disclose a biological control product for controlling crop pests comprising predatory mite individuals of the genus Phytoseiulus raised by the composition as defined in any of the above.

It is a further object of the present invention to disclose the composition as defined in any of the above, formulated for controlled release of the predatory mites on a crop plant.

It is a further object of the present invention to disclose a container containing the composition as defined in any of the above, the container configured to be hung on a crop plant, the container comprises an exit hole from which the predatory mites are slowly and continuously released to the crop during a period of about three weeks.

It is a further object of the present invention to disclose the container as defined above, wherein the container is selected from the group consisting of a sachet, packet, pouch, pocket, sack and a bag.

It is a further object of the present invention to disclose the container as defined in any of the above, wherein the prey mites are frozen astigmatid mite eggs.

It is a further object of the present invention to disclose the container as defined in any of the above, wherein the prey mites are frozen eggs of Carpoglyphus lactis.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings; wherein:

FIG. 1 is a photographic illustration of different developmental stages of P. persimilis reared on dead or immobilized Carpoglyphus lactis (C. lactis) mites;

FIG. 2 is a photographic illustration of P. persimilis reared on dead or immobilized Carpoglyphus lactis (C. lactis) mites, as an embodiment of the present invention;

FIG. 3 is a graphic representation describing the daily multiplication rate of a P. persimilis population, feeding on a mixture of dead C. lactis eggs and dead mobile stages during a 14 weeks period;

FIG. 4 is a graphic representation of the percentage of P. persimilis showing feeding signs, as appeared by their body's shape and color;

FIG. 5 is a graphic representation of juvenile survival of P. persimilis reared on Astigmatid prey individuals of different families;

FIG. 6 graphically illustrates differences between daily reproduction rate of P. persimilis population sources (P+ and P−) reared upon C. lactis as a prey; the P+ population was bred and selected for improved adaptation for C. lactis as a prey; the P− population is the commercially available control P. persimilis population;

FIG. 7 is a graphic representation of the ability of P. persimilis predatory mites of the present invention to locate Tetranychid prey;

FIG. 8 is a graphic representation of the ability to control a spider mite population by treatment with P. persimilis population reared on non-Tetranychid prey, as compared to conventionally reared P. persimilis commercial population treatment, as measured by the number of predators and spider mites found in each sampling week under the different treatments (FIG. 8A) and by the spider mites control index found three weeks after predator's introduction at each treatment (FIG. 8B);

FIG. 9 graphically illustrates mites release rate as a function of number of days from experimental setup;

FIG. 10 graphically illustrates P. persimilis (Pp) and spider mite counts of plants exposed to the slow release system of the present invention, as compared to control plants;

FIG. 11 presents combinations of the percentage of females capable of reproducing on the non-Tetranychid arthropod prey (P) combined with values for the daily oviposition rate (O), as particularly envisaged for use in the different aspects of the present invention;

FIG. 12 presents combinations of the percentage of females capable of reproducing on the non-Tetranychid arthropod prey (P) combined with values for the percentage juvenile survival (J), as particularly envisaged for use in the different aspects of the present invention;

FIG. 13 presents combinations of the percentage of females capable of reproducing on the non-Tetranychid arthropod prey (P) combined with values for the percentage female survival (F), as particularly envisaged for use in the different aspects of the present invention;

FIG. 14 presents combinations of the percentage of females capable of reproducing on the non-Tetranychid arthropod prey (P) combined with values for the daily reproduction rate lambda (R), as particularly envisaged for use in the different aspects of the present invention;

FIG. 15 presents combinations of the Phytoseiulus species with (groups of) Astigmatid mites specifically envisaged for use in embodiments of the different aspects of the present invention;

FIG. 16 presents combinations of the combinations of the Phytoseiulus species x (groups of) Astigmatid mites (indicated by the PA1-PA270 reference numbers of FIG. 15) with the percentage of females capable of reproducing on the non-Tetranychid arthropod prey (P) x values for the percentage female survival (F) (indicated by the PF1-PF330 reference numbers FIG. 13); and

FIG. 17 presents further combinations of the combinations of the Phytoseiulus species x (groups of) Astigmatid mites (indicated by the PA1-PA270 reference numbers of FIG. 15) with the percentage of females capable of reproducing on the non-Tetranychid arthropod prey (P) x values for oviposition rate (indicated by the PO1-PO638 reference numbers of FIG. 13).

### DETAILED DESCRIPTION OF THE INVENTION

The twospotted spider mite, Tetranychus urticae Koch, is the major spider mite pest of ornamental plants and vegetable crops grown in greenhouses. Furthermore, this ubiquitous spider mite is a serious pest of numerous ornamental plants in home landscapes and is of considerable importance as a pest of food and fiber crops throughout the world (van de Vrie et al,. 1972). The predacious phytoseiid mite Phytoseiulus persimilis is the major species used to control twospotted spider mites in greenhouse as well as open field crops.

Phytoseiulus persimilis is a predatory mite which specializes on a diet of spider mites. Spider mites are vegetarian mites (phytophagous mites) and therefore require rearing on plants, which is undesirable since it involves complex operations and high rearing costs.

The present invention provides for the first time alternative method for rearing P. persimilis and other mite species of the genus Phytoseiulus. The current invention shows, against the conventional thinking, that mite species of the genus Phytoseiulus, e.g. P. persimilis, could broaden its dietary range, and could be reared on other preys, which are cheaper to produce and therefore much more desirable. The alternative prey mites are mostly Astigmatic mites that feed on stored products and are therefore significantly cheaper to produce.

According to one embodiment, the present invention provides a system and method for using mites (especially dead or otherwise immobilized mites) of the species Carpoglyphus lactis (Cl) or other Astigmatic mite as an alternative food for mite species of the Phytoseiulus genus, such as Phytoseiulus persimilis.

It is shown by the present invention that mite species of the genus Phytoseiulus, especially Phytoseiulus persimilis, can complete its life cycle and reproduce when feeding on dead mites belonging to the order Astigmata (within the Arachnida class).

The present invention is aimed at developing a system for the production of mite species of the genus Phytoseiulus, e.g. Phytoseiulus persimilis, on a diet comprising Astigmatic mites. The system is based on the following components:
1. The predator—specifically Phytoseiulus persimilis and more generally mites of the genus Phytoseiulus.
2. The prey—a mite species, possibly Carpoglyphus lactis, Glyciphagus domesticus, Lepidoglyphus destructor, Dermatophagoides farinae, Dermatophagoides pteronisinus or other Astigmatic mite, or other mite species such as Amblyseius swirskii.
3. The rearing system—the specific setup in which the mites are reared, comprising the rearing media, the way the prey mite is presented to the predator, the prey developmental stage and other factors.

The following rearing methods are within the scope of the present invention:
1. The predator is reared on a living mixture of prey mites.
2. The predator receives a mixture of immobilized prey mites by means of freezing or by other means such as irradiation.
3. A certain developmental stage of the prey mite is extracted from the prey mite's population, and then served alive or dead as food to the predator.

It is noted that in all of the above optional rearing methods, the prey mite could be either the above mentioned Astigmatic mites, or other species.

With respect to the final biological control product, the following is within the scope of the present invention:
1. A mixture which contains both the predator and the prey mites, or the predator and specific stages of the prey mites used for feeding the predator.
2. A further option is extracting only the predators, so that the final product contains only the predators.

According to one aspect, the present invention provides a predatory mite population comprising Phytoseiulus predatory individuals. In the population at least 10% of female individuals is capable of reproduction on a non-tetranychid arthropod prey. Within the present invention, at least 10% should be construed as meaning at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. In the present invention, at least 99% includes that substantially all of the female individuals is capable of reproduction on a non-tetranychid arthropod prey. At least 99% also includes that 100% of the female individuals is capable of reproduction on a non-tetranychid arthropod prey.

According to a further aspect, the present invention provides a predatory mite population comprising Phytoseiulus predatory individuals, wherein the population is characterized by a daily oviposition rate of at least 0.55, such as ≥0.60, ≥0.65, ≥0.70, ≥0.75, ≥0.80, ≥0.90, ≥0.95, ≥1.00, ≥1.05, ≥1.10, ≥1.15, ≥1.20, ≥1.25, ≥1.30, ≥1.35, ≥1.40, ≥1.45, ≥1.50, ≥1.55, ≥1.60, ≥1.65, ≥1.70, ≥1.75, ≥1.80, ≥1.85, ≥1.90, ≥1.95, or ≥2.00 eggs/day/female while preying on non-tetranychid arthropod prey. This aspect of the invention includes embodiments wherein the percentage of female individuals capable of reproduction on a non-tetranychid arthropod prey is not specified (is unspecified).

According to yet a further aspect, the invention relates to a biological control composition wherein the composition comprises:
a. a predatory mite population comprising individuals of at least one mite species of the genus Phytoseiulus capable of reproduction on a non-tetranychid arthropod prey, preferably on an immobilized non-tetranychid arthropod prey, such as on a non-phytophagous prey, preferably on an Astigmatid prey, most preferably on an immobilized Astigmatid prey, such as an immobilized Astigmatid prey, in particular a Carpoglyhus prey, having immobilized life stages comprising immobilized eggs; and
b. a prey mite population comprising individuals of a non-tetranychid arthropod prey, preferably an immobilized non-tetranychid arthropod prey, such as a non-phytophagous prey, preferably an Astigmatid prey, most preferably an immobilized Astigmatid prey, such as an immobilized Astigmatid prey, in particular a Carpoglyhus prey, having immobilized life stages comprising immobilized eggs, and
c. optionally a carrier, such as a carrier material selected from sawdust, wheat bran, buckwheat husks, rice husks or millet husks, or comprising a mixture thereof, preferably a carrier having carrier elements comprising mite shelters. A non-Tetranychid arthropod prey according to the present invention is a prey selected from arthropods other then Tetranychids. The non-Tetranychid arthropod prey may be a non-phytophagous prey, preferably an Astigmatid prey. In the present invention most preferably, an immobilized Astigmatid prey is used as the non-tetranychid arthropod prey, in particular an immobilized Astigmatid prey having immobilized life stages comprising immobilized eggs.

A further aspect of the invention relates to a biological control composition comprising Phytoseiulus predatory individuals, wherein the population is characterized by a daily oviposition rate of at least 0.50, such as ≥0.55, ≥0.60, ≥0.65, ≥0.70, ≥0.75, ≥0.80, ≥0.90, ≥0.95, ≥1.00, ≥1.05, ≥1.10, ≥1.15, ≥1.20, ≥1.25, ≥1.30, ≥1.35, ≥1.40, ≥1.45, ≥1.50, ≥1.55, ≥1.60, ≥1.65, ≥1.70, ≥1.75, ≥1.80, ≥1.85, ≥1.90, ≥1.95, or at least 2.00 eggs/day/female while preying on non-tetranychid arthropod prey, preferably on an immobilized non-tetranychid arthropod prey, such as on a non-phytophagous prey, preferably on an Astigmatid prey, most preferably on an immobilized Astigmatid prey, such as an immobilized Astigmatid prey, in particular a Carpoglyphus prey, having immobilized life stages comprising immobilized eggs.

According to a further aspect, the invention relates to a biological control composition comprising Phytoseiulus predatory individuals, wherein the population is characterized by improved reproduction on non-tetranychid arthropod prey, preferably on an immobilized non-tetranychid arthropod prey, such as on a non-phytophagous prey, preferably on an Astigmatid prey, most preferably on an immobilized Astigmatid prey, such as an immobilized Astigmatid prey, in particular a Carpoglyphus prey, having immobilized life stages comprising immobilized eggs, as compared to a control Phytoseiulus predatory population of the same species which was not exposed to a non-tetranychid arthropod prey and/or comprising a fraction of female individuals capable of reproduction on immobilized Astigmatid prey having immobilized life stages comprising immobilized eggs, lower than 10%.

According to certain embodiments of the different aspects of the invention, female individuals capable of reproduction on the non-tetranychid arthropod prey are female individuals capable of oviposition on non-Tetranychid arthropod prey, preferably on immobilized Astigmatid prey having immobilized life stages comprising immobilized eggs. As the skilled person will understand, oviposition capability relates to the capability to lay or produce eggs. Determining oviposition rates is within the ambit of the skill of the skilled person. The oviposition capability of the females preferably is determined after being fed for at least 4 days on the non-tetranychid prey, such as after 5 days or after 6 days.

The daily oviposition rate of a predatory mite population according to various aspects of the invention may be at least 0.50, such as ≥0.55, ≥0.60, ≥0.65, ≥0.70, ≥0.75, ≥0.80, ≥0.90, ≥0.95, ≥1.00, ≥1.05, ≥1.10, ≥1.15, ≥1.20, ≥1.25, ≥1.30, ≥1.35, ≥1.40, ≥1.45, ≥1.50, ≥1.55, ≥1.60, ≥1.65, ≥1.70, ≥1.75, ≥1.80, ≥1.85, ≥1.90, ≥1.95, or at least 2.00 eggs/day/female.

In some embodiments, the daily oviposition rate is at least 1 egg per day per female, particularly at least 1.4 eggs/day/female, more particularly between 1.4 -2 eggs/day/female. In main aspects of the present invention, a daily oviposition rate of at least 1 egg per day per female, particularly at least 1.4 eggs/day/female, more particularly between 1.4-2 eggs/day/female, is achieved when using the non-tetranychid arthropod prey as the sole food source for the Phytoseiulus predatory individuals. According to further aspects of the present invention, a daily oviposition rate of at least 1 egg per day per female, particularly at least 1.4 eggs/day/female, more particularly between 1.4 -2 eggs/day/female, is achieved when using the non-tetranychid arthropod prey as a food source for the Phytoseiulus predatory individuals in alternation with spider mites diet.

With in the present invention the term “at least” in the context of numerical values is considered equivalent with the meaning of the mathematical sign “≥”. The skilled person will understand that, being an average value for (the female part of) the poplation, the oviposition rate or egg production rate may have a fractional value not corresponding to whole eggs. The skilled person will also understand that a mite population having a daily oviposition rate of at least 0.50 eggs/day/female is capable to produce 0.5 eggs/day/female or more. Thus defined differently, a predatory mite population having a daily oviposition rate of ≥0.55, ≥0.60, ≥0.65, ≥0.70, ≥0.75, ≥0.80, ≥0.90, ≥0.95, ≥1.00, ≥1.05, ≥1.10, ≥1.15, ≥1.20, ≥1.25, ≥1.30, ≥1.35, ≥1.40, ≥1.45, ≥1.50, ≥1.55, ≥1.60, ≥1.65, ≥1.70, ≥1.75, ≥1.80, ≥1.85, ≥1.90, ≥1.95, or ≥2.00 eggs/day/female is capable of producing respectively 0.55, 0.60, 0.65, 0.70, 0.75, 0.80, 0.90, 0.95, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00 eggs/day/female. Again, if more eggs then the indicated numbers are produced, the indicated number of eggs are produced.

The daily oviposition rate of a predatory mite population according to other embodiments of the different aspects of the invention may be at least 0.50, such as ≥0.55, ≥0.60, ≥0.65, ≥0.70, ≥0.75, ≥0.80, ≥0.90, ≥0.95, ≥1.00, ≥1.05, ≥1.10, ≥1.15, ≥1.20, ≥1.25, ≥1.30, ≥1.35, ≥1.40, ≥1.45, ≥1.50, ≥1.55, ≥1.60, ≥1.65, ≥1.70, ≥1.75, ≥1.80, ≥1.85, ≥1.90, ≥1.95, or at least 2.00 eggs/day/female, when using the non-tetranychid arthropod prey as the sole food source or alternatively when preying on the non-tetranychid arthropod prey. Also in this case, defined differently, a predatory mite population having a daily oviposition rate of ≥0.55, ≥0.60, ≥0.65, ≥0.70, ≥0.75, ≥0.80, ≥0.90, ≥0.95, ≥1.00, ≥1.05, ≥1.10, ≥1.15, ≥1.20, ≥1.25, ≥1.30, ≥1.35, ≥1.40, ≥1.45, ≥1.50, ≥1.55, ≥1.60, ≥1.65, ≥1.70, ≥1.75, ≥1.80, ≥1.85, ≥1.90, ≥1.95, or ≥2.00 eggs/day/female is capable of producing respectively 0.55, 0.60, 0.65, 0.70, 0.75, 0.80, 0.90, 0.95, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00 eggs/day/female when using the non-tetranychid arthropod prey as the sole food source. When using the non-Tetranychid prey as the sole food source no other food is presented to the individuals of the predatory mite population. As stated before, if more eggs then the indicated numbers are produced, the indicated number of eggs are produced.

Capability to reproduce on non-Tetranychid arthropod prey according to certain embodiments of different aspects of the invention most preferably includes the capability of completing a full ontogenetic cycle, when using the non-tetranychid arthropod prey as the sole food source. Completion of the ontogenic cycle, as the skilled person will understand is the capability of individuals to develop from the earlies life stage to a subsequent earlies life stage in a second generation, viz. for predatory mites development from a (parent) egg to an (offspring) egg in a next generation or defined differently development from an egg to a sexually mature female individual producing a number of eggs. The skilled person will know and understand that for many predatory mite species, including Phytoseiulus species, copulation with a male individual is required for egg production in females. If a population is capable to complete the ontogentic cycle on a certain food source, it can in theory perpetually cycle through multiple generations on that food source.

Capability to reproduce on non-Tetranychid arthropod prey according to certain embodiments of different aspects of the invention is characterized by capability of female individuals to produce female offspring in a number of subsequent generations. The number of subsequent generations is at least 1, such as at least 2, such as in at least 3, 4, 5, 6, 7, 8, 9 at least 10 generations. The skilled person will understand that in case the number of subsequent generations, is at least 2, a full ontogenetic cycle is completed, as the female offspring of the female has produced (female) off spring. Thus the number of subsequent generations preferably is at least 2, such that at least one ontogentic cycle is completed.

Capability to reproduce on non-Tetranychid arthropod prey according to certain embodiments of different aspects of the invention may also include a juvenile and/or female survival rate of at least 40%, on the non-Tetranychid prey used as the sole food source. As the skilled person will understand, the juvenile survival rate is the percentage of juvenile life stages that is capable of developing to the adult stage.

Juvenile survival rates in the context of the present invention are determined as the percentage post embryonic (post-egg) stages that reach adulthood. The juvenile surfival rate is determined on the non-Tetranychid prey, preferably an immobilized Astigmatid prey having immobilized life stages comprising immobilized eggs, used as the sole food source. Juvenile survival is determined over a period of between 3 to 7 days, such as over a period of 2, 3, 4, 5, 6, or 7 days, most preferably during a period of 3 days. The female survival rate is the rate of mature females that survive on the non-Tetranychid prey, preferably an immobilized Astigmatid prey having immobilized life stages comprising immobilized eggs, when used as the sole food source. Female survival is determined over a period of 7 days. At least 40% for the juvenile surfival rate may be between 40% and 95%, such as 45%-90%, 50%-90%, 55-90%, 60%-90%, 65%-90%, 70%-90%, 75%-90%, 45%-85%, 50%-85%, 55-85%, 60%-85%, 65%-85%, 70%-85%, 75%-85%. At least 40% for the female survival rate may be at least 45%, at least 50%, at least 55%, at least 60%, at least 65%, at least 70%, at least 75%, at least 80%, at least 85%, at least 90%, at least 95%, at least 99%. At least 40% and all higher percentages mentioned, include substantially all and 100%.

In some embodiments, the juvenile and/or female survival rate is at least 60%, particularly at least 80% and up to 100%. In main aspects of the present invention, a juvenile and/or female survival rate of at least 60%, particularly at least 80% and up to 100%, is achieved when using the non-tetranychid arthropod prey as the sole food source for the Phytoseiulus predatory individuals. According to further aspects of the present invention, a juvenile and/or female survival rate of at least 60%, particularly at least 80% and up to 100%, is achieved when using the non-tetranychid arthropod prey as a food source for the Phytoseiulus predatory individuals in alternation with spider mites diet.

Capability to reproduce on non-Tetranychid arthropod prey according to certain embodiments of different aspects of the invention may also be characterized by a daily multiplication (or reproduction) rate λ in the range of about 1.10 -1.40, such as 1.15-1.40, 1.20-1.40, 1.25-1.40, 1.30-1.40, or 1.10-1.35, 1.10-1.30, 1.10-1.25, 1.10-1.20. The skilled person will understand that for values for λ above 1.0 the population increases, thus there is reproduction. The skilled person will further understand that due to starvation in a population, also under the circumstance that values for λ for a given population are somewhat below 1.0, individuals in the population may be reproducing (to a level that is not compensating for the level of starvation). The daily multiplication rate according to preferred embodiments relates to daily multiplication rates when using the non-Tetranychid arthropod prey as the sole food source. Although daily multiplication (or reproduction) rates above 1.0 have been observed by the inventors of the present invention for existing Phytoseiulus populations, these existing Phytoseiulus populations do not have daily multiplication (or reproduction) rates of 1.10 or above.

In some embodiments, the daily multiplication (or reproduction) rate λ is at least 1.15, particularly at least 1.2, more particularly 1.2-1.4. In main aspects of the present invention, a daily multiplication (or reproduction) rate λ of at least 1.15, particularly at least 1.2, more particularly 1.2-1.4, is achieved when using the non-tetranychid arthropod prey as the sole food source for the Phytoseiulus predatory individuals. According to further aspects of the present invention, a daily multiplication (or reproduction) rate λ of at least 1.15, particularly at least 1.2, more particularly 1.2-1.4, is achieved when using the non-tetranychid arthropod prey as a food source for the Phytoseiulus predatory individuals in alternation with spider mites diet.

In general, within the context of the present invention life stages parameters, such as oviposition rates and survival rates, completion of the ontogentic cycle and population growth rates of predatory mites may be determined at 22 degrees Celsius and 85% relative humidity, while food (the non-tetranychid arthropod prey) is not limiting (presented ad libidum).

According to preferred embodiments of the predatory mite population of the invention, predatory individuals have predatory behavior towards individuals of a Tetranychid species. Preferably female individuals have predatory behavior towards

Tetranychid individuals. If in the population at least 10% of the female individuals is capable of reproduction a non-Tetranychid arthropod prey, most preferably this at least 10% of the female individuals has predatory behavior towards individuals of a Tetranychid species. By maintaining predatory behavior towards individuals of a Tetranychid species, the predatory mite individuals can be used as biocontrol agents against the tetranychid species on which they predate. According to preferred embodiments, the predatory behavior towards individuals of a Tetranychid species may be a daily oviposition rate of at least 10, preferably at least 15, more preferably at least 19 eggs per female per 5 days.

According to some embodiments of the present invention, the above described predatory behavior towards individuals of a Tetranychid species is achieved when using the non-tetranychid arthropod prey as the sole food source for the reared Phytoseiulus predatory individuals. According to further aspects of the present invention the above described predatory behavior towards individuals of a Tetranychid species, is achieved when using the non-tetranychid arthropod prey as a food source for the reared Phytoseiulus predatory individuals in alternation with spider mites diet.

According to one embodiment of the different aspects of the invention, the invention provides a rearing composition comprising: a predatory mite population comprising at least one mite species of the genus Phytoseiulus, and a prey mite population comprising individuals of at least one mite species from the order Astigmata, wherein said predatory mite population is capable of oviposition for at least 2 generations, further wherein said Astigmata prey is selected from the group consisting of immobilized mites, preferably non-viable mites, non-hatching (immobilized) eggs, preferably non-viable eggs and a combination thereof.

It is within the scope of the present invention that the predatory mite is capable of oviposition for at least 10 generations and preferably more, having the Astigmata individuals as a prey.

It is further within the scope that the predatory mite population exhibits an increased reproduction rate trait, in particular when using Astigmatid mites as food source, as compared to a control predatory mite population, of the same species, lacking the aforementioned trait.

It is further within the scope that the predatory mite population of the present invention exhibits a daily reproduction rate in the range of about 1.15-1.2, in particular when using Astigmatid mites as food source.

It is further within the scope of the present invention that the predatory mite population is characterized by a beige-white color, when said Phytoseiulus predatory mite is reared upon said Astigmata prey as a food source.

It is within the scope of the present invention that the predators would have a different appearance than that of the common product containing P. persimilis mites reared on spider mites (white mites in the case of the present invention instead of the usual orange).

According to a further embodiment, the present invention shows for the first time that a population of P. persimilis successfully developed and reproduced on dead Carpoglyphus lactis for at least six months (about 25 generations).

It is emphasized that P. persimilis is herein surprisingly reported to complete its life cycle and reproduce on either non-phytophagous prey (prey that doesn't require to feed on living plants), or prey that doesn't consume phytophagous mites.

The present invention provides a mite composition which contains a Phytoseiulus persimilis rearing mite population, and a factitious host mite population comprising at least one species from the order Astigmata or from the family Phytoseiidae. Up until now, mite species of the genus Phytoseiulus, such as the important predator mite Phytoseiulus persimilis, were reared on their natural phytophagous mite diet which involves high costs and resources (such as providing appropriate plants in sufficient abundance, under greenhouse conditions).

The present invention solves the serious problem of rearing the main spider-mite controlling predator, Phytoseiulus persimilis, by rearing it in a cost effective and efficient way on a non-phytophagous alternative diet.

Accordingly, the invention provides a mite composition comprising: a rearing population of mite species of the genus Phytoseiulus, for example Phytoseiulus persimilis predatory mite species, a population of at least one species from the order Astigmata or from the family Phytoseiidae, and optionally a carrier.

According to one embodiment of the different aspects, the present invention provides a rearing composition comprising: predatory mite population comprising at least one mite species of the genus Phytoseiulus, and a prey mite population comprising at least one species from the order Astigmata.

According to a further embodiment of the different aspects, the present invention provides a method for rearing predatory mite population comprising at least one mite species of the genus Phytoseiulus, the method comprising: (a) providing a composition comprising a predatory mite population comprising at least one mite species of the genus Phytoseiulus, and a prey mite population comprising at least one species from the order Astigmata; and (b) allowing individuals of the predatory mite population to prey on individuals of the Astigmatid population.

According to a further embodiment of the different aspects, the present invention provides a rearing composition comprising: predatory mite population comprising at least one mite species of the genus Phytoseiulus, and a prey mite population comprising at least one species from the Phytoseiidae family.

According to a further embodiment of the different aspects, the present invention provides a method for rearing predatory mite population comprising at least one mite species of the genus Phytoseiulus, the method comprising: (a) providing a composition comprising: predatory mite population comprising at least one mite species of the genus Phytoseiulus, and a prey mite population comprising at least one species from the Phytoseiidae family; and (b) allowing individuals of the predatory mite population to prey on individuals of the Phytoseiidae family population.

In some embodiments of the different aspects, the prey population i.e. species from the order Astigmata or species from the Phytoseiidae family, is immobilized and/or not alive.

It is further within the scope of the different aspects that the Phytoseiulus persimilis predatory mite is capable of reproducing for at least 2 generations, preferably at least 10 generations, more preferably for at least 15 generations or more generations, feeding on the aforementioned Astigmata or Phytoseiid population, in particular an immobilized population.

The composition of the present invention provides a considerable number of advantages over previous combinations. In one aspect, the food material used to feed the prey during predator production will no longer be plants or phytophagous mites, but mites that live upon stored products, therefore providing a substantial cost saving.

In another aspect, the present invention provides a rearing composition comprising: predatory mite population comprising at least one mite species of the genus Phytoseiulus, and a prey mite population comprising at least one species from the Phytoseiidae family.

According to some further embodiments of the different aspects of the present invention, the predatory mite species is selected from the group consisting of Phytoseiulus fragariae, Phytoseiulus longipes, Phytoseiulus macropilis, Phytoseiulus persimilis and Phytoseiulus robertsi.

According to further embodiments of the present invention, the predatory mite species is Phytoseiulus persimilis.

According to yet further embodiments of the different aspects of the present invention, the prey mite species is of the genus Amblyseius, e.g. Amblyseius swirskii.

According to further embodiments of the different aspects of the present invention, the rearing composition comprises immobilized prey mites.

According to further aspects of the present invention, the prey mites are immobilized or dead mites.

According to further aspects, the present invention provides a method for controlling a crop pest, the method comprising applying a composition as defined in any of the above to a field crop.

According to further aspects, the present invention provides use of the composition as defined in any of the above for controlling a crop pest.

According to further aspects, the present invention provides a biological control product for controlling crop pests comprising a mixture of (a) Phytoseiulus persimilis predatory mite individuals raised by the composition as defined in any of the above, (b) prey mite individuals comprising at least one species from the order Astigmata, and (c) optionally a carrier material.

According to further aspects, the present invention provides a biological control product for controlling crop pests comprising a mixture of (a) Phytoseiulus persimilis predatory mite individuals raised by the composition according to any one of claims 26 to 31, and (b) prey mite individuals comprising at least one species from the Phytoseiidae family, and (c) optionally a carrier material.

The present invention further provides a slow release system (e.g. sachet) for mites, especially for mite species of the genus Phytoseiulus, particularly Phytoseiulus persimilis (P. persimilis) configured to be applied on a crop.

A core aspect of the innovative solution is that the predatory mites can reproduce within the system for several generations, while a certain proportion of the predatory mites continuously leaves the system and reaches the crop to control pests. This provides a continuous supply of mites to the crop without the need to apply them repeatedly by the farmer.

Embodiments of the slow release system provided by the present invention are based upon the following features:
1. Predatory mite individuals—P. persimilis or other mite species of the Phytoseiulus genus.
2. Food source for the predatory mites—a factitious prey or host for example, frozen eggs of Carpoglyphus lactis (C. lactis) or another astigmatic mite.
3. The predatory mites are combined with their factitious host at the same physical location. This is done by the following alternative approaches:
   a. Providing the predatory mites with their factitious host in a container such as a sachet, packet, pouch, pocket, sack or a bag configured to be hung on the crop plant, from which the mites would slowly and continuously be released to the crop during a period of about three weeks.
   b. Applying a mixture containing the predatory mites, a carrier and the factitious host as a food source, directly on the crop leaves. From this mixture, the predatory mites would slowly be released to the crop during a period of about three weeks.

It is noted that such slow release systems for predatory mites are highly desirable for P. persimilis since up until now, P. persimilis was known as a specialist (natural enemy) of spider mites and therefore reared upon spider mites diet. However, spider mites are not suitable to be used in this kind of mite release systems for crop protection for the following reasons:
Spider mites are pests themselves, and if applied alive, they may damage the crop.
Spider mites cannot reproduce without being supplied with plant material, therefore can't reproduce in a sachet.
Without being supplied with a food source, living spider mites die rapidly and shrivel (e.g. within few days).
If served dead, spider mites quickly shrivel and loose their nutritional value.
Spider mites are expensive to produce.

The present invention provides an unexpected technological solution for the above problem, which was not shown to be successful up until now. The solution is based upon using non-hatching (immobilized), in particular frozen, eggs of C. lactis or other astigmatid mite species as a factitious host for P. persimilis. Contrary to spider mites, non-hatching eggs (e.g. due to immobilization by freezing) of Astigmatids, in particular C. lactis, maintain their nutritional value for about three weeks. This innovative solution enables the prolonged release of P. persimilis predatory mites from a container or a mixture combining the predatory mite with its factitious host, applied directly on the crop plant for controlling pests.

As used herein the term “about” denotes ±10% of the defined amount or measure or value.

The term “controlled release” refers hereinafter to slow release, sustained-release, rapid release, designed to release in a prolonged controlled mode or fashion. In the context of the present invention, it refers to predatory mite release to the crop plant gradually over a specified period of time, e.g. throughout the day or over a week.

The term “slow release system” or a “device” or a “container” refers herein after to a sachet- type release system, e.g. a sachet, packet, pouch, pocket, sack, a bottle or a bag or any other device or means for releasing the composition or formulation of the present invention. In the context of the present invention, such a composition may comprise (i) Phytoseiulus predatory mites, (ii) Phytoseiulus predatory mites with a factitious host (dead astigmatid mite life-stages or other non-Tetranychid arthropod prey) (iii) a non-tetranychid arthropod prey, preferably an immobilized non-tetranychid arthropod prey, such as on a non-phytophagous prey, preferably on an Astigmatid prey, most preferably on an immobilized Astigmatid prey, such as an immobilized Astigmatid prey, in particular a Carpoglyhus prey, having immobilized life stages comprising immobilized eggs, and (iv) optionally a carrier. It is further included within the scope of the present invention that such a system or container refers to an apparatus, a unit, a device, a compartment, a member, strip or housing for slow release of beneficial insects or predatory mites available or known in the art, which gradually releases the beneficial insects or predatory mites. Having knowledge of such systems, the skilled sperson will understand that such a gradual release is opposed to immediate release.

It is also within the scope of the present invention that the Phytoseiulus predatory mite releasing system may be of any suitable type. In general the mite releasing system may comprise a container suitable for holding the individuals of the Phytoseiulus predatory mite (e.g. P. persimilis) and individuals of the factitious host mite (e.g. dead C. lactis eggs). The container comprises an opening and/or means for generating an exit opening for mobile stages of the Phytoseiulus predatory mite. Releasing systems of this type are known to the skilled person and various products are commercially available on the market, e.g. sachet-type releasing systems and other suitable types of releasing systems which are included within the scope of the present invention.

According to some aspects of the invention, a use of a non-Tetranychid arthropod species comprises applying individuals of a non-Tetranychid arthropod species on a target crop, preferably an immobilized non-Tetranychid arthropod species, such as a non-phytophagous prey, preferably an Astigmatid species, most preferably an immobilized Astigmatid species, such as an immobilized Astigmatid species, in particular a Carpoglyhus species, having immobilized life stages comprising immobilized eggs (for example a mixture of dead life stages, including dead eggs).

In a further embodiment, a mixture of eggs and mobile stages is applied to a crop plant to be infested with Phytoseiulus predatory mites. The purpose of applying the prey directly on the plant is to support a population of P. persimilis or other natural enemy to be established on the plant when tetranychid prey (the natural host of Phytoseiulus predatory species) is scarce. According to specific embodiments, devices for releasing the mobile stages of the prey, as disclosed in the application, are used.

The term “rearing composition” as used herein generally refers to a composition suitable for breeding, bringing up, raising, upbringing or propagating a mite species by sexual reproduction. A rearing composition comprises a rearing population of the mite species, in particular the Phytoseiulus species. A rearing population may comprise sexually mature adults from both sexes, and/or individuals of both sexes of other life stages, e.g. eggs, larvae and/or nymphs, which can mature to sexually mature adults. Alternatively the rearing population may comprise one or more fertilized females. In essence a rearing population is capable of increasing the number of its individuals by means of sexual reproduction. More specifically, the term “rearing composition” refers to a composition suitable for the commercial rearing of mites. It is herein acknowledged that mass rearing systems for predatory mites heavily depend on the availability of suitable prey for the predators. Therefore, there is a continuing need to improve rearing systems of both predatory mites and mites suitable as rearing prey. To solve this problem, the present invention provides a composition or system specifically adapted for effectively and efficiently rearing mite species of the genus Phytoseiulus, especially Phytoseiulus persimilis, a highly important predatory mite used for crop pest (spider mites) biological control. For the first time, Phytoseiulus persimilis is shown to complete its life cycle and reproduce, i.e. for at least 2 generations, by being reared on Astigmatid mite species or on Phytoseiidae prey mite species, e.g. Amblyseius swirskii.

The term “carrier” refers hereinafter to an inactive or inert substance or particles or vehicle. In a preferred embodiment the rearing composition of the present invention comprises a carrier for the individuals of the mite species. The carrier can be any solid material which is suitable to provide a carrier surface to the mite individuals. Examples of suitable carriers are plant materials such as bran (e.g. wheat), sawdust (e.g. fine sawdust), corn cob grits, vermiculite, Poaceae husks, such as millet husks, or rice husks, etc. According to further aspects of the invention, a carrier material may include sawdust, wheat bran, buckwheat husks, rice husks or millet husks, or comprising a mixture thereof, preferably a carrier having carrier elements comprising mite shelters.

The term “Phytoseiulus ” as used hereinafter refers to a genus of mites in the

Phytoseiidae family. This genus of predatory mites is most frequently used to control two-spotted spider mites in greenhouses and outdoor crops. It is within the scope of the present invention that the genus Phytoseiulus contains the following species: Phytoseiulus fragariae, Phytoseiulus longipes, Phytoseiulus macropilis, Phytoseiulus persimilis, Phytoseiulus robertsi and Mesoseiulus longipes (e.g. see https://www.benemite.com/mlongipes.htm). The Phytoseiulus predetoy mites are known as specialists for spider mites (mites of the family Tetranychidae) which are phytophagous mites.

The term “Phytoseiulus persimilis” or “P. persimilis” as used hereinafter refers to a predatory mite population comprising the Phytoseiulus persimilis (P. persimilis). Phytoseiulus is a genus of mites in the Phytoseiidae family. This predatory mite is the mite predator most frequently used to control two-spotted spider mites in greenhouses and outdoor crops grown in mild environments.

P. persimilis is generally used for spider mite control and management. They are voracious predators of most of the spider mite pests (Tetranychus spp). Some of the species they impact include: the two-spotted mite Tetranychus urticae, the carmine red mite T. cinnabarinus, and the Pacific mite T. pacificus. Unlike Neoseiulus californicus (Order: Mesostigmata, Family: Phytoseiidae, Subfamily: Amblyseiinae) which may not eat for relatively long periods, Phytoseiulus persimilis must have fresh feed. Furthermore, according to existing knowledge Phytoseiulus persimilis are not flexible by their diet as other available predatory mite species for spider mite control, since they are known to only feed upon specific Tetranychus species, but not all of them.

The present invention discloses for the first time succesfull reproduction of P. persimilis on non-Tetranychid arthropod prey, in particular selected from the order Astigmata and from the immobilized Phytoseiidae. On the basis of the surprising finding that Phytoseiulus species, contrary to the general conviction in the art, may be reproduce on non-Tetranychid arthropod prey, new rearing systems for Phytoseiulus species may be developed. Such rearing systems of the present invention are much more cost effective than rearing P. persimilis on its conventional diet which consists of phytophagous mites.

The term “factitious host” generally refers hereinafter to an unnatural host or host other than the target host for the predatory mite, one that biocontrol practitioners may more readily rear than the target host in a laboratory. In the context of the present invention, factitious host or prey refers to organisms unlikely to be attacked by a natural enemy or predatory mite in its natural habitat, but that is artificially used to support its development and/or reproduction. Usually it is a species that is easier and less expensive to rear. Examples within the scope of the present invention include storage mites (such as astigmatid mites) for predatory mites (such as Phytoseiulus mite species), mite eggs for predatory insects and mites. According to further aspects, the term factitious host is used when a biological control agent is forced to feed on an insect or mite that it would not feed on it in nature. This can allow higher production levels. The present invention shows for the first time that species of commercially available Phytoseiulus mites can be mass reared using astigmatid mites (Acari: Astigmata) as factitious prey.

The term “juvenile mite” or “juvenile mites” refers hereinafter to mite developmental life stages or mite developmental phases or instar including egg, larva, protonymph and deutonymph (third instar) individuals.

The term “individual” or “individuals” or “mite individuals” refers in the context of the present invention to mite developmental stages including, but not limited to eggs, juvenile mite stages such as larva, protonymph and deutonymph (third instar) individuals.

The term “mobile stages” refers hereinafter to mite developmental stages including larva, protonymph, deutonymph (third instar) and adult stages.

The term “immobilized” used hereinafter generally means that the non-tetranychid arthropod prey individuals, preferably Astigmatid individuals, have been subjected to an immobilization treatment. An immobilization treatment should be construed to mean a treatment which impairs the motility that a prey individual has in any of its life stages (including immobile stages, i.e. eggs and any mobile developmental stage). Motility being the capability of moving spontaneously and independently. As the skilled person is aware of, life stages of mites which are motile are larvae, nymphs and adults. Thus treatments that impair the motility of any of these stages should be considered to be an immobilization treatment. In addition treatments that prevent individuals to develop from a non-motile life stage, such as from the egg stage to a motile life stage, should also be considered an immobilization treatment. According to a preferred embodiment the immobilized mite individuals comprise eggs, larvae, nymphs or adults, preferably lives stages comprising eggs, most preferably eggs combined with juvenile live stages. According to a further preferred embodiment the prey individuals are permanently immobilized. A treatment rendering the prey individuals, preferably Astigmatid mites, “non-viable” (i.e causing death) may be considered a permanently immobilizing treatment. According to some embodiments of the present invention the immobilized, preferably non viable mite individuals are produced by or exposed to a treatment including, but not limited to, thermal treatment, such as freezing, freeze-drying, heating, cold-shock or heat-shock treatment; chemical treatment, such as gas or fume treatment; radiation treatment, such as UV, microwave, gamma irradiation or X-ray treatment; mechanical treatment, such as vigorous shaking, or stirring, subjecting to shear forces, collision; gas pressure treatment, such as ultrasound treatment, pressure changes, pressure drops; electrical treatment, such as electrocution; immobilizing with an adhesive; immobilization by starvation, such as induced by water or food deprivation; immobilization by suffocation or anoxia treatment, such as by temporarily eliminating oxygen from the atmosphere or replacing oxygen by another gas and any combination thereof. WO2013/103294 further discloses immobilized Astigmatid mites and methods for obtaining them. As the non-tetranychod prey, non-hatching eggs of Astigmatid mites (e g immobilized by freezing or by radiation treatment), more preferably in combination with immobilized, in particular non-viable, juvenile Astigmatid live stages are most preferred in the context of the present invention.

The term “non-viable” used hereinafter generally means not capable of living, growing, developing, or functioning. According to main aspects of the present invention it refers to dead or not alive or non-living or immobilized mites (i.e. any mite developmental stage or phase) or mite eggs. In a specific embodiment of the present invention, non-viable Astigmata mites and/or eggs are used as a prey for predatory mites of the Phytoseiulus genus.

According to a specific embodiment, the composition of the present invention comprises C. lactis eggs and/or mites and/or larvae, immobilized by freezing, used as a prey for predatory mites of the Phytoseiulus genus. According to an aspect of the invention, the eggs mites and/or larvae may be non-viable or dead.

The term “Astigmatid” or “Astigmata” or “Astigmatic mites” or “Astigmatina” as used herein refers to mites order within the Subclass: Acari. The Astigmatina are a “cohort” of mites. Astigmatina belongs to the Sarcoptiformes, which contains the “biting” Acariformes. The Astigmata order contains superfamilies with over thousands of genera. Non limiting examples of such superfamilies and families, within the scope of the present invention may include:

Suborder: Acaridia

Superfamilies:

Schizoglyphoidea: examples of families include: Schizoglyphidae

Histiostomatoidea: examples of families include: Histiostomatidae, Guanolichidae

Canestrinioidea: examples of families include: Chetochelacaridae, Lophonotacaridae, Canestriniidae, Heterocoptidae

Hemisarcoptoidea: examples of families include: Chaetodactylidae, Hyadesiidae,

Carpoglyphidae, Algophagidae, Hemisarcoptidae, Winterschmidtiidae

Glycyphagoidea: examples of families include: Euglycyphagidae, Chortoglyphidae, Pedetropodidae, Echimyopodidae, Aeroglyphidae, Rosensteiniidae, Glycyphagidae

Acaroidea: examples of families include: Sapracaridae, Suidasiidae, Lardoglyphidae, Glycacaridae, Gaudiellidae

Acaridae: examples of families include: Hypoderoidea, Hypoderidae

Suborder: Psoroptidia

Superfamilies:

Pterolichoidea: examples of families include: Oconnoriidae, Ptiloxenidae

Pterolichidae: examples of families include: Cheylabididae, Ochrolichidae,

Gabuciniidae, Falculiferidae, Eustathiidae, Crypturoptidae, Thoracosathesidae, Rectijanuidae, Ascouracaridae, Syringobiidae, Kiwilichidae, Kramerellidae

Freyanoidea: examples of families include: Freyanidae, Vexillariidae, Caudiferidae

Analgoidea: examples of families include: Heteropsoridae, Analgidae, Xolalgidae, Avenzoariidae, Pteronyssidae, Proctophyllodidae, Psoroptoididae, Trouessartiidae, Alloptidae, Thysanocercidae, Dermationidae, Epidermoptidae, Apionacaridae, Dermoglyphidae, Laminosioptidae, Knemidokoptidae, Cytoditidae

Pyroglyphoidea: examples of families include: Pyroglyphidae, Turbinoptidae

Psoroptoidea: examples of families include: Psoroptidae, Galagalgidae, Lobalgidae, Myocoptidae, Rhyncoptidae, Audycoptidae, Listrophoridae, Chirodiscidae, Atopomelidae, Chirorhynchobiidae, Gastronyssidae, Lemurnyssidae, Pneumocoptidae, Sarcoptidae.

The claims further present Astigmatid species suitable as the non-Tetranychid arthropod prey in embodiments of the different aspects of the invention. According to many embodiments of the different aspects of the invention, selection of a non-Tetranychid arthropod prey from an Astigmatid species is most preferred. When used as a food source for the Phytoseiulus species, Astigmatid individuals are most preferably used in immobilized form, in particular in an immobilized form having immobilized life stages comprising immobilized (non-hatching) eggs Immobilization by freezing is in particular suitable and is the most preferred method of immobilization for Astigmatid individuals Immobilization by irradiation treatment is an alternative highly favourable immobilization method.

A preferable Astigmatid mite species used by the biological control system of the present invention as a factitious host population for the Phytoseiulus predatory mite, e.g. P. persimilis, is a mite species of the Carpoglyphidae family, more preferably Carpoglyphus lactis (C. lactis).

Carpoglyphidae is a mite family in the order Astigmatina, containing four genera: Carpoglyphus, Coproglyphus, Dichotomiopus and Pullea.

Carpoglyphus lactis (Acarus lactis), is most preferably used by the present invention as a diet for rearing P. persimilis, belongs to the Carpoglyphus genus. Carpoglyphus lactis is acknowledged herein as a stored product mite, infesting saccharide-rich stored commodities including dried fruits, wine, beer, milk products, jams and honey. Since C. lactis is capable of feeding on stored products, it is highly desirable and cost effective to raise P. persimilis on this mite species, as shown for the first time by the present invention. When used as a food source for the Phytoseiulus species, Carpoglyphus lactis individuals are most preferably used in immobilized form, in particular in an immobilized form having immobilized life stages comprising immobilized (non-hatching) eggs (and/or immobilized mites) Immobilization by freezing is in particular suitable and the most preferred method of immobilization for Carpoglyphus lactis.

In a further embodiment of the different aspects of the present invention, the Phytoseiulus predatory mite, e.g. P. persimilis, can complete its life cycle and reproduce when feeding on immobilized, in particular non-viable mites and/or eggs of the species Carpoglyphus lactis and/or Dermatophagoides firinae both belonging to the Astigmata order.

The term “trait” refers hereinafter to characteristic or phenotype. A phenotypic trait may refer to the appearance or other detectable characteristic of an individual, resulting from the interaction of its genome, proteome and/or metabolome with the environment. For example, in the context of the present invention an increased reproduction rate as described herein is a phenotypical trait characterizing the predatory mites of the composition of the present invention. According to a further embodiment of the present invention, a trait may also arise from interaction between the mite and its associated microorganisms. A trait may be inherited in a dominant or recessive manner, or in a partial or incomplete-dominant manner. A trait may be monogenic (i.e. determined by a single locus) or polygenic (i.e. determined by more than one locus) or may also result from the interaction of one or more genes with the environment. A dominant trait results in a complete phenotypic manifestation at heterozygous or homozygous state; conventionally, a recessive trait manifests itself only when present at homozygous state.

The term “genetic linkage” is understood within the scope of the invention to refer to an association of characters in inheritance due to location of genes in proximity on the same chromosome, measured by percent recombination between loci (centi-Morgan, cM).

As used herein, the term “population” refers to a plurality of individuals. According to some embodiments the term includes a genetically heterogeneous collection of mites sharing a common genetic derivation.

In the context of the current invention, two distinct Phytoseiulus species populations, e.g. P. persimilis populations are herein disclosed. A population, designated as P+, reproduced and selected on a non-tetranychid arthropod prey, preferably on an immobilized non-tetranychid arthropod prey, such as on a non-phytophagous prey, preferably on an Astigmatid prey, most preferably on an immobilized Astigmatid prey, such as an immobilized Astigmatid prey, in particular a Carpoglyhus prey, having immobilized life stages comprising immobilized eggs. In some aspects of the invention, the P+ population is characterized by improved reproduction on the non-tetranychid arthropod prey, defined herein by parameters such as daily reproduction rate, daily oviposition rate, female and/or juvenile survival rate and percentage of female individuals capable of reproducing on the on the non-tetranychid arthropod prey.

The second population (designated P−) is a population reared on its natural host, namely tetranychid arthropod prey, or spider mites as the sole food source. The P-population is also referred to as the conventional or commercially available Phytoseiulus or P. persimilis population, or conventionally reared population or non-selected population or control population, a Phytoseiulus population comprising a fraction of female individuals capable of reproduction on immobilized Astigmatid prey having immobilized life stages comprising immobilized eggs, lower than 10% or a population of a Phytoseiulus that was available up until the current invention.

As used herein, the phrase “genetic marker” or “molecular marker” or “biomarker” refers to a feature in an individual's genome e.g., a nucleotide or a polynucleotide sequence that is associated with one or more loci or trait of interest In some embodiments, a genetic marker is polymorphic in a population of interest, or the locus occupied by the polymorphism, depending on context. Genetic markers or molecular markers include, for example, single nucleotide polymorphisms (SNPs), indels (i.e. insertions deletions), simple sequence repeats (SSRs), restriction fragment length polymorphisms (RFLPs), random amplified polymorphic DNAs (RAFDs), cleaved amplified polymorphic sequence (CAPS) markers, Diversity Arrays Technology (DArT) markers, and amplified fragment length polymorphisms (AFLPs) or combinations thereof, among many other examples such as the DNA sequence per se. Genetic markers can, for example, be used to locate genetic loci containing alleles on a chromosome that contribute to variability of phenotypic traits. The phrase “genetic marker” or “molecular marker” or “biomarker” can also refer to a polynucleotide sequence complementary or corresponding to a genomic sequence, such as a sequence of a nucleic acid used as a probe or primer.

A genetic marker can be physically located in a position on a chromosome that is within or outside of the genetic locus with which it is associated (i.e., is intragenic or extragenic, respectively).

As used herein, the term “germplasm” refers to the totality of the genotypes of a population or other group of individuals (e.g., a species).

The terms “hybrid” and “hybrid progeny” used herein refers to an individual produced from genetically different parents (e.g., a genetically heterozygous or mostly heterozygous individual).

The term “allele(s)” used herein means any of one or more alternative or variant forms of a gene or a genetic unit at a particular locus, all of which alleles relate to one trait or characteristic at a specific locus. In a diploid cell of an organism, alleles of a given gene are located at a specific location, or locus (loci in plural) on a chromosome. One allele is present on each chromosome of the pair of homologous chromosomes. A diploid plant species may comprise a large number of different alleles at a particular locus. Such alternative or variant forms of alleles may be the result of single nucleotide polymorphisms, insertions, indels, inversions, translocations or deletions, or the consequence of gene regulation caused by, for example, chemical or structural modification, transcription regulation or post-translationalmodification/regulation. An allele associated with a qualitative trait may comprise alternative or variant forms of various genetic units including those that are identical or associated with a single gene or multiple genes or their products or even a gene disrupting or controlled by a genetic factor contributing to the phenotype represented by the locus.

As used herein, the term “locus” means a specific place or places or region or a site on a chromosome where for example a gene or genetic marker element or factor is found. In specific embodiments, such a genetic element is contributing to a trait.

As used herein, the term “breeding” and grammatical variants thereof, refer to any process that generates a progeny individual. Breeding can be sexual or asexual, or any combination thereof. Exemplary non-limiting types of breeding include crossing, introgressing, selfing, backcrossing, doubled haploid derivative generation, and combinations thereof.

The term “genetic determinant” as used herein refers to genetic determinants such as genes, alleles, QTLs or traits.

Introgression of a genetic determinant means the incorporation of genes, alleles, QTLs or traits into a line wherein essentially all of the desired morphological and physiological characteristics of the line are recovered, in addition to the genetically introgressed determinant. Such a process is often used in cultivar development, in which one or a few genetic determinants are transferred to a desired genetic background, preferably by using backcrossing.

The term “genotype” refers to the genetic constitution of a cell or organism. An individual's genotype includes the specific alleles, for one or more genetic marker loci, present in the individual's haplotype. As is known in the art, a genotype can relate to a single locus or to multiple loci, whether the loci are related or unrelated and/or are linked or unlinked. In some embodiments, an individual's genotype relates to one or more genes that are related in that the one or more of the genes are involved in the expression of a phenotype of interest. Thus, in some embodiments a genotype comprises a summary of one or more alleles present within an individual at one or more genetic loci. In some embodiments, a genotype is expressed in terms of a haplotype.

According to a further embodiment of the present invention, the Phytoseiulus predatory mite, e.g. P. persimilis, can complete its life cycle and reproduce (i.e. including development and oviposition) for at least 3 generations, when feeding on living juvenile mites of the species Amblyseius swirskii that belongs to the Phytoseiidae family.

It is further within the scope of the present invention to disclose a population of Phytoseiulus predatory mites, e.g. the mite species P. persimilis, reared by feeding on dead or immobilized mite species selected from the group comprising Carpoglyphus lactis, Dermatophagoides farinae, Lepidogyphus destructor, Glyciphagus domesticus, Dermatophagoides pteronisinus, Amblyseius swirskii, and any combination thereof.

According to a further embodiment, the predatory mite fed on the above prey mites, developed and reproduced for at least two generations.

According to a further embodiment of the present invention, P. persimilis or other Phytoseiulus predatory mite, can develop on immobilized, in particular by freezing, individuals of the following species belonging to the Astigmata order: Carpoglyphus lactis, Lepidoglyphus destructor, Glycifagus domesticus and Dermatophagoides pteronisinus.

It is further within the scope that the mites used as prey are immobilized by immobilization treatment selected from the group consisting of: thermal treatment, such as freezing, heating, cold-shock or heat-shock treatment; chemical treatment, such as gas or fume treatment; radiation treatment, such as Gamma irradiation, UV, microwave or X-ray treatment; mechanical treatment, such as vigorous shaking, or stirring, subjecting to shear forces, collision; gas pressure treatment, such as ultrasound treatment, pressure changes, pressure drops; electrical treatment, such as electrocution; immobilizing with an adhesive; immobilization by starvation, such as induced by water or food deprivation; immobilization by suffocation or anoxia treatment, such as by temporarily eliminating oxygen from the atmosphere or replacing oxygen by another gas.

The skilled person will understand how these treatments may result in the immobilization of the Astigmatid individuals or other mites of the Phytoseiidae family and that the immobilization treatment should be such that the mite individuals remain a suitable prey (food source) for the predatory mite individuals.

It is further within the scope that the term “immobilized mites” may also mean dead or non-living mites.

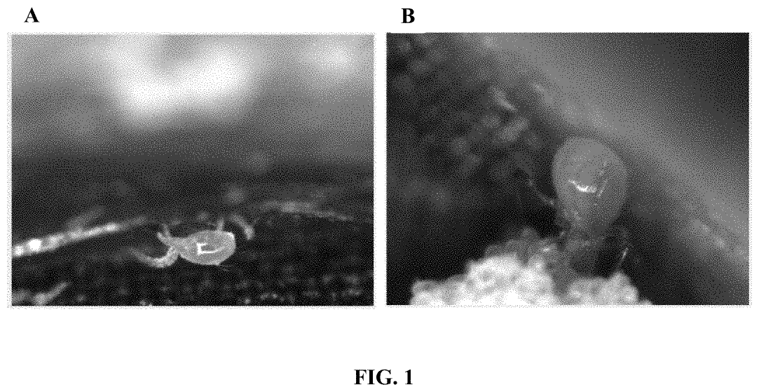

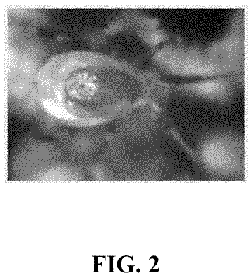

Reference is now made to FIG. 1 photographically presenting different developmental stages of P. persimilis reared on dead or immobilized C. lactis. The figure presents an adult female (FIG. 1A) and a juvenile that had just hatched from the egg (FIG. 1B). As can be seen in this figure, all stages are characterized by a pale whitish color, typical to this diet, in contrast to the normal orange color obtained when feeding P. persimilis by spider mites, their conventional diet. In other words, the predators of the present invention, fed on C. lactis, turn to beige-white instead of the typical orange color. In addition, the dorsal shield of the predator is darker than the cuticle around it. This figure demonstrates that P. persimilis can develop and reproduce on dead or immobilized C. lactis mites. As explained above, C. lactis (Acari: Astigmata) are significantly more cost effective to produce than the conventional P. persimilis diet, which is the phytophagous spider mite.

Reference is now made to FIG. 2 photographically presenting P. persimilis reared on dead or immobilized C. lactis. As can be seen, the predator has a unique appearance, where it turns to beige-white instead of the typical orange (when fed on conventional spider mite diet) and the dorsal shield of the predator is darker than the cuticle around it.

It is herein acknowledged that twospotted spider mites feed on many species of plants and are a major pest of vegetables, ornamentals, fruit trees, hops, cotton, and strawberries (van de Vrie et al., 1972). At present, it can be assumed that most of the major spider mite problems in greenhouses will involve twospotted spider mite. The larva, protonymph, deutonymph, and adult feed mainly on the undersides of the leaves.

It is within the scope of the present invention to provide a composition for controlling mite pests, particularly members of the Acari class, family Tetranychidae such as twospotted spider mite, more particularly spider mite species, especially the genera Tetranychus, Panonychus and various other mite species.

According to some embodiments of the present invention, the crop is selected from the group consisting of greenhouse grown crops and open field crops. Non limiting examples of crop types within the scope of the present invention include vegetables, ornamentals, fruit trees, hops, cotton and strawberries.

Specific examples of mite pests- host plant species within the scope of the present invention include the following:

Acanthaceae: Acanthus mollis; Justicia adhatoda.

Actinidiaceae: Actinidia chinensis; Actinidia deliciosa; Actinidia sp.

Adoxaceae: Sambucus canadensis; Sambucus chinensis; Sambucus edulus; Sambucus nigra; Sambucus sieboldiana; Sambucus sp.; Viburnum lantana; Viburnum opulus; Viburnum rhytidophyllum; Viburnum sp.; Viburnum tinus.

Aizoaceae: Mesembryanthemum crystallinum.

Alstroemeriaceae: Alstroemeria sp.

Amaranthaceae: Alternanthera sp.; Amaranthus blitum; Amaranthus caudatus; Amaranthus graecizans; Amaranthus hybridus; Amaranthus mangostanus; Amaranthus palmeri; Amaranthus retroflexus; Amaranthus sp.; Amaranthus spinosus; Amaranthus viridis; Atriplex canescens; Atriplex lentiformis; Atriplex semibaccata; Beta vulgaris; Celosia argentea; Chenopodium album; Chenopodium murale; Chenopodium sp.; Dysphania ambrosioides; Haloxylon ammodendron; Iresine herbstii; Salsola vermiculata; Spinacia oleracea.

Amaryllidaceae: Allium ampeloprasum; Allium cepa; Allium fistulosum; Allium sativum; Allium sp.; Narcissus sp.

Anacardiaceae: Mangifera indica; Pistacia terebinthus; Pistacia vera.

Annonaceae: Annona muricata; Annona reticulata; Annona squamosa.

Apiaceae: Aegopodium podagraria; Ammi majus; Apium graveolens; Apium nodiflorum; Arracacia xanthorrhiza; Athamanta macedonica; Bupleurum lancifolium; Coriandrum sativum; Cryptotaenia canadensis; Daucus carota; Eryngium sp.; Foeniculum vulgare; Pastinaca sativa; Petroselinum crispum; Peucedanum japonicum; Phellolophium madagascariense; Spananthe sp.

Apocynaceae: Ampelamus laevis; Apocynum cannabinum; Asclepias sp.; Catharanthus roseus; Mandevilla sp.; Matelea carolinensis; Nerium oleander; Plumeria sp.; Raphionacme sp.; Rauvolfia serpentina; Vinca major; Vinca sp.

Aquifoliaceae: Ilex crenata.

Araceae: Alocasia macrorrhizos; Alocasia sp.; Anthurium sp.; Arumitalicum; Arum sp.; Caladium bicolor; Caladium sp.; Calla sp.; Colocasia esculenta; Colocasia sp.; Dieffenbachia sp.; Epipremnum pinnatum; Philodendron sp.; Symplocarpus fbetidus; Xanthosoma sp.; Zantedeschia aethiopica.

Araliaceae: Aralia sp.; Hedera canariensis; Hedera helix; Hedera sp.; Hydrocotyle umbellata; Polyscias balfouriana; Schefflera actinophylla; Schefflera elegantissima; Schefflera sp.; Tetrapanax papyrifer.

Araucariaceae: Agathis sp.; Araucaria sp.

Arecaceae: Dypsis sp.; Phoenix dactylifera; Phoenix sp.; Veitchia sp.

Aristolochiaceae: Aristolochia clematitis.

Asparagaceae: Asparagus laricinus; Asparagus officinalis; Asparagus setaceus; Asparagus sp.; Aspidistra elation; Cordyline fruticosa; Cordyline sp.; Dracaena braunii; Dracaena fragrans; Dracaena goldieana; Dracaena sp.; Hyacinthus orientalist Lachenalia ensifolia; Maianthemum racemosum; Ornithogalum sp.; Polygonatum odoratum; Ruscus aculeatus; Yucca sp.

Balsaminaceae: Impatiens balsamina; Impatiens sp.; Impatiens walleriana.

Berberidaceae: Berberis cretica; Berberis thunbergii; Berberis vulgaris; Berberis wilsoniae; Nandina domestica.

Betulaceae: Alnus incana; Betula maximowicziana; Betula papyrifera; Betula pendular Carpinus sp.; Corylus avellana.

Bignoniaceae: Campsis radicans; Pyrostegia venusta; Tecoma capensis; Tecoma stans.

Boraginaceae: Borago officinalis; Cynoglossum columnae; Heliotropium arborescens; Heliotropium eichwaldii; Heliotropium europaeum; Nama hispidum; Omphalodes verna.

Brassicaceae: Aethionema saxatile; Brassica juncea; Brassica napus; Brassica oleracea; Brassica rapa; Brassica sp.; Capsella bursa pastoris; Diplotaxis erucoides; Diplotaxis viminea; Eruca vesicaria; Erysimum graecum; Erysimum sp.; Erysimum x cheiri; Hirschfeldia incana; Lepidium didymum; Malcolmia sp.; Matthiola fruticulosa; Matthiola incana; Matthiola odoratissima; Nasturtium sp.; Raphanus raphanistrum; Raphanus sp.; Rapistrum rugosum; Rorippa indica; Sinapis arvensis; Zilla spinosa.

Bromeliaceae: Tillandsia sp.

Buxaceae: Buxus sempervirens.

Calophyllaceae: Mammea Americana.

Campanulaceae: Campanula erinus; Lobelia sp.; Platycodon grandiflorus.

Cannabaceae: Cannabis sativa; Celtis australis; Celtis occidentalis; Humulus lupulus; Humulus scandens; Trema micrantha.

Cannaceae: Canna indica.

Capparaceae: Capparis nummularia.

Caprifoliaceae: Cephalaria gigantea; Diervilla sp.; Leycesteria Formosa; Lonicera etrusca; Lonicera nigra; Lonicera periclymenum; Lonicera sp.; Lonicera tatarica; Lonicera xylosteum; Pterocephalus plumosus; Scabiosa sicula; Symphoria racemosa; Symphoricarpos albus; Symphoricarpos orbiculatus; Weigela hortensis.

Caricaceae: Carica papaya.

Caryophyllaceae: Dianthus armeria; Dianthus barbatus; Dianthus caryophyllus; Dianthus chinensis; Dianthus sp.; Dianthus tenuiflorus; Drymaria cordata; Gypsophila paniculata; Myosoton aquaticum; Silene chalcedonica; Silene vulgaris; Stellaria media.

Celastraceae: Celastrus orbiculatus; Celastrus scandens; Euonymus europaeus; Euonymus japonicus; Euonymus sp.

Cistaceae: Helianthemum salicifolium.

Cleomaceae: Cleome sp.; Cleome viscosa.

Clethraceae: Clethra arborea.

Combretaceae: Terminalia catappa.

Commelinaceae: Commelina benghalensis; Commelina communis; Commelina diffusa.

Compositae: Acanthospermum hispidum; Achillea filipendulina; Achillea fraasii; Ageratum conyzoides; Ageratum houstonianum; Ambrosia trifida; Anthemis chia; Arctium lappa; Arctium minus; Arctotheca calendula; Arctotis sp.; Artemisia dracunculus; Bellis annua; Bidens bipinnata; Bidens biternata; Bidens pilosa; Bidens sp.; Boltonia sp.; Brachyscome sp.; Calendula arvensis; Calendula officinalis; Calendula sp.; Callistephus chinensis; Carduus crispus; Carthamus tinctorius; Centaurea cyanus; Centaurea hyalolepis; Centaurea iberica; Centaurea imperialist Centaurea montana; Chaenactis stevioides; Chrysanthemum coronarium; Chrysanthemum indicum; Chrysanthemum morifolium; Chrysanthemum segetum; Chrysanthemum sp.; Chrysothamnus viscidiflorus; Cichorium endivia; Cichorium intybus; Cichorium pumilum; Cichorium spinosum; Conyza bonariensis; Conyza canadensis; Conyza sp.; Cosmos bipinnatus; Cosmos sp.; Crassocephalum crepidioides; Crepis neglecta; Crepis rubra; Cynara cardunculus; Cynara sp.; Dahlia coccinea; Dahlia sp.; Dahlia variabilis; Elephantopus mollis; Erigeron annuus; Erigeron sp.; Euryops sp.; Euthamia graminifolia; Galinsoga caracasana; Galinsoga ciliata; Galinsoga parviflora; Gerbera jamesonii; Gerbera sp.; Helianthella quinquenervis; Helianthus annuus; Helichrysum luteoalbum; Helichrysum tenax; Helichrysum thianschanicum; Heliopsis sp.; Helminthotheca echioides; Lactuca saligna; Lactuca sativa; Lactuca serriola; Lapsana communis; Leontodon autumnalis; Leucanthemum vulgare; Melampodium perfoliatum; Melanthera aspera; Mikania micrantha; Montanoa bipinnatifida; Notobasis syriaca; Osteospermum sp.; Parthenium sp.; Pentzia globosa; Picris pauciflora; Picris sprengeriana; Pseudognaphalium obtusifolium; Rudbeckia amplexicaulis; Rudbeckia sp.; Schkuhria pinnata; Scolymus maculatus; Scorzonera sp.; Senecio lividus; Senecio sp.; Senecio vulgaris; Solidago gigantea; Sonchus arvensis; Sonchus aspen; Sonchus oleraceus; Sonchus sp.; Tagetes erecta; Tagetes microglossa; Tagetes minuta; Tagetes patula; Tagetes sp.; Taraxacum officinale; Tithonia rotundifolia; Tragopogon dubius; Tragopogon pratensis; Tridax procumbens; Urospermum dalechampii; Vernonia sp.; Xanthium strumarium; Zinnia elegans; Zinnia sp. Convolvulaceae: Calystegia hederacea; Calystegia sepium; Convolvulaceae sp.; Convolvulus arvensis; Convolvulus hirsutus; Convolvulus scammonia; Convolvulus siculus; Convolvulus sp.; Convolvulus tricolor; Dinetus racemosus; Ipomoea aquatica; Ipomoea arachnosperma; Ipomoea batatas; Ipomoea biflora; Ipomoea cairica; Ipomoea hochstetteri; Ipomoea indica; Ipomoea lacunosa; Ipomoea lobata; Ipomoea nil; Ipomoea purpurea; Ipomoea sp.; Ipomoea tricolor; Ipomoea triloba.

Cornaceae: Cornus alba; Cornus canadensis; Cornus nuttallii; Cornus sp.

Cucurbitaceae: Benincasa hispida; Bryonia alba; Citrullus colocynthis; Citrullus lanatus; Cucumis melo; Cucumis sativus; Cucumis sp.; Cucurbita ficifolia; Cucurbita maxima; Cucurbita moschata; Cucurbita pepo; Cucurbita sp.; Cucurbitaceae sp.; Diplocyclos palmatus; Ecballium elaterium; Lagenaria siceraria; Luffa acutangula; Luffa cylindrica; Momordica charantia; Praecitrullus fistulosus; Sechium edule.

Cupressaceae: Chamaecyparis thyoides; Cupressus sp.; Juniperus arizonica; Juniperus virginiana; Platycladus orientalis.

Cyperaceae: Cyperus esculentus; Cyperus rotundus; Cyperus schimperianus.

Dipterocarpaceae: Shorea robusta.

Ebenaceae: Diospyros kaki; Diospyros scabrida.

Elaeagnaceae: Elaeagnus angustifolia; Elaeagnus umbellata.

Equisetaceae: Equisetum palustre.

Ericaceae: Azalea nudiflora; Azalea sp.; Rhododendron sp.; Siphonandra sp.

Euphorbiaceae: Acalypha australis; Acalypha havanensis; Acalypha sp.; Acalypha wrilkesiana; Codiaeum sp.; Codiaeum variegatum; Croton niveus; Croton sp.; Euphorbia amygdaloides; Euphorbia burmanni; Euphorbia helenae; Euphorbia helioscopia; Euphorbia hirta; Euphorbia hypericifolia; Euphorbia parviflora; Euphorbia pulcherrima; Euphorbia sp.; Hevea brasiliensis; Hura crepitans; Jatropha gossypiifolia; Jatropha hastata; Jatropha multifida; Jatropha sp.; Manihot esculenta; Manihot sp.; Mercurialis annua; Mercurialis sp.; Ricinus communis.

Fagaceae: Quercus alba; Quercus robur; Quercus sp.

Garryaceae: Aucuba japonica.

Gentianaceae: Eustoma grandiflorum; Gentiana sp.

Geraniaceae: Erodium alnifolium; Geranium carolinianum; Geranium dissectum; Geranium lucidum; Geranium molle; Geranium rotundifolium; Geranium sp.; Pelargonium inquinans; Pelargonium sp.

Gesneriaceae: Saintpaulia ionantha.

Goodeniaceae: Goodenia sp.; Scaevola sp.

Grossulariaceae: Ribes americanum; Ribes nigrum; Ribes rubrum .

Heliconiaceae: Heliconia bihai; Heliconia latispatha.

Hydrangeaceae: Deutzia sp.; Hydrangea macrophylla; Hydrangea paniculata; Hydrangea sp.; Philadelphus coronarius; Philadelphus sericanthus.

Iridaceae: Crocosmia x crocosmiiflora; Gladiolus hortulanus; Gladiolus italicus; Gladiolus sp.; Iris sanguinea; Iris x germanica; Ixia flexuosa.

Juglandaceae: Carya illinoinensis; Juglans regia; Juglans sp.

Lamiaceae: Ajuga sp.; Ballota africana; Clerodendrum chinense; Clerodendrum thomsoniae; Galeopsis speciosa; Galeopsis tetrahit, Glechoma hederacea; Glechoma sp.; Holmskioldia sanguinea; Holmskioldia sp.; Lamium album; Lamium amplexicaule; Lamium purpureum; Lamium sp.; Lavandula sp.; Leonotis ocymifolia; Leucas martinicensis; Marrubium vulgare; Melissa officinalis; Mentha arvensis; Mentha sp.; Mentha spicata; Mentha x piperita; Moluccella laevis; Monarda fistulosa; Nepeta cataria; Ocimum basilicum; Ocimum tenuiflorum; Perilla frutescens; Rosmarinus officinalis; Salvia argentea; Salvia officinalis; Salvia pratensis; Salvia sp.; Salvia splendens; Salvia verticillata; Salvia viridis; Stachys arvensis; Vitex negundo.

Lauraceae: Cassytha sp.; Endlicheria paniculata; Laurus nobilis; Persea americana.

Leguminosae: Acacia greggii; Acacia horrida; Acacia huarango; Acacia karroo; Acacia robusta; Acacia sp.; Alysicarpus longifolius; Amphicarpaea bracteata; Anthyllis vulneraria; Arachis hypogaea; Arachis sp.; Astragalus sinicus; Bauhinia forficata; Bauhinia monandra; Bauhinia sp.; Bauhinia variegata; Bituminaria bituminosa; Canavalia ensiformis; Caragana arborescens; Cassia artemisioides; Ceratonia siliqua; Cercis siliquastrum; Cicer arietinum; Clianthus sp.; Clitoria ternatea; Coronilla valentina; Crotalaria juncea; Crotalaria micans; Crotalaria sp.; Dalbergia sissoo; Dalea mollis; Desmodium khasianurn; Dolichos sp.; Erythrina corallodendron; Erythrina poeppigiana; Erythrina sp.; Genista sp.; Gleditsia sp.; Glycine max; Indigofera arrecta; Indigofera holubii; Indigofera tinctoria; Inga sp.; Kennedia coccinea; Lablab purpureus; Laburnum anagyroides; Laburnum sp.; Lathyrus cicera; Lathyrus odoratus; Lathyrus sativus; Lens culinaris; Lespedeza maximowiczii; Lotus corniculatus; Lupinus arboreus; Lupinus argenteus; Lupinus sativus; Macroptilium atropurpureum; Macroptilium lathyroides; Medicago arabica; Medicago arborea; Medicago lupulina; Medicago orbicularis; Medicago polymorpha; Medicago sativa; Medicago sp.; Melilotus albus; Melilotus indicus; Melilotus sp.; Mucuna membranacea; Mucuna pruriens; Neonotonia wightii; Neorautanenia mitis; Onobrychis viciifolia; Ornithopus sp.; Phaseolus acutifolius; Phaseolus coccineus; Phaseolus lunatus; Phaseolus sp.; Phaseolus vulgaris; Pisum sativum; Psophocarpus tetragonolobus; Pueraria montana; Pueraria phaseoloides; Rhynchosia capitata; Rhynchosia caribaea; Robinia hispida; Robinia pseudoacacia; Sesbania cannabina; Sesbania herbacea; Spartium junceum; Styphnolobium japonicum; Teramnus uncinatus; Tipuana tipu; Trifolium alexandrinum; Trifolium aureum; Trifolium dasyurum; Trifolium dubium; Trifolium glomeratum; Trifolium hybridum; Trifolium incarnatum; Trifolium pratense; Trifolium purpureum; Trifolium repens; Trifolium sp.; Trifolium spumosum; Vicia angustifolia; Vicia faba; Vicia pulchella; Vicia sativa; Vicia sp.; Vicia villosa; Vigna aconitifolia; Vigna angularis; Vigna mungo; Vigna radiata; Vigna sp.; Vigna unguiculata; Wisteria floribunda; Wisteria polystachya; Wisteria sinensis.

Liliaceae: Lilium sp.

Linaceae: Reinwardtia tetragyna.

Lythraceae: Cuphea sp.; Lagerstroemia speciosa; Punica granatum.

Magnoliaceae: Magnolia liliiflora; Magnolia sp.; Magnolia stellata.

Malvaceae: Abelmoschus esculentus; Abutilon pictum; Abutilon reflexum; Abutilon sp.; Abutilon theophrasti; Abutilon tubulosum; Alcea rosea; Althaea nudiflora; Byttneria australis; Ceiba pentandra; Corchorus capsularis; Corchorus olitorius; Gossypium barbadense; Gossypium herbaceum; Gossypium hirsutum; Gossypium sp.; Grewia asiatica; Grewia bilobo; Helicteres guazumifolia; Hibiscus lunariifolius; Hibiscus mutabilis; Hibiscus rosa-sinensis; Hibiscus sp.; Hibiscus syriacus; Hibiscus trionurn; Malva aegyptia; Malva moschata; Malva neglecta; Malva nicaeensis; Malva parviflora; Malva sp.; Malva sylvestris; Malva trimestris; Malvella leprosa; Sida rhombifolia; Sida sp.; Sterculia murex; Tilia americana; Tilia cordata; Tilia platyphyllos; Tilia rubra; Tilia sp.; Tilia tomentosa; Tilia x euchlora; Triumfetta semitriloba; Waltheria indica.

Marantaceae: Calathea sp.; Maranta sp.

Meliaceae: Azadirachta indica; Melia azedarach; Toona ciliata.

Menispermaceae: Tinospora fragosa.

Moraceae: Artocarpus altilis; Ficus carica; Ficus elastica; Ficus religiosa; Ficus sp.; Morus alba; Morus nigra; Morus rubra; Morus sp.

Moringaceae: Moringa oleifera.

Musaceae: Musa acuminata; Musa basjoo; Musa sp.; Musa x paradisiaca.

Myrtaceae: Eucalyptus grandis; Psidium cattleianum; Psidium guajava; Syzygium cumini.

Nothofagaceae: Nothofagus alpina.

Nyctaginaceae: Bougainvillea spectabilis.

Olacaceae: Ximenia americana.

Oleaceae: Forsythia koreana; Forsythia suspensa; Forsythia x intermedia; Fraxinus angustifolia; Fraxinus excelsior; Fraxinus ornus; Fraxinus sp.; Jasminum humile; Jasminum nudiflorum; Jasminum officinale; Jasminum sambac; Jasminum sp.; Ligustrum lucidum; Ligustrum vulgare; Olea europaea; Osmanthus fragrans; Syringa oblata; Syringa vulgaris.

Onagraceae: Chylismia claviformis; Epilobium angustifolium; Fuchsia magellanica; Fuchsia sp.; Fuchsia x hybrida; Gaura sp.; Oenothera biennis; Oenothera laciniata; Oenothera sp.; Oenothera tetraptera.

Orchidaceae: Catasetum sp.; Cymbidium sp.; Orchidaceae sp.; Papilionanthe teres.

Oxalidaceae: Oxalis corniculata; Oxalis debilis; Oxalis europaea; Oxalis floribunda; Oxalis sp.

Papaveraceae: Argemone mexicana; Bocconia frutescens; Chelidonium majus; Chelidonium sp.; Dicentra sp.; Eschscholzia sp.; Fumaria officinalis; Papaver aculeatum; Papaver nudicaule; Papaver orientale; Papaver rhoeas; Papaver somniferum

Passifloraceae: Passiflora caerulea; Passiflora edulis; Passiflora fbetida; Passiflora mollissima; Passiflora sp.

Paulowniaceae: Paulownia fortunei.

Pedaliaceae: Sesamum indicum.

Phyllanthaceae: Phyllanthus amarus; Phyllanthus sp.

Phytolaccaceae: Petiveria alliacea; Phytolacca americana; Phytolacca dioica; Phytolacca esculenta; Phytolacca icosandra.

Pinaceae: Pinus sylvestris; Tsuga canadensis.

Pittosporaceae: Pittosporum tobira.

Plantaginaceae: Angelonia sp.; Antirrhinum majus; Digitalis purpurea; Hippuris vulgaris; Linaria genistifolia; Mecardonia procumbens; Plantago asiatica; Plantago lanceolata; Plantago major; Plantago sp.; Veronica persica; Veronica sp.; Veronica teucrium

Platanaceae: Platanus orientalis; Platanus sp.

Plumbaginaceae: Limoniastru guyonianum; Limonium sinuatum; Plumbago auriculata; Plumbago sp.

Poaceae: Aegilops sp.; Agropyron desertorum; Aira sp.; Avena fatua; Avena sativa; Avena sp.; Avena sterilis; Bambusa sp.; Bromus catharticus; Bromus sp.; Chondrosum barbatum; Cynodon dactylon; Dactyloctenium aegyptium; Digitaria argillacea; Digitaria ciliaris; Digitaria diversinervis; Digitaria sanguinalis; Eleusine coracana; Elymus hispidus; Elymus repens; Eragrostis sp.; Festuca arundinacea; Festuca sp.; Helictotrichon pratense; Hordeum sp.; Lolium multiflorum; Lolium sp.; Ophiuros exaltatus; Oryza glaberrima; Oryza sativa; Panicum miliaceum; Panicum sp.; Paspalum dilatatum; Pennisetum clandestinum; Pennisetum purpureum; Phleum pratense; Poa annua; Poa pratensis; Poa trivialis; Poaceae sp.; Rottboellia cochinchinensis; Saccharum officinarum; Setaria pumila; Setaria viridis; Sitanion hystrix; Sorghum bicolor; Sorghum halepense; Sorghum sp.; Stenotaphrum secundatum; Triticum sp.; Zea mays; Zeugites sp.

Polemoniaceae: Phlox carolina; Phlox paniculata; Phlox sp.

Polygonaceae: Emex australis; Fallopia baldschuanica; Fallopia convolvulus; Persicaria hydropiper; Persicaria longiseta; Persicaria maculosa; Persicaria pensylvanica; Polygonum argyrocoleon; Polygonum aviculare; Rumex acetosa; Rumex acetosella; Rumex crispus; Rumex japonicus; Rumex obtusifolius; Rumex sp.

Pontederiaceae: Eichhornia crassipes.

Portulacaceae: Portulaca oleracea.

Primulaceae: Cyclamen graecum; Cyclamen hederifolium; Cyclamen persicum; Cyclamen sp.; Primula denticulata; Primula polyantha; Primula sp.; Primula veris.

Ranunculaceae: Adonis aestivalis; Anemone coronaria; Anemone hortensis; Aquilegia sp.; Clematis paniculata; Clematis sp.; Delphinium sp.; Helleborus sp.; Ranunculus asiaticus; Thalictrum fendleri.

Resedaceae: Reseda odorata.

Rhamnaceae: Frangula dodonei; Helinus integrifolius; Rhamnus alpina; Rhamnus imeretina; Ziziphus jujuba; Ziziphus spina-christi

Rosaceae: Alchemilla vulgaris; Armeniaca mume; Cerasus lusitanica; Cerasus serrula; Cerasus vulgaris; Chaenomeles japonica; Chaenomeles sinensis; Cotoneaster horizontalis; Cotoneaster microphyllus; Cotoneaster tomentosa; Crataegus laevigata; Crataegus monogyna; Crataegus sanguinea; Cydonia oblonga; Eriobotrya japonica; Filipendula ulmaria; Fragaria moschata; Fragaria vesca; Fragaria virginiana; Fragaria x ananassa; Geum rivale; Malus domestica; Malus floribunda; Malus pumila; Malus sp.; Marcetella maderensis; Padus avium; Potentilla fragarioides; Potentilla fruticosa; Potentilla norvegica; Potentilla tanacetifolia; Prunus amygdalus; Prunus armeniaca; Prunus avium; Prunus cerasifera; Prunus cerasoides; Prunus cerasus; Prunus domestica; Prunus insititia; Prunus lusitanica; Prunus persica; Prunus salicina; Prunus serotina; Prunus sp.; Prunus spinosa; Pyracantha coccinea; Pyracantha koidzumii; Pyracantha sp.; Pyrus communis; Pyrus pyrifolia; Pyrus sp.; Rosa canina; Rosa cymosa; Rosa hybrida; Rosa multiflora; Rosa odorata; Rosa rugosa; Rosa sp.; Rosa x alba; Rosa x centifolia; Rosa x damascena; Rosa x rugosa; Rubus buergeri; Rubus chaerophyllus; Rubus chingii; Rubus fruticosus; Rubus idaeus; Rubus lloydianus; Rubus occidentalis; Rubus sp.; Rubus ulmifolius; Sorbus aucuparia; Sorbus sp.; Spiraea japonica .

Rubiaceae: Colfea arabica; Colfea sp.; Galium aparine; Galium stellatum; Gardenia jasminoides; Gardenia sp.

Rutaceae: Choisya ternata; Citrus aurantiifolia; Citrus aurantium; Citrus clementina; Citrus limon; Citrus maxima; Citrus medica; Citrus paradisi; Citrus reticulata; Citrus sinensis; Citrus sp.; Citrus trifoliata; Ruta graveolens; Zanthoxylum rhoifolium.

Salicaceae: Dovyalis cajfra; Populus alba; Populus nigra; Populus sp.; Populus tremula; Populus x canadensis; Salix aegyptiaca; Salix alba; Salix babylonica; Salix caprea; Salix chaenomeloides; Salix dephnoides; Salix fragilis; Salix sp.; Salix viminalis.

Sapindaceae: Acer campestre; Acer negundo; Acer platanoides; Acer pseudoplatanus; Acer rubrum; Acer saccharum; Acer sp.; Aesculus glabra; Dodonaea viscosa; Koelreuteria paniculata; Litchi sinensis; Sapindus sp.

Saxifragaceae: Rodgersia podophylla.

Scrophulariaceae: Buddleja davidii; Buddleja madagascariensis; Diascia sp.; Myoporum sp.; Nemesia sp.; Verbascum blattaria.

Simaroubaceae: Ailanthus altissima .

Solanaceae: Acnistus arborescens; Brugmansia arborea; Brugmansia suaveolens; Brugmansia x candida; Calibrachoa sp.; Capsicum annuum; Capsicum sp.; Cestrum cyaneum; Cestrum elegans; Cestrum strigillatum; Cyphomandra sp.; Datura metel; Datura sp.; Datura stramonium; Lycium chinense; Nicandra physalodes; Nicotiana glauca; Nicotiana sp.; Nicotiana tabacum; Petunia sp.; Petunia x hybrid, Physalis acutifolia; Physalis alkekengi; Physalis angulata; Physalis lagascae; Physalis peruviana; Salpichroa origanifolia; Solanum aethiopicum; Solanum americanum; Solanum capsicoides; Solanum carolinense; Solanum delagoense; Solanum elaeagnifolium; Solanum grandiflorum; Solanum laciniatum; Solanum lycopersicum; Solanum macrocarpon; Solanum mammosum; Solanum melongena; Solanum muricatum; Solanum nigrum; Solanum panduraeforme; Solanum quitoense; Solanum sp.; Solanum tuberosum; Withania somnifera.

Strelitziaceae: Strelitzia reginae.

Theaceae: Camellia japonica; Camellia sinensis; Camellia sp.

Thymelaeaceae: Dais cotinifolia

Tropaeolaceae: Tropaeolum majus; Tropaeolum sp.

Ulmaceae: Ulmus americana; Ulmus glabra; Ulmus laevis; Ulmus pumila; Ulmus rubra; Ulmus sp.

Urticaceae: Boehmeria nivea; Laportea aestuans; Parietaria judaica; Parietaria officinalis; Pipturus albidus; Urtica dioica; Urtica sp.; Urtica urens.

Verbenaceae: Aloysia citriodora; Duranta erecta; Glandularia phlogiflora; Lantana camara; Lippia alba; Verbena bracteata; Verbena brasiliensis; Verbena hybrida; Verbena officinalis; Verbena sp.

Violaceae: Viola odorata; Viola sp.; Viola tricolor; Viola x wittrockiana.

Vitaceae: Ampelopsis sp.; Parthenocissus quinquefolia; Parthenocissus tricuspidata; Vitis sp.; Vitis vinifera.

Xanthorrhoeaceae: Hemerocallis fulva; Hemerocallis minor.

Zingiberaceae: Curcuma longa; Zingiber mioga.

Zygophyllaceae: Tribulus terrestris.

The term “fungus reducing agent” or “fungal reducing agent” refers hereinafter to chemical fungus reducing agents such as a natural or synthetic fungicide, or to a biological fungus reducing agent such as a population of a mite species producing antifungal exudates, or a population of mycophagous mites, in particular selected from the Astigmata, for example populations of living Carpoglyphus lactis or Lepidoglyphus destructor individuals. Such fungus reducing mite populations are disclosed in WO2013/103294.

It is within the scope of the current invention that the rearing composition as defined in any of the above is absent of or is lacking a fungus reducing agent. The claimed Phytoseiulus mites of the present invention are capable of completing their life cycle and reproducing for at least 2 generations when reared upon immobilized Astigmata individuals including mites at any developmental stage and/or eggs. It is noted that non-viable Astigmata mite developmental stages are incapable of producing or secreting a fungus reducing agent.

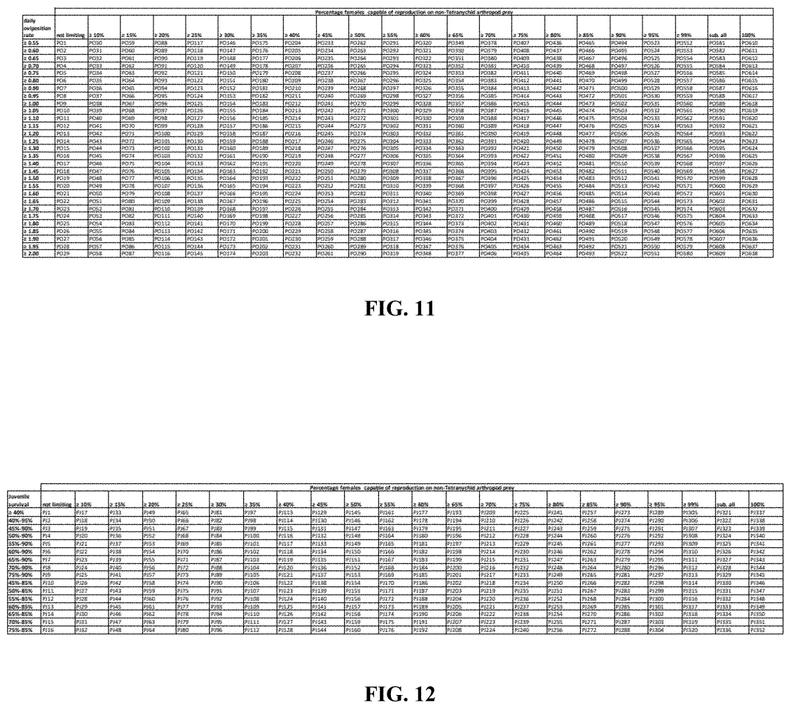

Reference is now made to FIGS. 11-17 presenting combinations of features particularly envisaged for embodiments of the different aspects of the invention. The numbers indicated provide a reference number for a particular combination of features.

FIG. 11 presents combinations of the percentage of females capable of reproducing on the non-Tetranychid arthropod prey (P) combined with values for the daily oviposition rate (O) as particularly envisaged for use in the different aspects of the present invention. The numbers indicated (PO1-PO638) provide a reference number for a particular combination of (P) and (O) values that correspond with the values at the intersection of the (P) and (O) values, where the reference number is positioned. When a reference number is positioned at the intersection of a (P) and (O) value, the combination of (P) and (O) values are thus envisaged for use within the various aspects of the invention.

In FIG. 12, similarly to FIG. 11, combinations of the percentage of females capable of reproducing on the non-Tetranychid arthropod prey (P) are combined with values for the percentage juvenile survival (J) as particularly envisaged for use in the different aspects of the present invention are presented. The numbers indicated (PJ1-PJ352) provide a reference number for a particular combination of (P) and (J) values that correspond with the values at the intersection of the (P) and (J) values where the reference number is positioned. When a reference number is positioned at the intersection of a (P) and (J) value, the combination of (P) and (J) values are thus envisaged for use within the various aspects of the invention. In the figure, the term “not limiting” means that in the indicated embodiment, the percentage female capable of reproducing on the non-Tetranychid arthropod prey, is not a limiting feature. Thus this feature can have any value and thus need not be specified or (explicitly) referred to. In the figure, “sub. all” means substantially all.

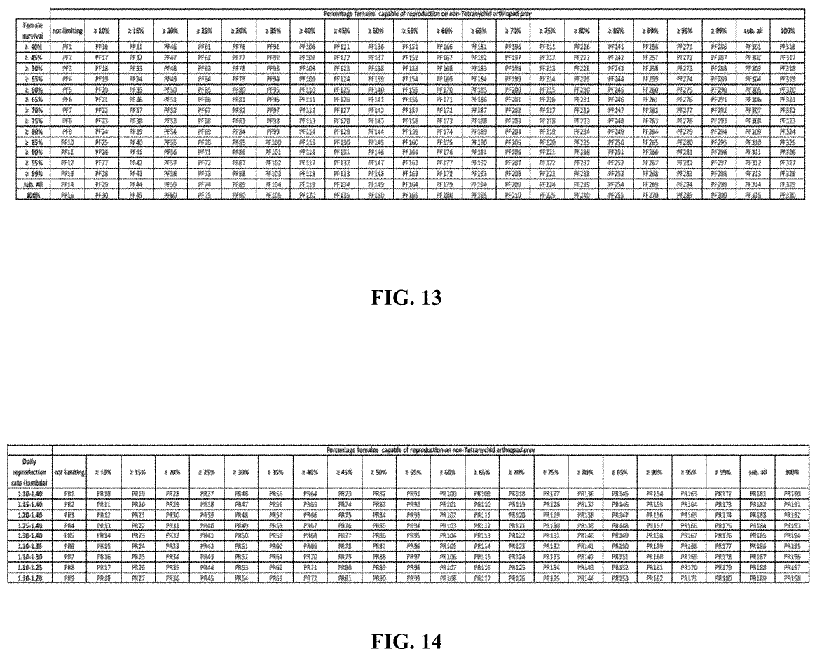

In FIG. 13, similarly to FIG. 11, combinations of the percentage of females capable of reproducing on the non-Tetranychid arthropod prey (P) are combined with values for the percentage female survival (F) as particularly envisaged for use in the different aspects of the present invention. The numbers indicated (PF1-PF330) provide a reference number for a particular combination of (P) and (F) values that correspond with the values at the intersection of the (P) and (F) values where the reference number is positioned. When a reference number is positioned at the intersection of a (P) and (F) value, the combination of (P) and (F) values are thus envisaged for use within the various aspects of the invention. In the figure, “not limiting” means that in the indicated embodiments, the percentage female capable of reproducing on the non-Tetranychid arthropod prey is not a limiting feature. Thus this feature can have any value and thus need not be specified or (explicitly) referred to. In the figure, “sub. all” means substantially all .

In FIG. 14, similarly to FIG. 11, combinations of the percentage of females capable of reproducing on the non-Tetranychid arthropod prey (P) combined with values for the daily reproduction rate lambda (R) as particularly envisaged for use in the different aspects of the present invention are presented. The numbers indicated (PR1-PR198) provide a reference number for a particular combination of (P) and (R) values that correspond with the values at the intersection of the (P) and (R) values where the reference number is positioned. When a reference number is positioned at the intersection of a (P) and (R) value, the combination of (P) and (R) values are thus envisaged for use within the various aspects of the invention. In the figure, “not limiting” means that in the indicated embodiments, the percentage female capable of reproducing on the non-Tetranychid arthropod prey is not a limiting feature. Thus this feature can have any value and thus need not be specified or (explicitly) referred to. In the figure, “sub. all” means substantially all.

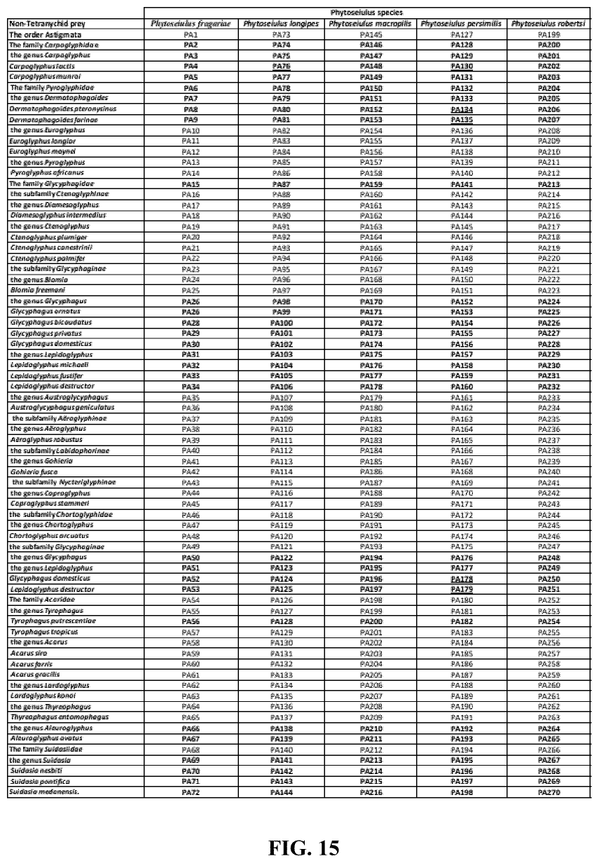

In FIG. 15, combinations of the Phytoseiulus species with (groups of) Astigmatid mites specifically envisaged for use in embodiments of the different aspects of the present invention are presented. The numbers indicated (PA1-PA270) provide a reference number for a particular combination of Phytoseiulus species and (groups of) Astigmatid mites where the reference number is at the intersection. Where in the figure a reference number is presented, the particular combination is envisaged for embodiments of the different aspects of the invention. Reference numbers presented in bold font refer to a preferred combination. Reference numbers presented in an underlined bold font refer to a more highly preferred combination.

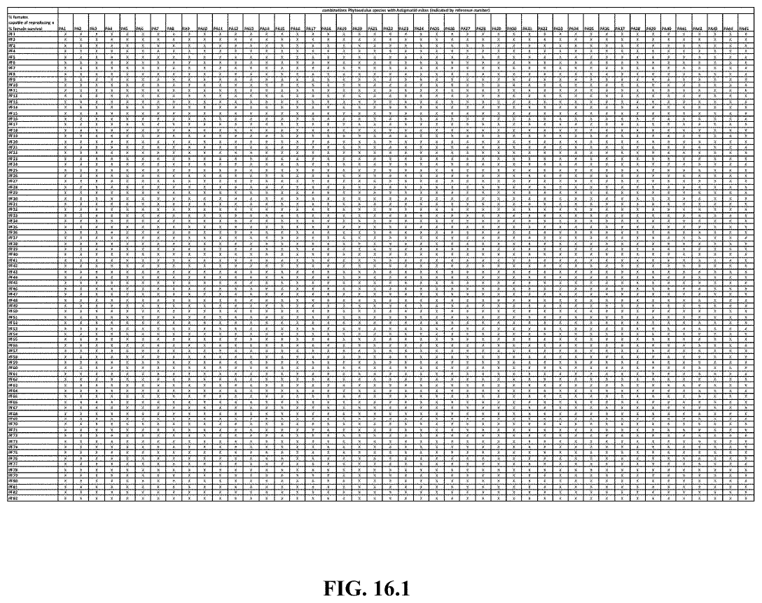

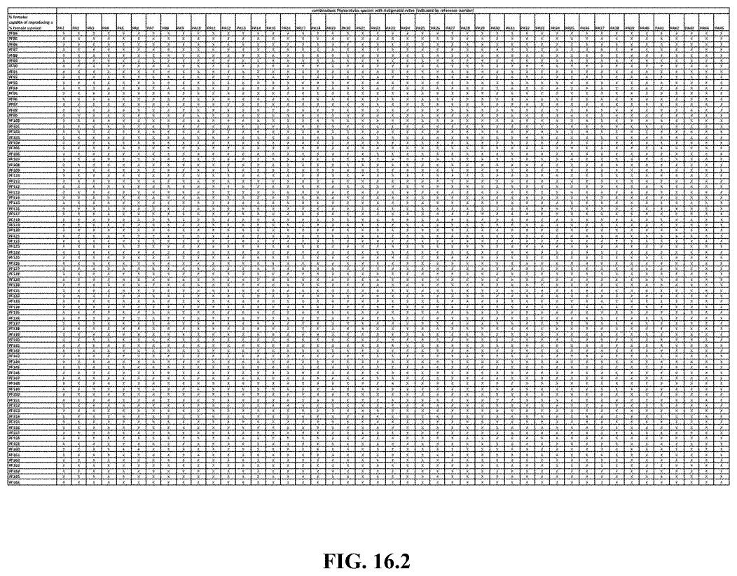

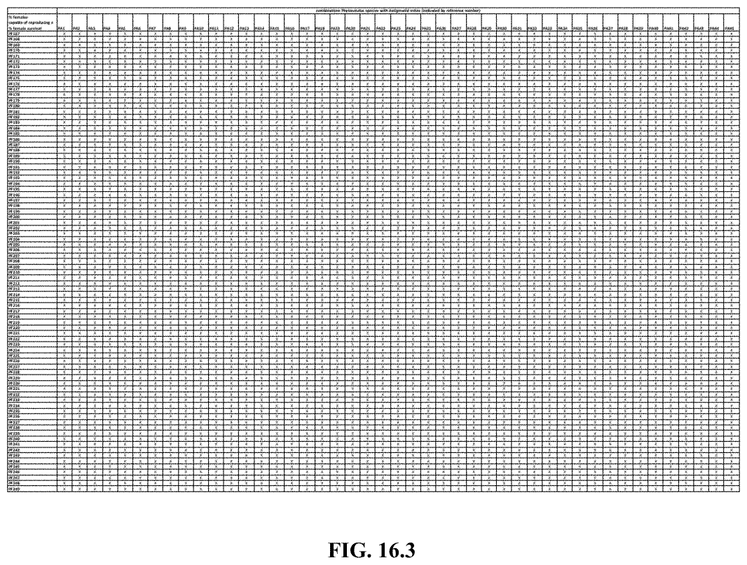

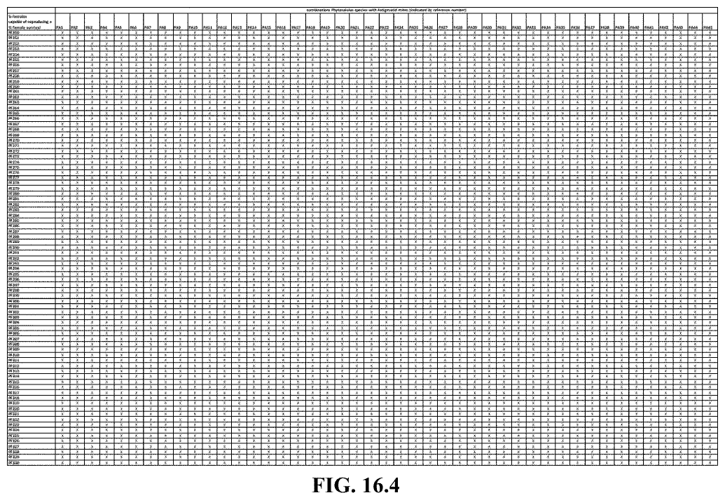

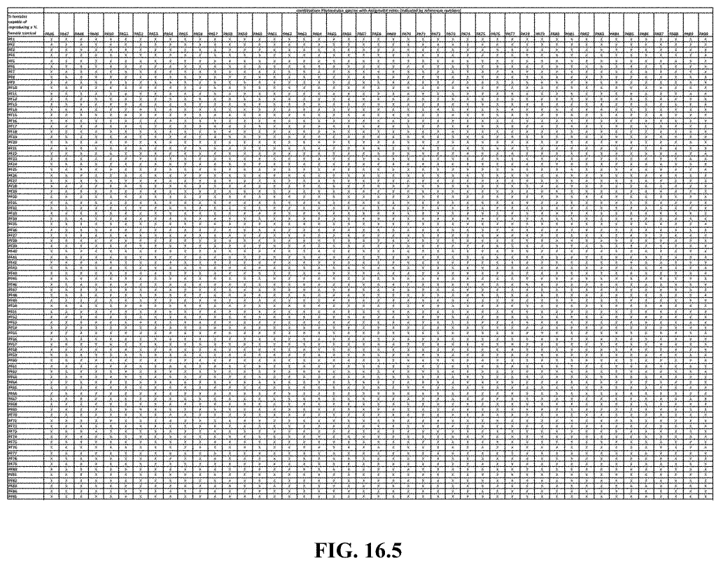

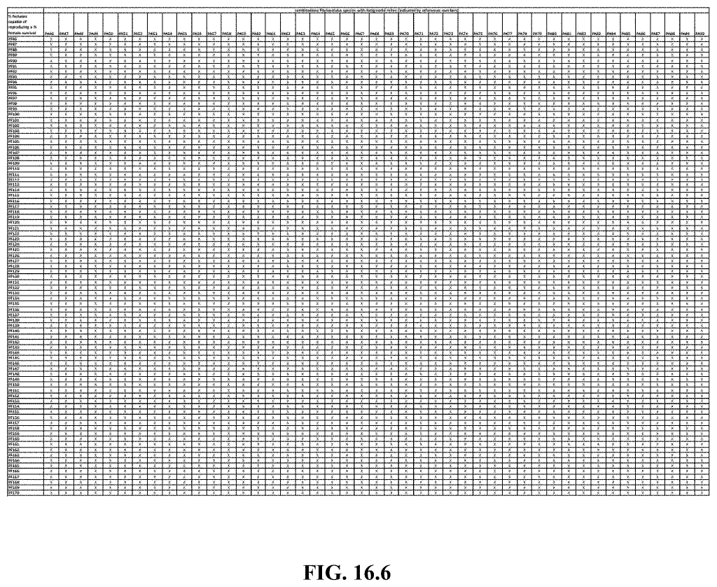

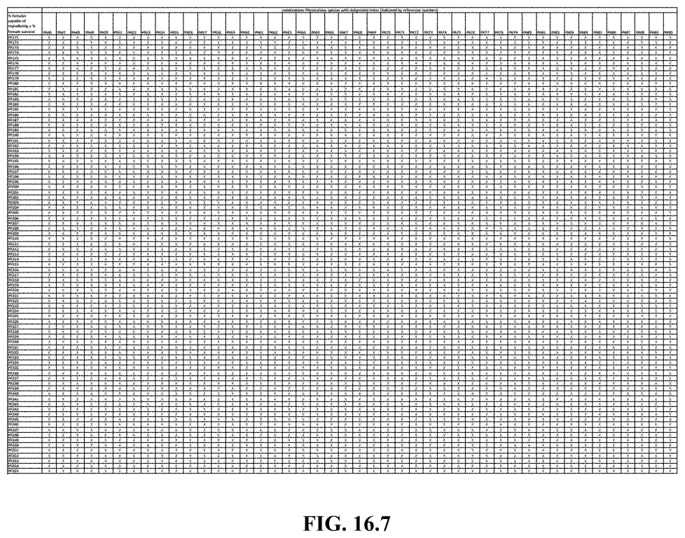

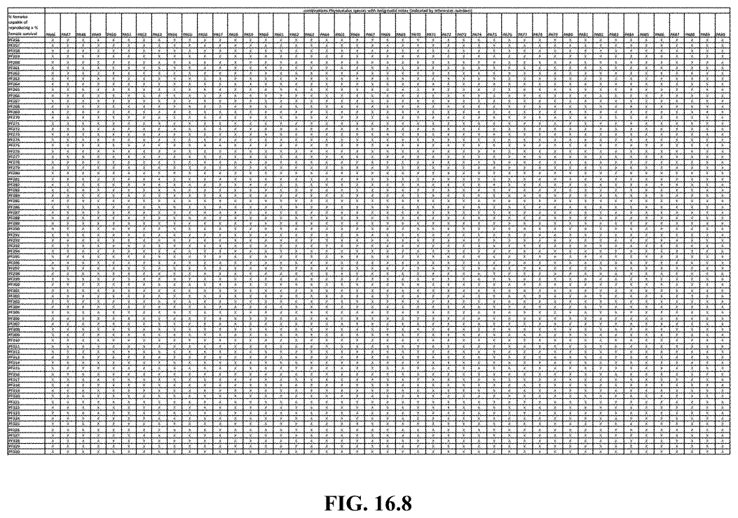

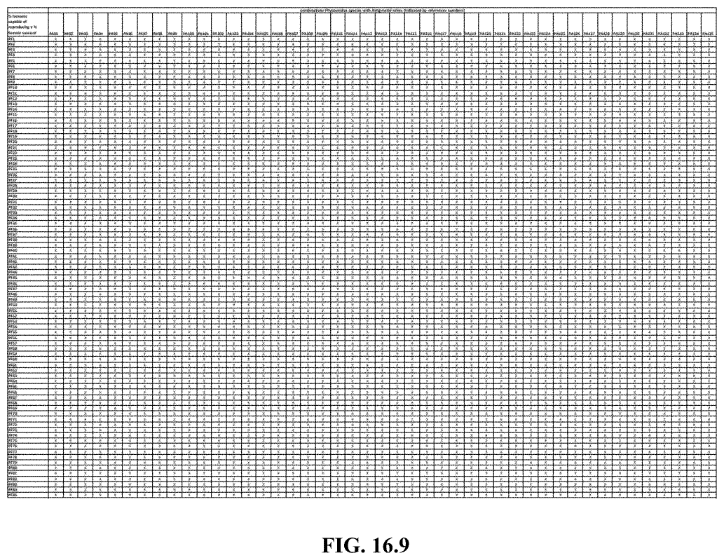

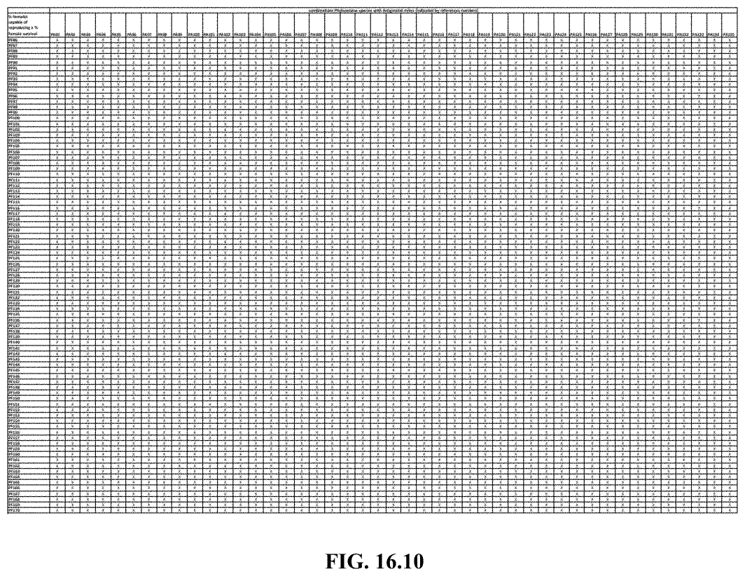

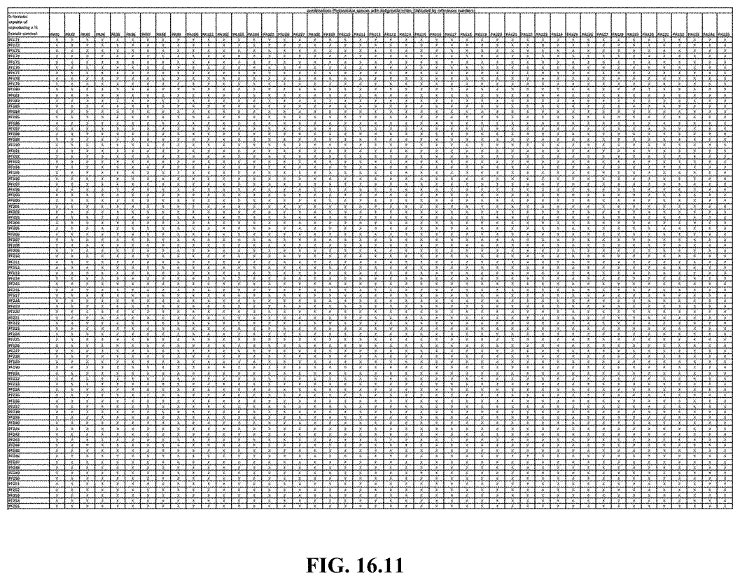

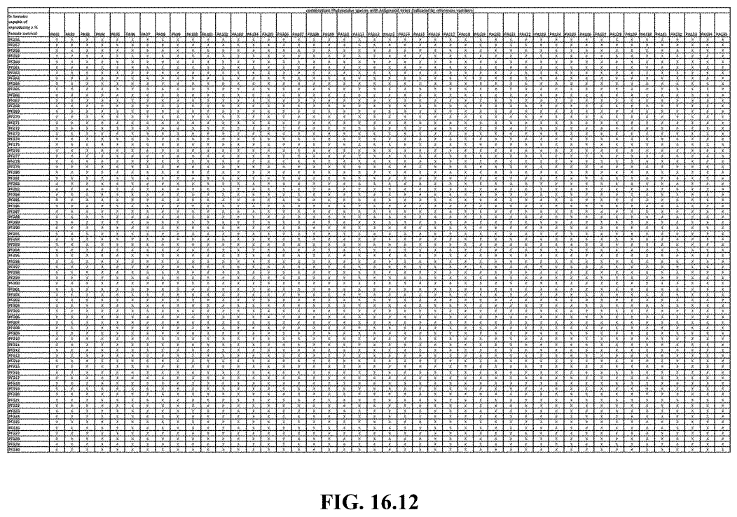

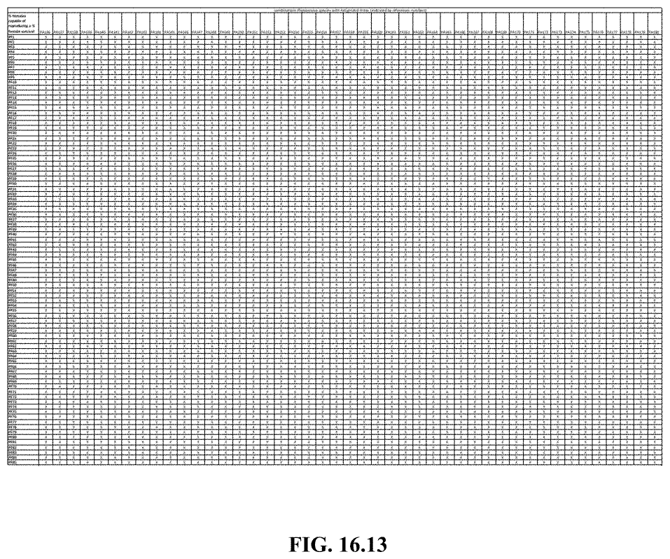

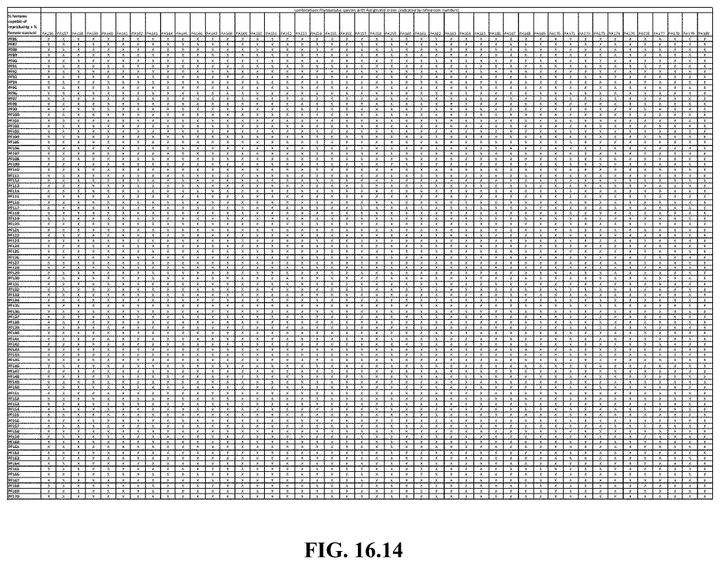

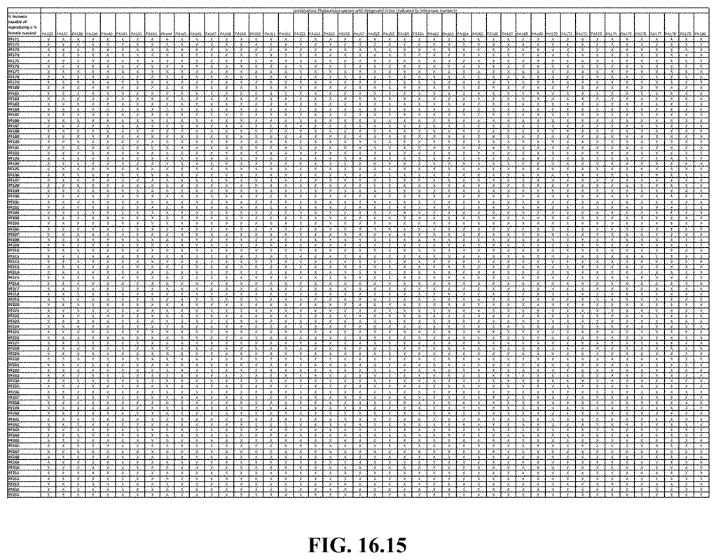

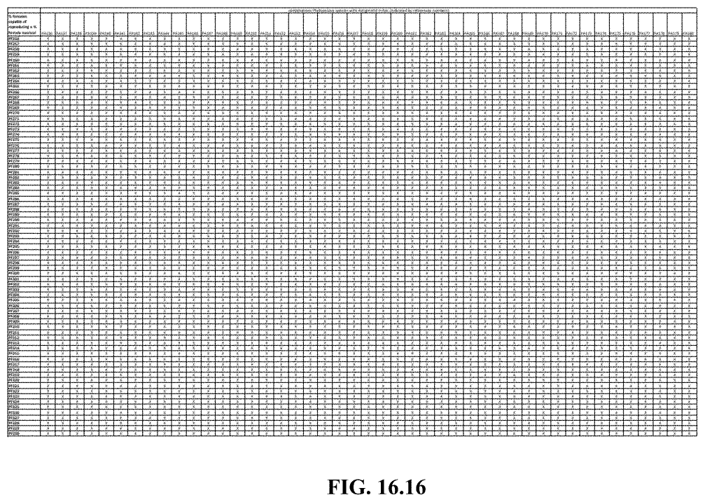

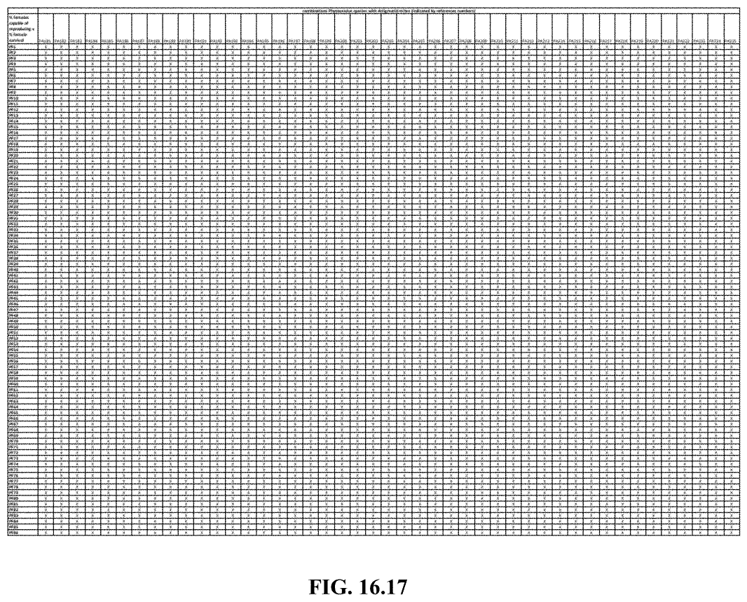

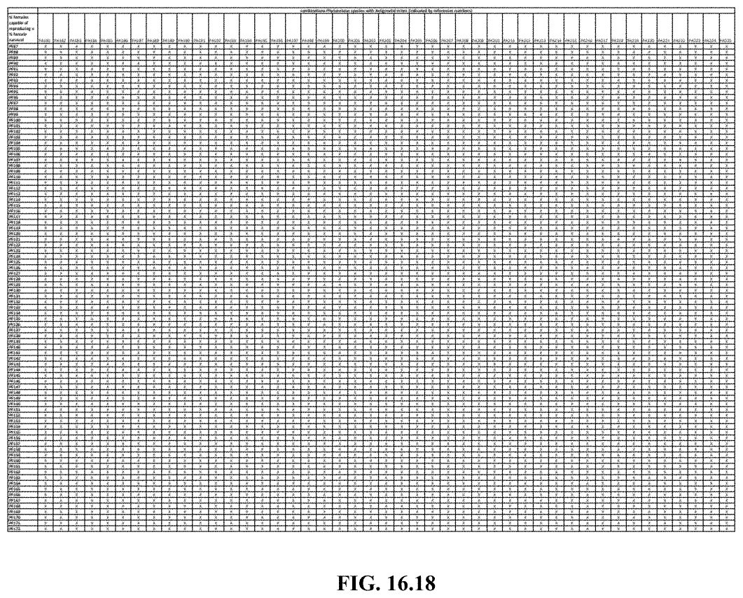

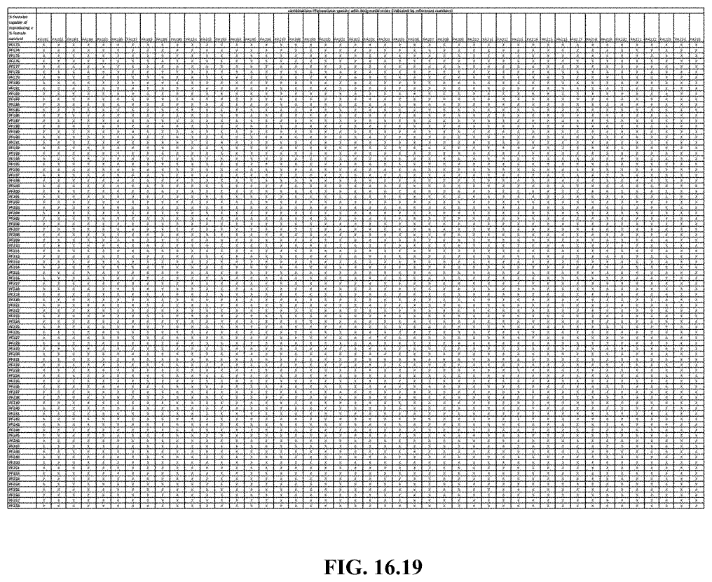

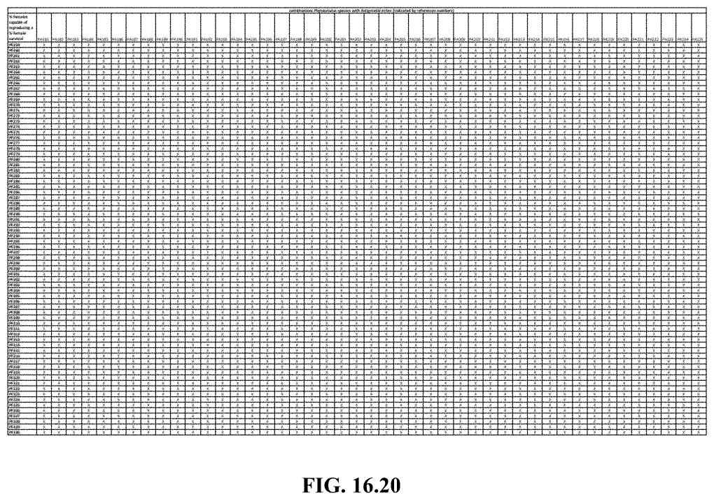

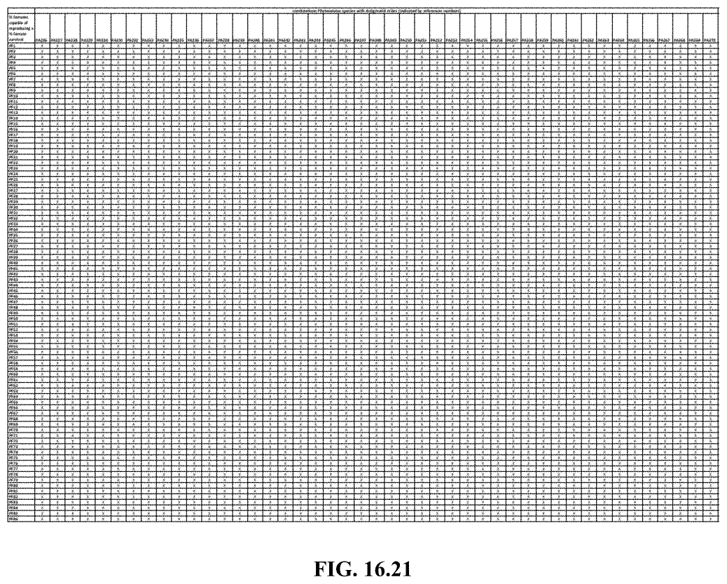

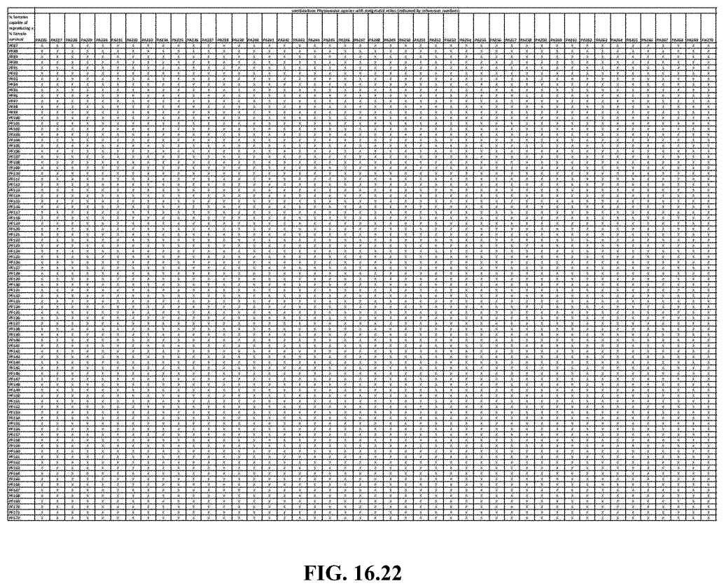

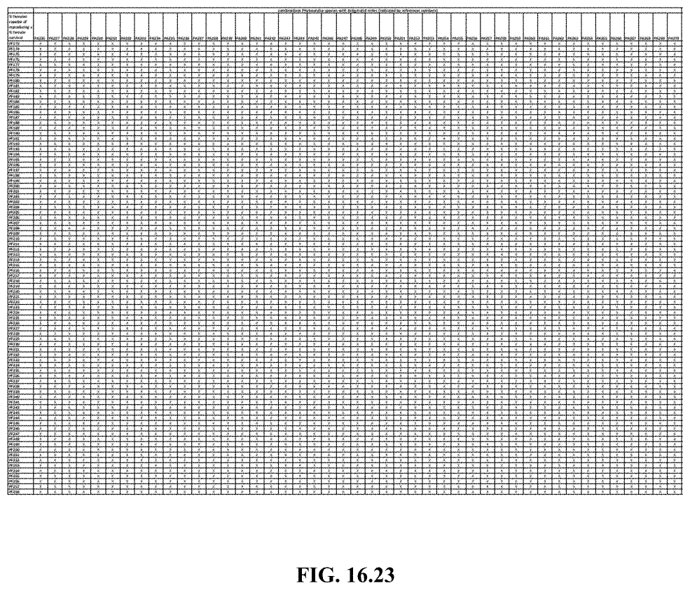

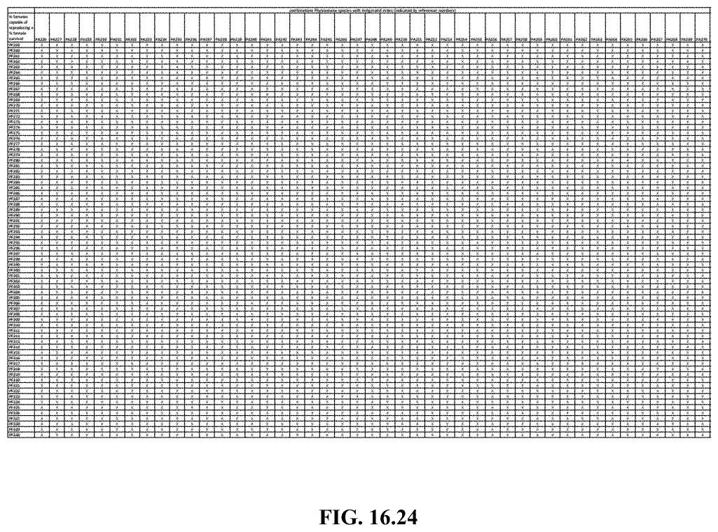

FIG. 16 presents further combinations of the combinations of the Phytoseiulus species x (groups of) Astigmatid mites (indicated by the PA1-PA270 reference numbers of FIG. 15) with the percentage of females capable of reproducing on the non-Tetranychid arthropod prey (P) x values for the percentage female survival (F) (indicated by the PF1-PF330 reference numbers FIG. 13). Where and “X” is presented, the specific combination is envisaged or applied in embodiments of the the various aspects of the present invention.

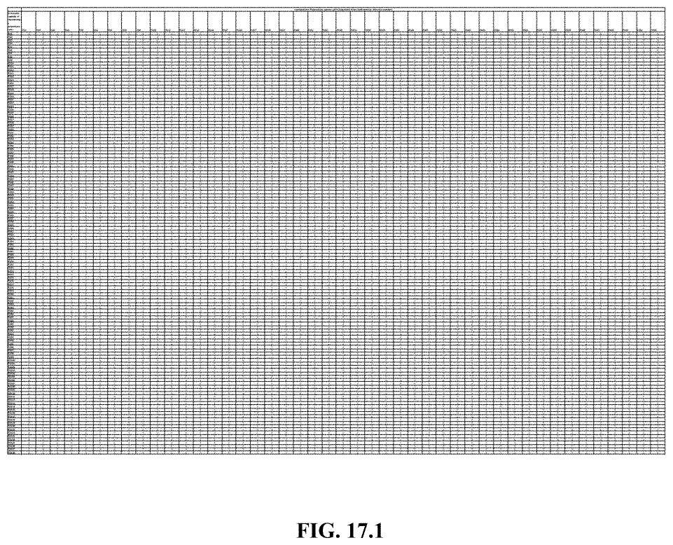

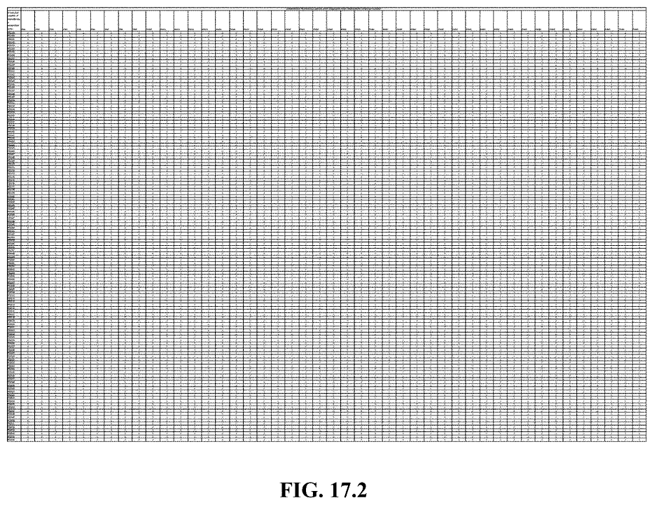

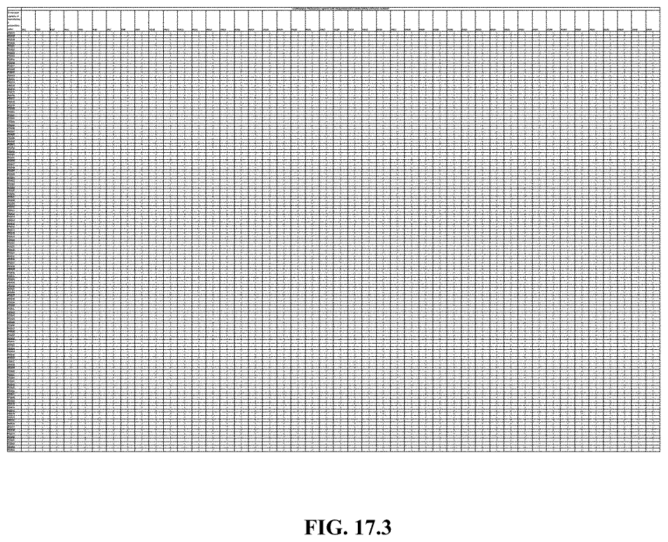

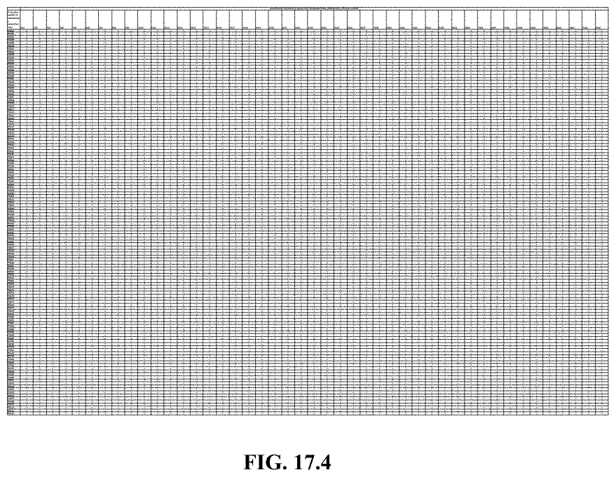

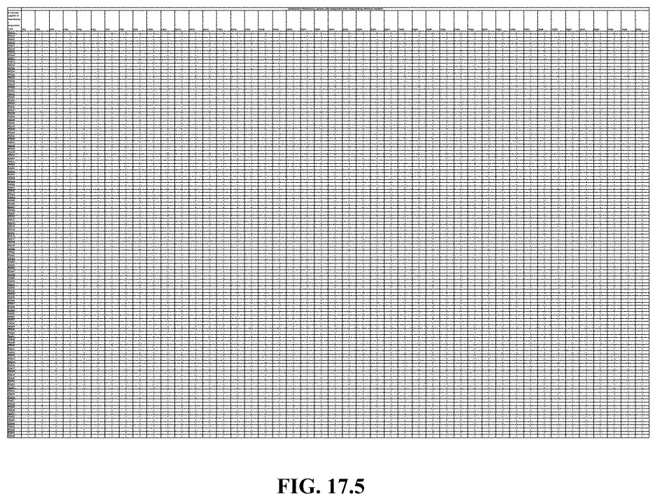

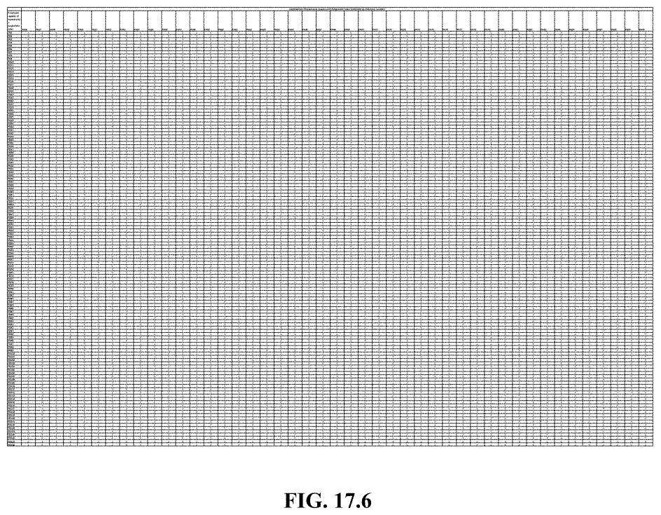

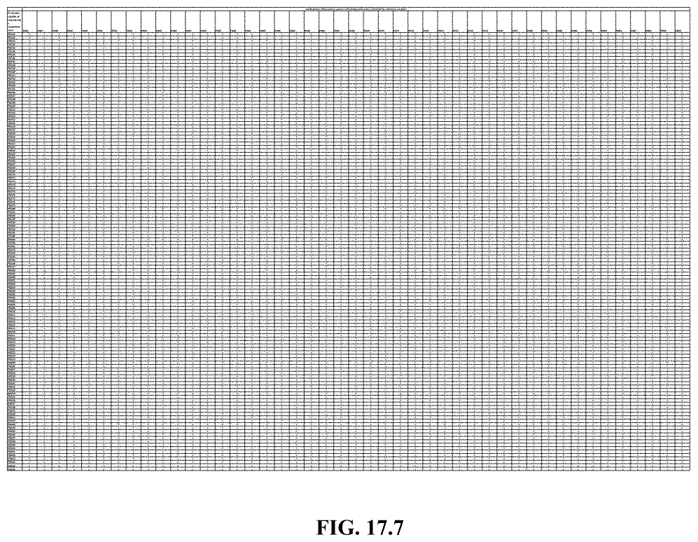

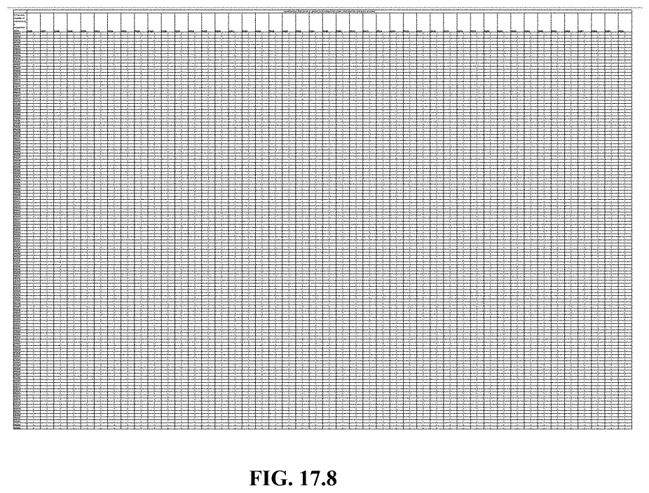

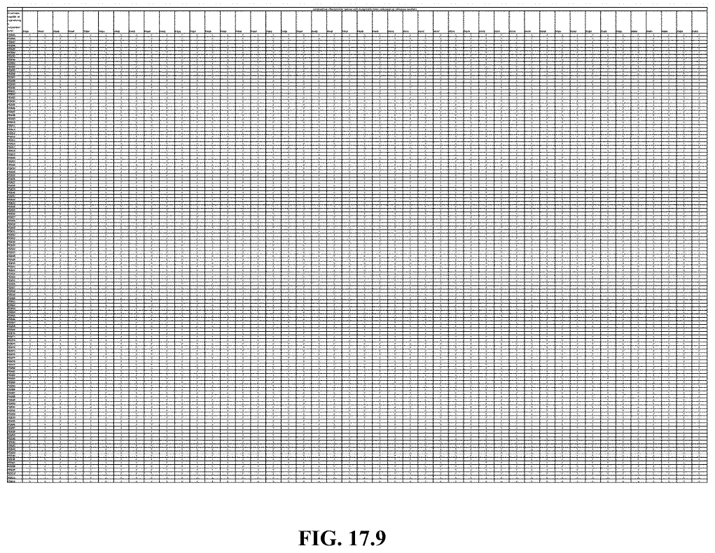

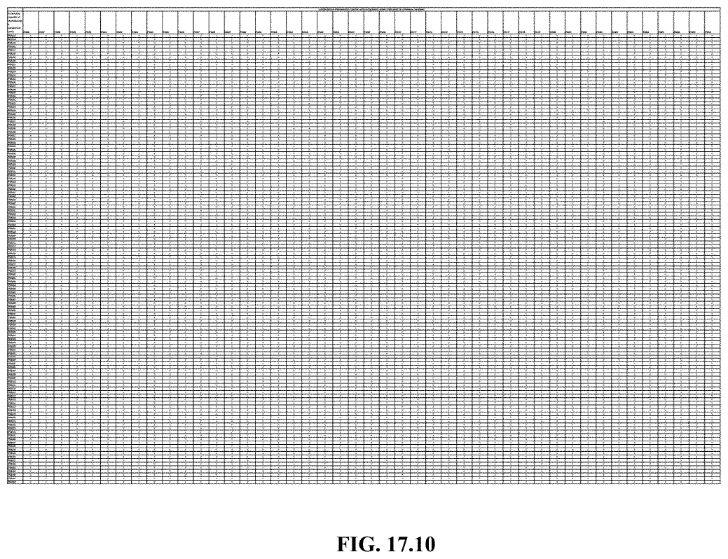

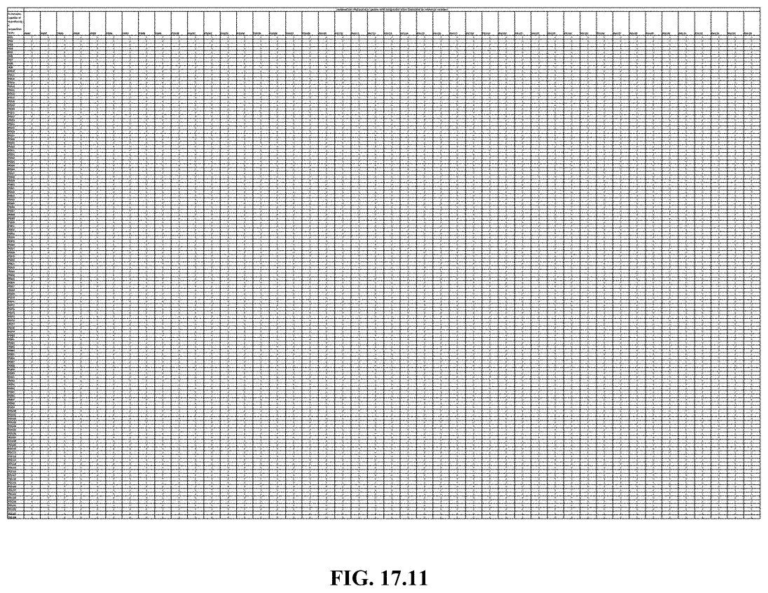

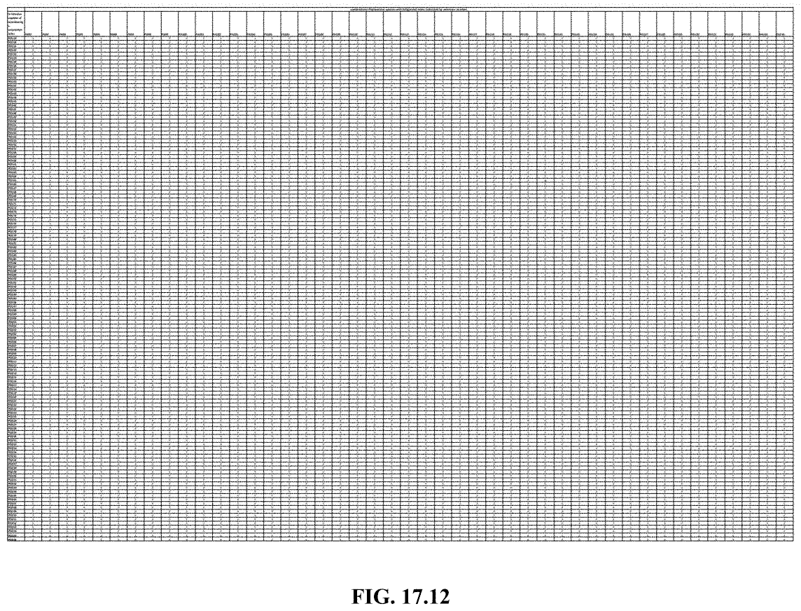

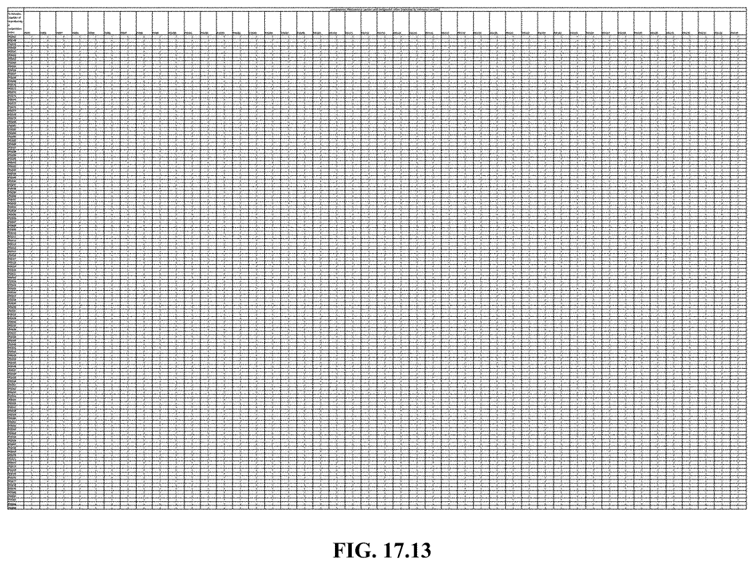

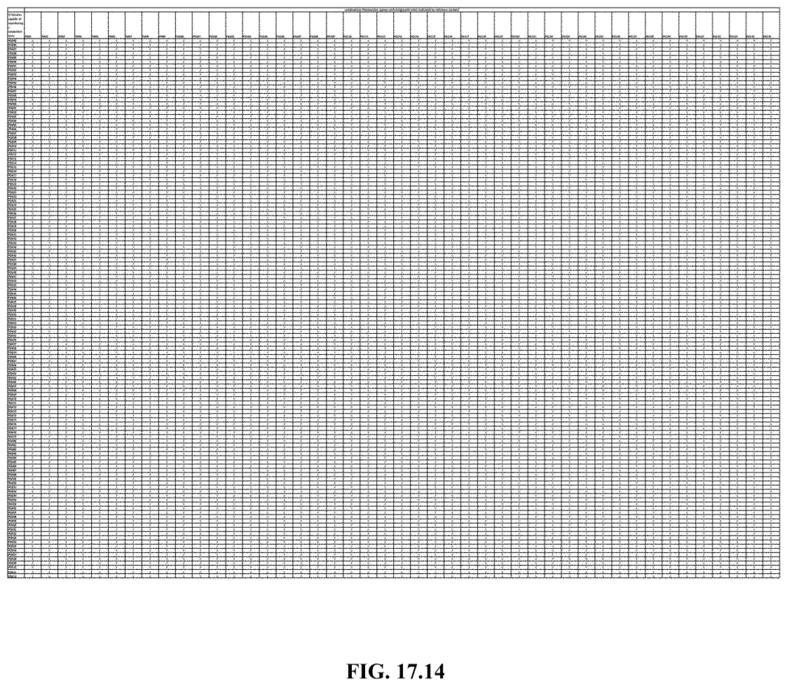

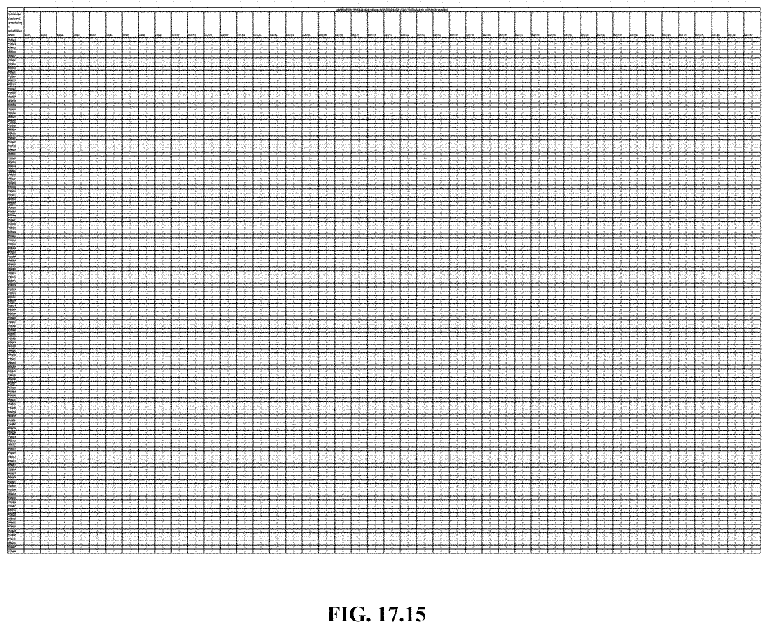

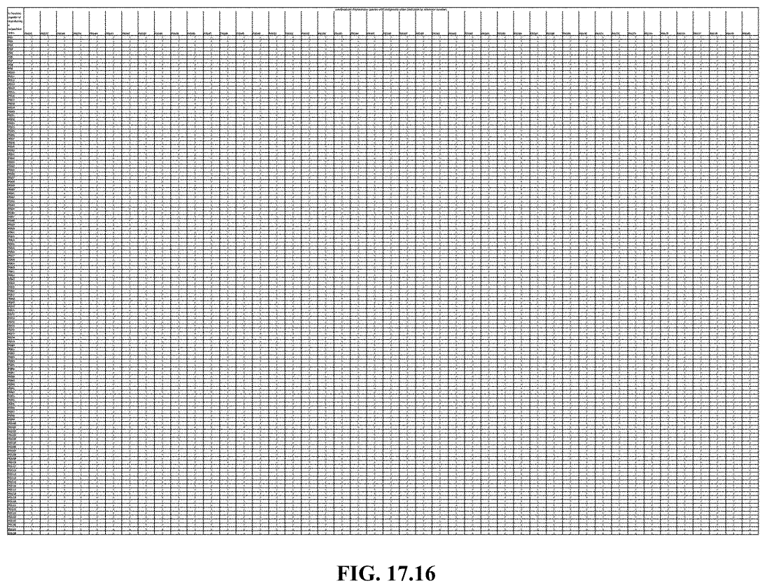

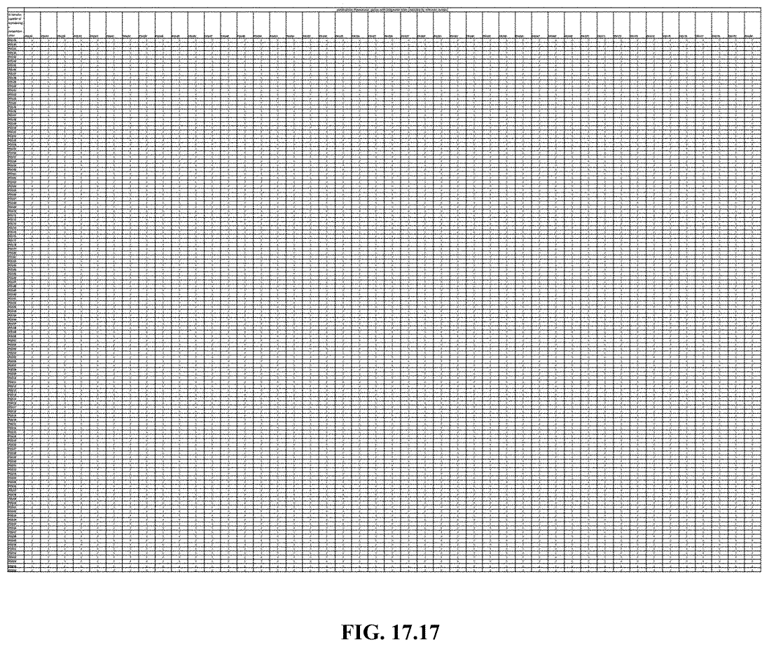

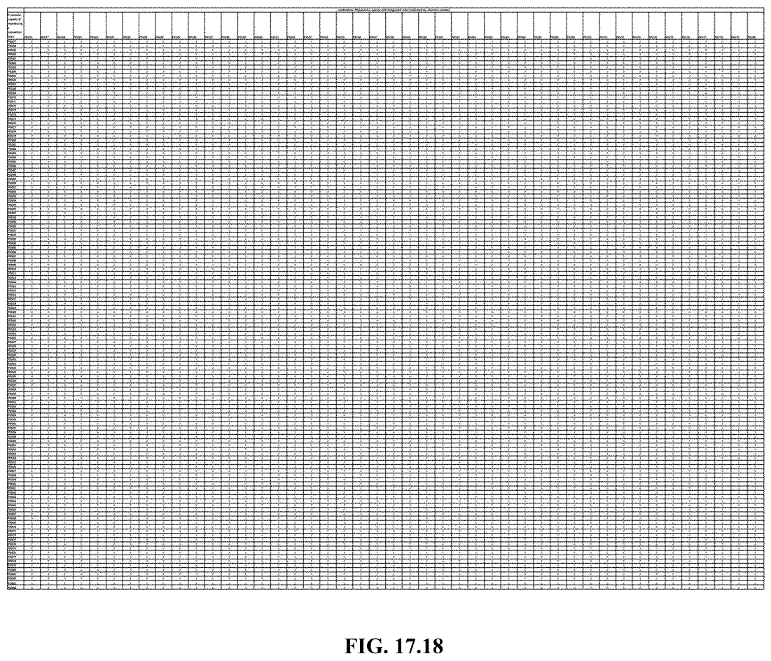

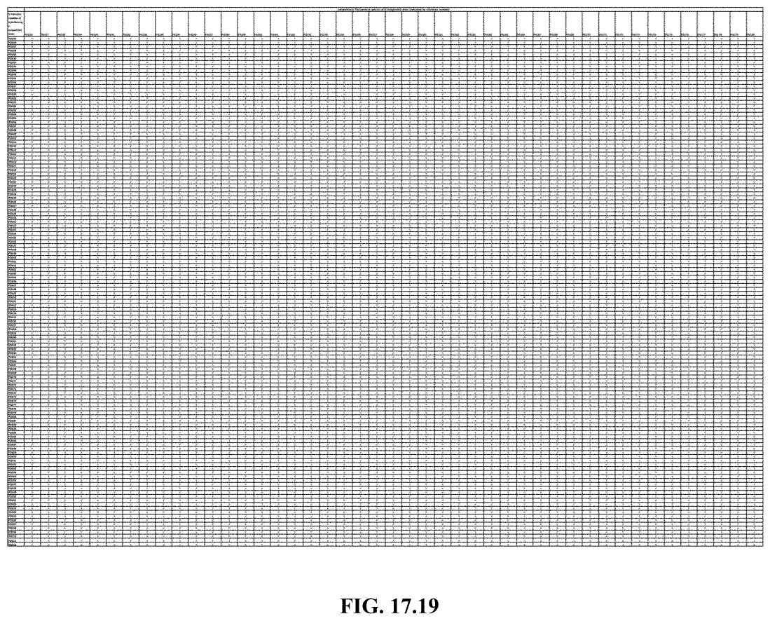

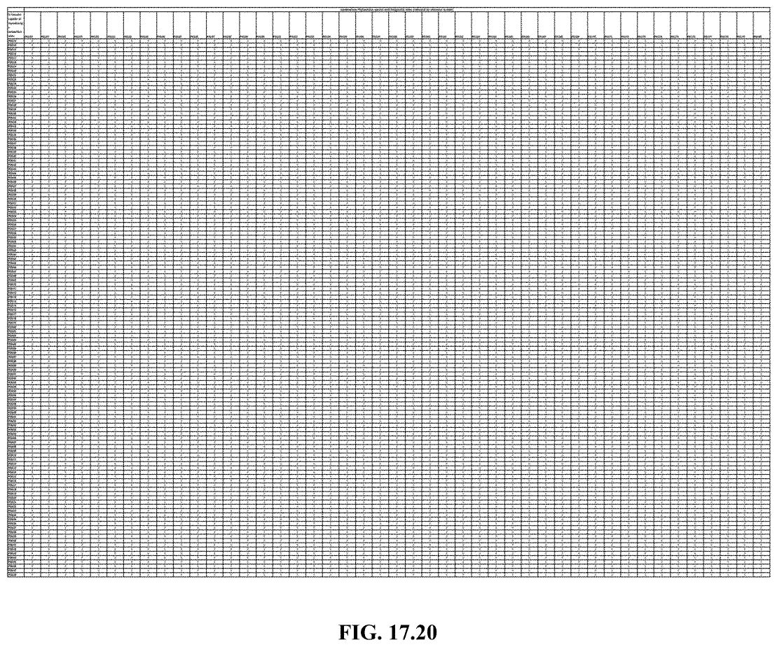

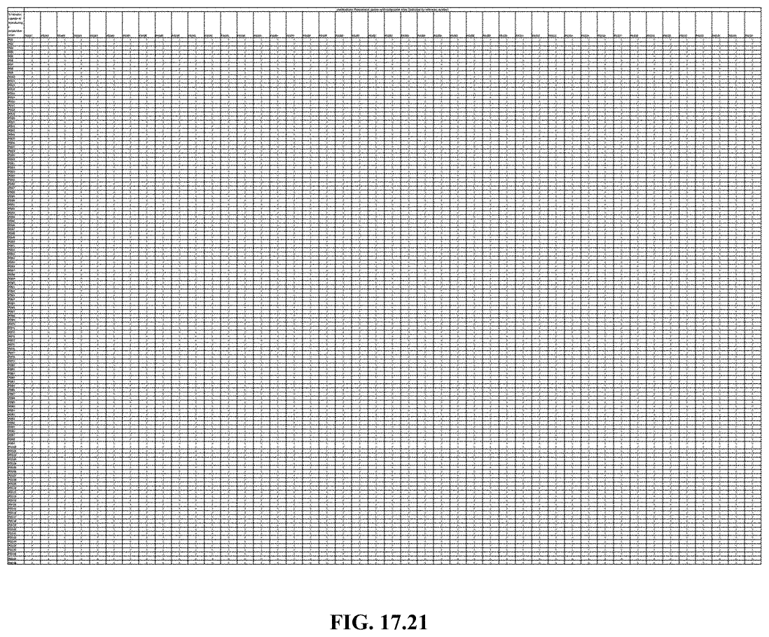

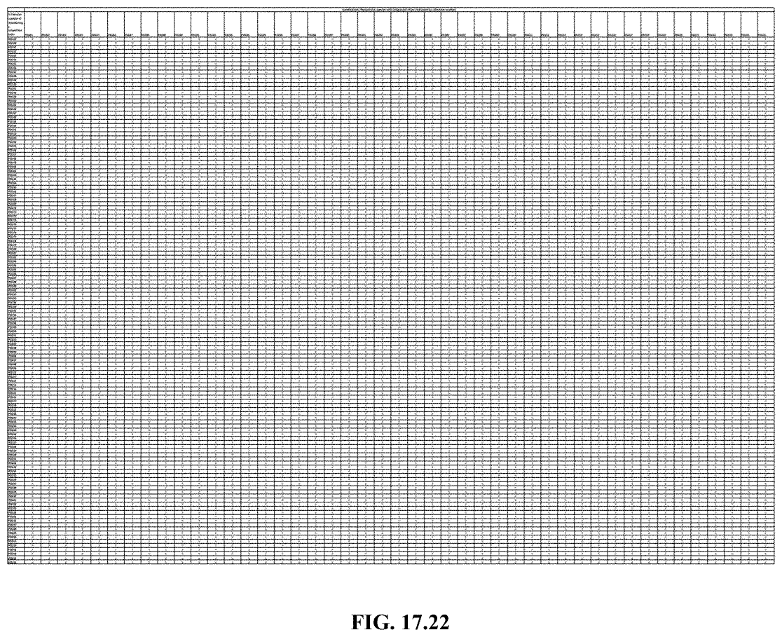

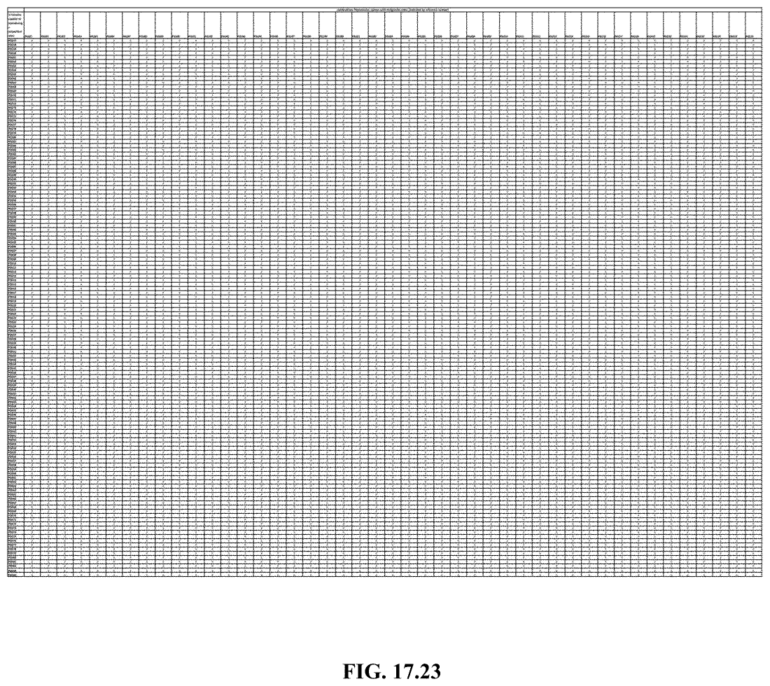

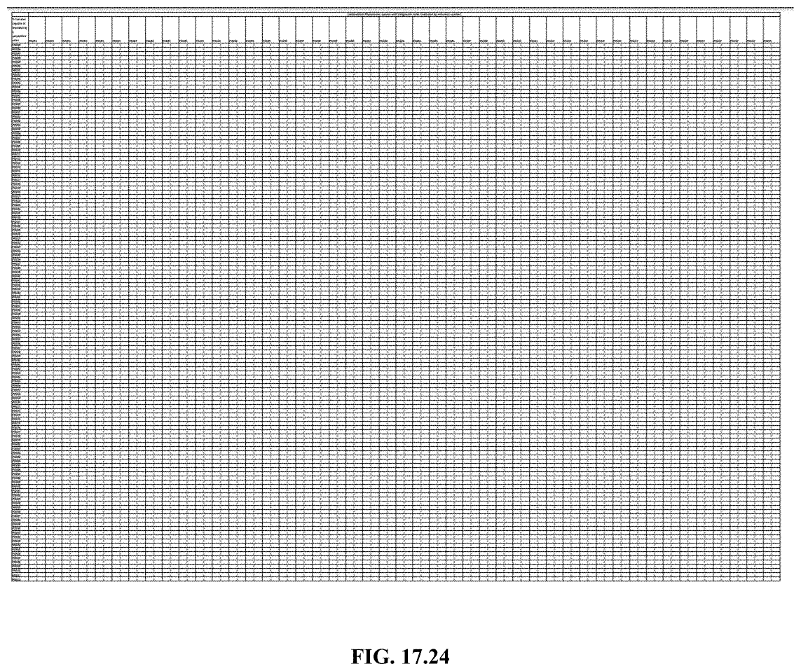

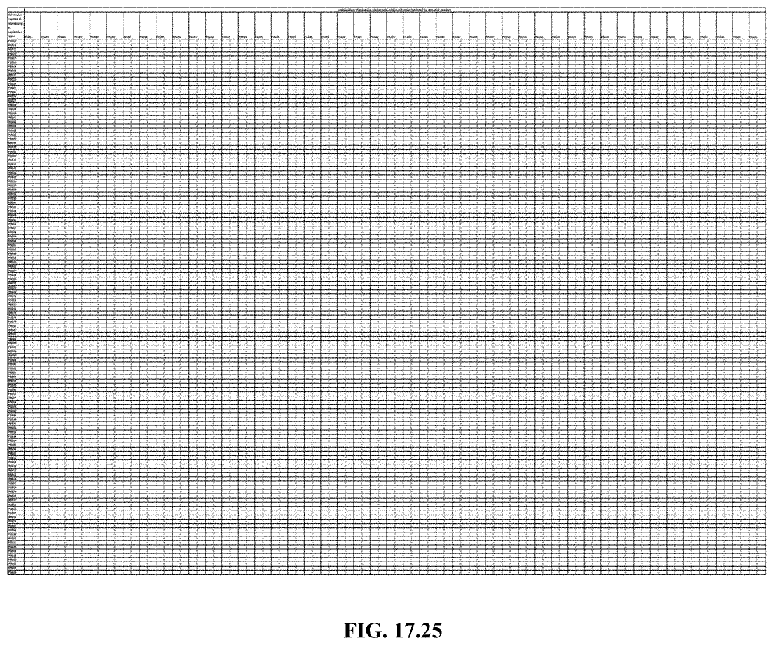

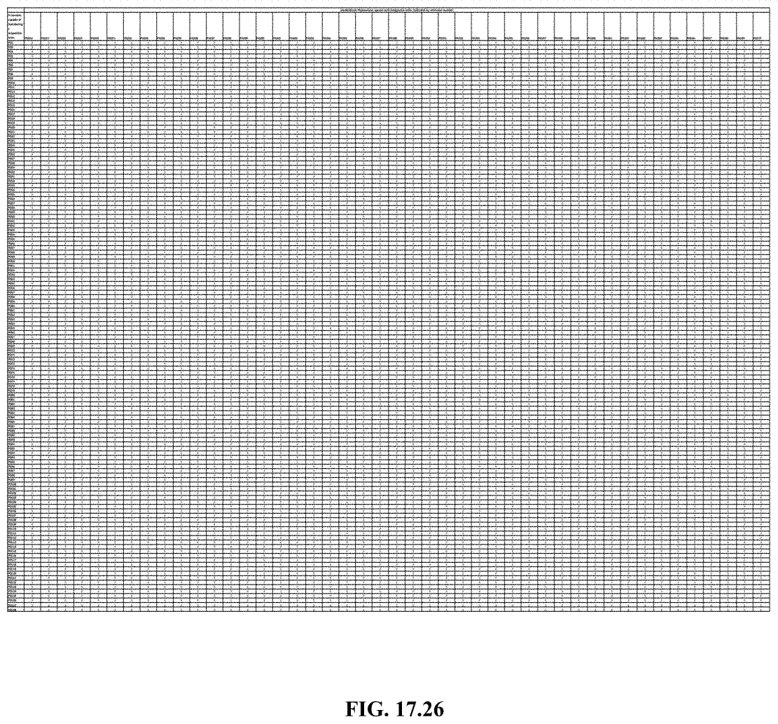

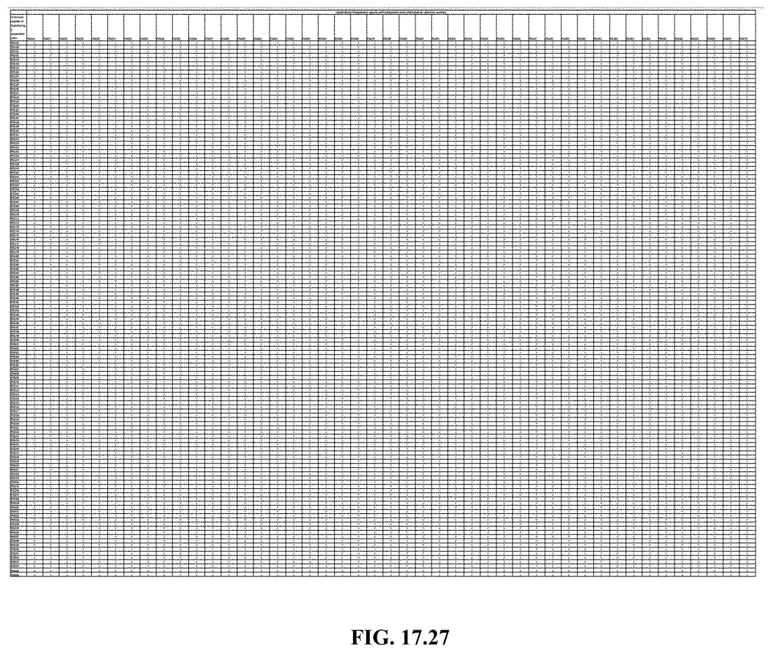

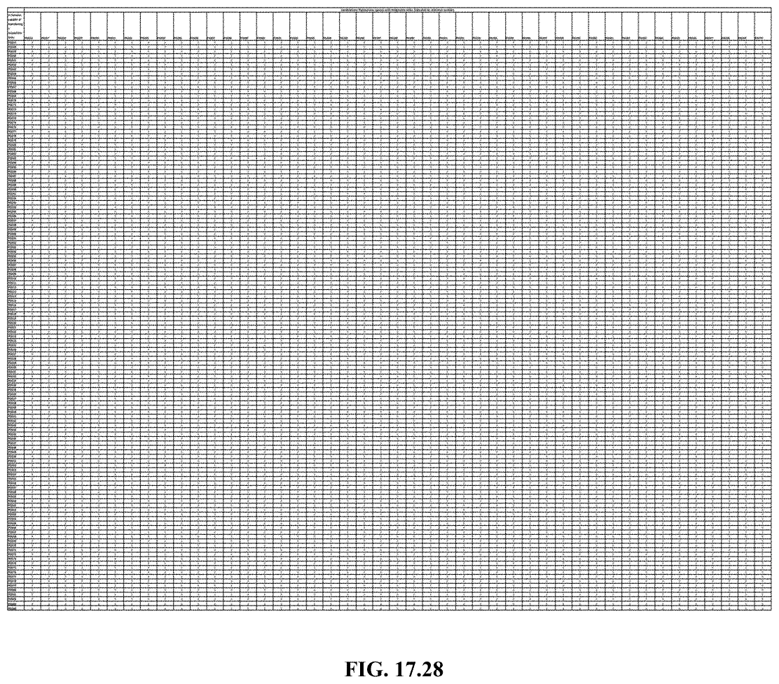

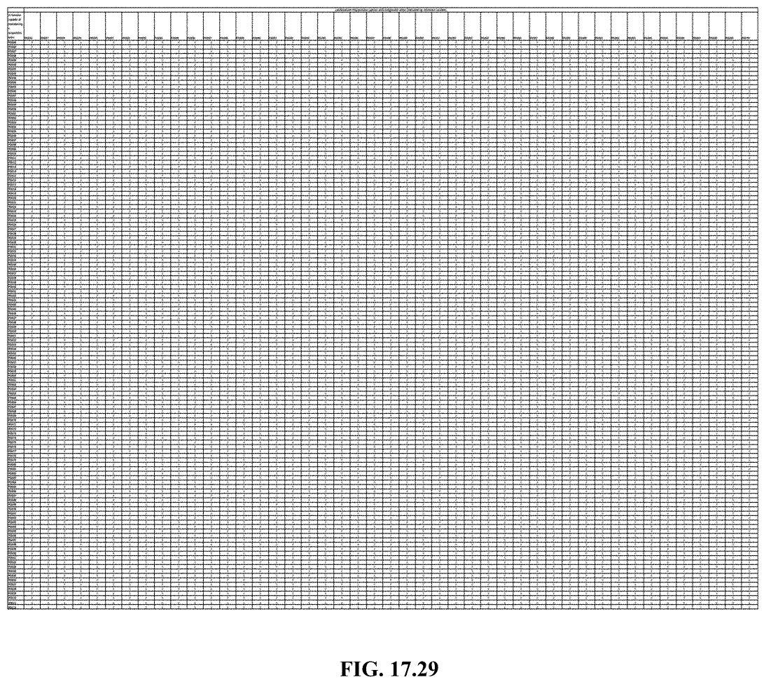

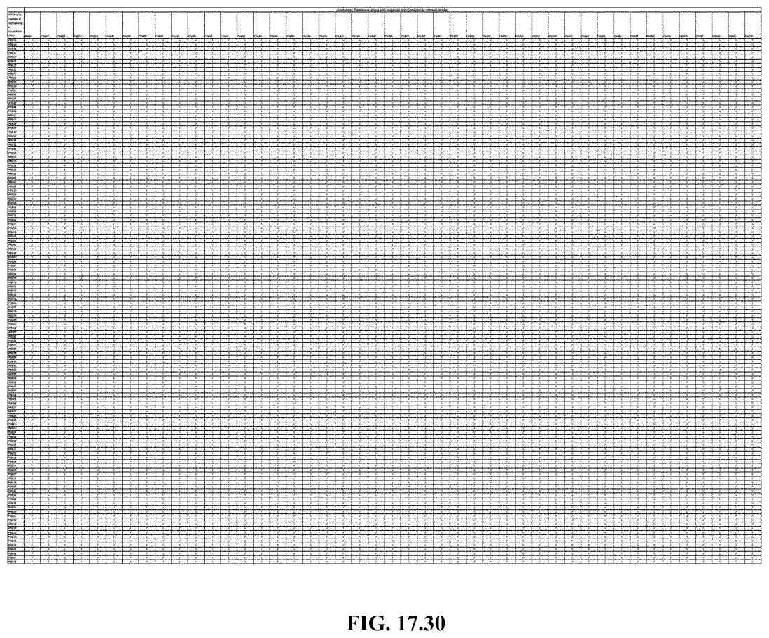

FIG. 17 presents further combinations of the combinations of the Phytoseiulus species x (groups of) Astigmatid mites (indicated by the PA1-PA270 reference numbers of FIG. 15) with the percentage of females capable of reproducing on the non-Tetranychid arthropod prey (P) x values for oviposition rate (indicated by the PO1-PO638 reference numbers of FIG. 13). Where “X” is presented, the specific combination is envisaged or applied to in embodiments of the various aspects of the present invention.

In order to understand the invention and to see how it may be implemented in practice, a plurality of preferred embodiments will now be described, by way of non-limiting example only, with reference to the following examples.

### EXAMPLE 1

Protocol for Rearing P. persimilis on non-tetranychid arthropod prey

In this example, rearing is done by feeding P. persimilis with a mixture comprising dead frozen developmental stages of C. lactis and sawdust or another carrier material (e.g. bran). The prey mites were immobilized by an immobilization treatment, e.g. by freezing them or by a gamma irradiation treatment, prior to using them as food.

Mites were fed with C. lactis immobilized by freezing, at the range of 10-1000 immobilized C. lactis prey individuals per one P. persimilis per day.

Exemplified growth conditions:

Temperature: in the range of 18° C.-30° C., particularly about 22° C.

Humidity: above 60%, particularly about 85%.

By using the aforementioned feeding regime, P. persimilis population was increased by an average of about 15%, per day.

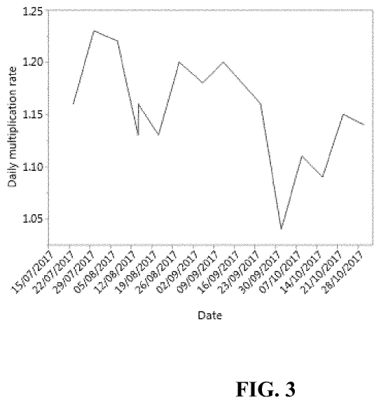

FIG. 3 graphically describes the daily multiplication rate of P. persimilis, feeding on a mixture of dead C. lactis eggs and mobile stages (killed by freezing) during a 14 weeks period. As can be seen, an average increase of between about 10% and about 20% in multiplication rate of P. persimilis was recorded per day. In other words, a λ of in the range of 1.05 to 1.23, in average a λ of 1.15, was measured during a 14 weeks period.

In a further experiment, the measuring period was 4 weeks, and the obtained λ value was 1.27.

Methods used for the above experiments:

A P. persimilis population was reared using dead C. lactis as prey at 22 degrees Celsius and 85% relative humidity in a mixture with sawdust. Every week the mixture was weighed, and four samples containing about 50 mg were taken, placed on a black adhesive tape and counted. Total population size was calculated according to these counts and 1500 individuals were left in the rearing each week. The multiplication rate was calculated by dividing the total number of the individuals found by 1500, giving the factor by which the population multiplied during this week. To switch to a daily multiplication rate, the 7th root of this number was taken according to the following formula:

$$λ = \text{?} \sqrt{\frac{N ( t}{N ( 0 )}}$$

$$\text{?} \text{indicates text missing or illegible when filed}$$

Where λ is the daily multiplication rate, N(O) is the number of mites left in the rearing in the former count (1500 in this case), N(t) is the number of mites found at the current count, and t=7.

### EXAMPLE 2

Rearing P. persimilis on different Astigmatid mite species

In this experiment, different mite species were tested as food for P. persimilis using the following protocol:

30 P. persimilis mites were isolated in modified Munger cells, and served with astigmatic mites, immobilized by freezing, of the species that are listed below. Food was replaced daily, and the mites were checked for feeding signs. The signs used as indicators were a full roundish body (contrary to a flat body of non-feeding mites), and whitish coloration in contrast to the usual orange color when feeding on spider mites.

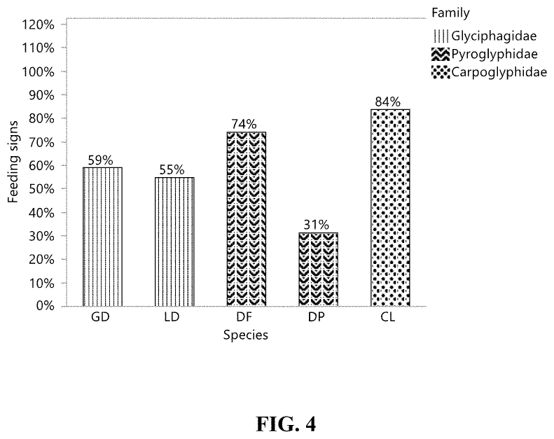

Reference is now made to FIG. 4 graphically presenting the percentage of P. persimilis showing feeding signs, as appeared by their body's shape and color, after given food for 3 consecutive days from each of the following prey species:

GD=Glyciphagus domesticus (Glycyphagidae family)

LD=Lepidogyphus destructor (Glycyphagidae family)

DF=Dermatophagoides farinae (Pyroglyphidae family)

DP=Dermatophagoides pteronisinus (Pyroglyphidae family)

CL=Carpoglyphus lactis (Carpoglyphidae family)

It can be seen that P. persimilis can feed on all of the above Astigmatic prey species, with varying efficiency.

### EXAMPLE 3

Reproduction of P. persimilis Predatory Mites on Dermatophagoides farinae (D. farinae) Prey

In this experiment, the prey used was D. farinae life stages immobilized by freezing. Mites were reared by the method as described in Examples 1 and 6. Rearing was maintained for 6 weeks, and the daily reproduction rate measured was about 1.05 on average. This demonstrates that P. persimilis can reproduce on D. farinae prey for more than two generations.

### EXAMPLE 4

Using Amblyseius swirskii as a prey for P. persimilis

In this experiment, 50 predatory mites were given immobilized (by freezing) mites of A. swirskii as food at 22 degrees Celsius, 85% RH, and checked daily. Mites were showing feeding signs by their large body shape and whitish coloration. When oviposition started, eggs were removed from the population, isolated, and hatchability was monitored. Hatching was noticed followed by maturation of the resulting larvae. When these mites matured, two were isolated to check for egg laying. These females did lay eggs, and hatching of the resulting eggs was observed. This demonstrates that P. persimilis can develop and reproduce on frozen A. swirskii as food for at least two generations, and that eggs laid at the third generation are viable.

### EXAMPLE 5

Juvenile Survival of P. persimilis Reared on the Non-Tetranychid arthropod Prey

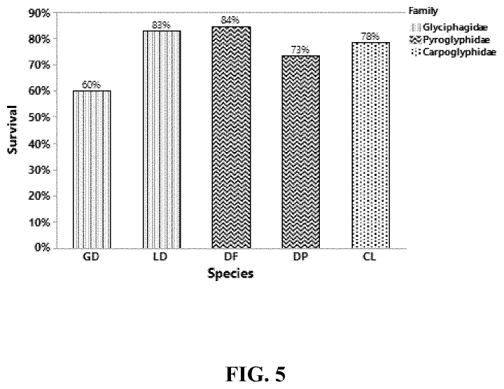

In this example, survival rate of P. persimilis was measured after 3 days of development, while feeding on non-Tetranychid arthropod prey. The methods used are as described in Example 2 above. As shown in FIG. 5, a juveninal survival rate of at least 60% and up to about 85% was observed when P. persinilis reproduced on non-Tetranychid arthropod prey, specifically on immobilized Astigmatid prey and more specifically on individuals of the family (i) Glyciphagidae, e.g. Glyciphagus domesticus (GD) and Lepidogyphus destructor (LD), (ii) Pyroglyphidae, e.g. Dermatophagoides farinae (DF) and Dermatophagoides pteronisinus (DP), and (iii) Carpoglyphidae, e.g. Carpoglyphus lactis (CL).

### EXAMPLE 6

Breeding and Selection for a P. persimilis Population with Increased Reproduction Rate on C. lactis as Prey

This experiment shows successful breeding and selection for a P. persimilis population adapted for rearing on C. lactis as prey. As shown in this example, the selected P. persimilis population is characterized by advantageous and desirable properties of significantly increased reproduction rate when reared on Astigmatid mite individuals.

Experimental Protocol:

Two different populations of P. persimilis were reared using C. lactis immobilized by freezing as prey, at 22 degrees Celsius and 85% relative humidity in a mixture with sawdust. The first population was a P. persimilis population bred and selected for adaptation for C. lactis as a factitious host prey (designated P+), the second population (designated P−) was the conventional or commercially available P. persimilis population available from BioBee Biological Systems Ltd. (Sde Eliyaho, Israel) and was reared upon its natural host, i.e. spider mites. This second population served as the reference/control population (i.e. not exposed to non-Tetranychid arthropod prey, such as C. lactis). Every week a mixture from each population was weighed, and four samples containing about 50 mg each were taken, placed on a black adhesive tape and counted. Total population size was calculated according to these counts and 1500 individuals were left for rearing each week. The multiplication rate was calculated by dividing the total number of the individuals found, by 1500, giving the factor by which the population multiplied during this week. To calculate the daily multiplication rate, the 7^{th} root of this calculated number was taken according to the following formula:

$$λ = \text{?} \sqrt{\frac{N ( t}{N ( 0 )}} ,$$

$$\text{?} \text{indicates text missing or illegible when filed}$$

where λ is the daily multiplication rate, N(O) is the initial number of mites left for rearing (i.e. 1500 mites), N(t) is the total number of mites found after rearing for a week period of time, and t=7.

It is noted that each population was maintained and measured for 4-10 weeks. The entire procedure was replicated for 3 times.

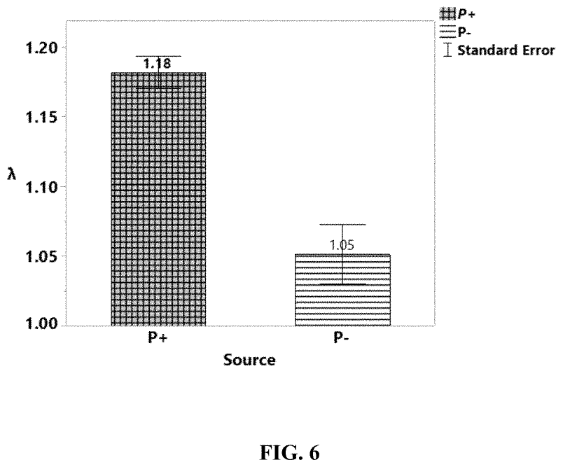

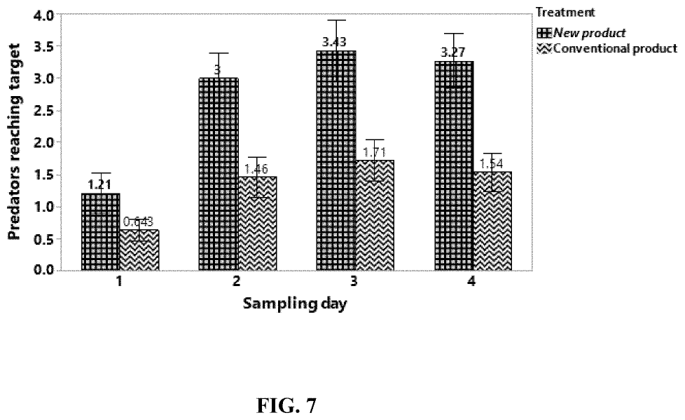

Reference is now made to FIG. 6 demonstrating the observed differences in the daily reproduction rate (represented by λ, the finite rate of increase) between the P. persimilis population bred and selected for adaptation for C. lactis as a factitious host prey (marked as P+ in FIG. 6), as compared to the conventional or commercially available P. persimilis population (reared upon its natural host, i.e. spider mites) used as a control (marked as P− in FIG. 6). The figure represents the means and the standard error found in the λ values during the trial.

As can be seen in FIG. 6, the P. persimilis population subjected to selection for improved adaptation to rearing upon C. lactis individuals (P+) demonstrated a significant increased daily reproduction rate of about 3.6 fold (P+/P−: 0.18/0.05) on C. lactis as a prey, as compared to the control P. persimilis population, not subjected to the breeding and selection process as inter alia described (P−).

Reference is now made to an embodiment of the present invention describing an exemplified scheme for obtaining P. persimilis populations selected for and/or adapted for reproductin on non-Tetranychid diet, such as Carpoglyphus lactis species.

I. From the P. persimilis population commercially available from BioBee Biological Systems Ltd. (Sde Eliyaho, Israel) (or a P. persimilis population reared on spider mites as was used up until the current invention, defined as P−), mites were subjected to feeding trials on Astigmatid mites and Amblyseius swirskii as prey, using the method described in Example 2. In these feeding trials, mites were subjected to various diets during 3 days, and tested for feeding signs and survival. The survivors of different feeding trials, on a variety of different prey types such as Astigmatid mites, were kept and new populations were formed from these survivors. These populations were further maintained on spider mites.

II. Additional different populations, collected from 18 distinct geographical locations, were obtained.These populations were also maintained on spider mites.

III. Samples of the different populations were pooled together to create one base population. This base population was also reared on spider mites.

IV. A sample from this base population was transferred to a new diet consisting of Carpoglyphus lactis individuals immobilized by freezing. Rearing of this base population continued on immobilized C. lactis for several generations (e.g. for about 1 year or more) and according to embodiments of the present invention it is herein defined as the selected (P+) population for reproduction on non-Tetranychid prey. Mites are fed with C. lactis life stages immobilized by freezing, at the range of 10-1000 C. lactis for one P. persimilis per day.

On the basis of the information provided in this application, it may be expected that this protocol can be extended also to other phytoseiulus species to render them capable to reproduce on (immobilized) non-terranychid arthropod prey, in particular (immobilized) Astigmatid mites, more in particular immobilized (non-hatching) eggs.

### EXAMPLE 7

Reference is now made to experimental results evaluating reproduction characteristics on non-tetranychid arthropod prey (i.e. Astigmatid species, particularly C. lactis immobilized individuals), of the P+ and P− P. persimilis populations, as described in Example 6.

P+ population, reproduced on immobilized non-tetranychid arthropod prey (e.g. C. lactis eggs and mobile stages, both immobilized by freezing) as herein described by Examples 6.

P− population, a commercially available population and/or any P. persimilis population reproduced using spider mites, as was used up until the current invention (not exposed to a non-tetranychid arthropod prey).

The reproduction parameters tested included: daily oviposition rate, female survival (%) and percentage of females that lay eggs.

To compare the above reproduction parameters of P. persimilis population fed with immobilized (e.g. by freezing) C. lactis between the P+ and the P− populations, the following test was carried out.

Gravid females were tested in individual cells in which dead C. lactis eggs and larvae (immobilized by freezing) were supplied. These females were taken from either the P+ population reared on immobilized C. lactis, or the P− population reared on spider mites. To assure that all females were well fed prior to the trial, gravid females were chosen directly from both rearing populations (without a starvation phase that is usually applied to by the current invention, when harvesting P. persimilis from spider mites rearing and altering its diet). From each treatment 20 replicates were performed.

The cells were incubated at 22 degrees Celsius, and relative humidity of 85%, and checked every two or three days during one week. On each check mite's survival, color and the number of eggs laid were recorded. Food was replenished on each check. The sum of eggs deposited during the test period per female was divided by 7 to obtain the daily oviposition rate. Results are summarized in the following Table 1:

TABLE 1
Comparison between P+ and P− populations
in reproduction parameters

| | | | Percentage | Percentage | Eggs/ | Eggs/ |
|---|---|---|---|---|---|---|
| | Survival | Survival | ovipositing | ovipositing | day | day |
| Days | P− | P+ | P− | P+ | P− | P+ |
| 2 | 86% | 100% | 63% | 90% | 0.64 | 1.43 |
| 4 | 56% | 100% | 0% | 94% | 0 | 1.63 |
| 7 | 25% | 80% | 25% | 94% | 0.07 | 1.47 |

The results presented in Table 1 clearly show that there are significant differences between the P+ and the P− populations on the level of oviposition rate, female survival, and percentage of females that lay eggs. In all tested parameters, higher values were observed and recorded for the P. persimilis population that was reared and reproduced on immobilized C. lactis developmental stages (P+). These values include female survival percentage in the range of 80% to 100% (an average value of 93.3%); percentage of females that lay eggs in the range of 90% to 94% (an average value of 92.6%); and daily oviposition rate in the range of 1.43 to 1.63 (an average value of 1.51 eggs/day/female). This is in comparison to significantly lower values recorded for the P− population, namely, female survival percentage in the range of 25% to 86% (an average value of 55.6%); percentage of female that lay eggs in the range of 25% to 63% (an average value of 29.3%); and daily oviposition rate in the range of 0.07 to 0.64 9an average value of 0.23 eggs/day/female).

It should be noted that at the first test day, performed two days after the setup of the trial, some differences already appeared between the mites coming from the different populations. Survival, daily oviposition, and percentage of the mites that laid eggs were significantly higher for mites coming from the P+ population. While some P− mites did lay some eggs on this day, the eggs appeared orange, indicating that they were metabolized from nutrients the mites acquired while being reared on spider mites before this diet was replaced by immobilized stages of C. lactis diet.

On the second test day, performed at day 4, the differences were dramatically increased. While none of the mites from the P+ population died, survival was only 56% for the P− population. In addition, all of the mites but one from the P+population laid eggs, while none did from the P− population and the daily oviposition rate increased for the P+ population, and was zero for the P− population. This tendency was maintained for all parameters checked on the third test day (day 7 of the trail), meaning, significantly improved reproduction values for the P+ population over the P− control population.

The results presented in Table 1 demonstrate that there are significant and dramatic differences between the P+ and the P− populations on the level of oviposition rate, female survival, and percentage of females that lay eggs. The Phytoseiulus predatory individuals of the population subjected to the rearing method of the present invention present markedly enhanced capability to reproduce on the non-Tetranychid arthropod prey as compared to currently available populations of the same Phytoseiulus species reared on spider mites.

### EXAMPLE 8

A Process for Rearing P. persimilis using non-Tetranychid arthropod Immobilized Prey

Reference is now made to an exemplified method used for processing non Tetranychid arthropod immobilized prey (e.g. Carpoglyphus lactis eggs) to be served as a food for P. persimilis predatory mites or other Phytoseiulus predatory species. This example is an embodiment within the herein disclosed system and method for rearing mite species of the genus Phytoseiulus using Astigmatid mites as a prey. In this embodiment, Carpoglyphus lactis eggs are separated from the mite population by sieving. Then, while still wet, they are mixed with sawdust and water, in a manner that coats the sawdust particles with a thin layer of eggs. After this process the mixture is freezed. This mixture is served to P. persimilis as food. This process allows improved accessibility of the predators to the eggs, and additionally improves the efficiency of the predatory mite rearing process.

### EXAMPLE 9

Development and Reproduction of P. persimilis Populations from Distinct Geographic Sources on Non-Tetranychid Arthropod Prey

This example is aimed at testing the capability of completion of the life cycle on non-Tetranychid arthropod prey, by P. persimilis populations derived from distinct sources or distinct geographical origins or locations. Towards this end, P. persimilis populations originating from 3 distinct geographical locations (e.g. more then 1000 km in distance from each other) were tested on C. lactis (immobilized by freezing) as a prey. From each population, a cohort of about 50 P. persimilis eggs was placed in study chambers together with immobilized C. lactis eggs and juveniles. These populations were maintained at 22 degrees Celsius, and RH of 85%. After one week, the populations were observed. It was revealed that in all of the populations tested, a fraction of the mites developed, changed colour to beague-white, and laid eggs. This demonstrates that P. persimilis populations from distinct geographical origins are capable of developing and reproducing on non-Tetranychid arthropod prey, for example C. lactis.

To see whether a subsequent generation (e.g. a second generation) of the above tested populations would be able to complete its life cycle, eggs laid by the first generation of one of the populations, were moved to a new chamber and fed with the same diet. These eggs hatched, developed to adults that copulated, and laid eggs. This demonstrates that P. persimilis populations originating from distinct sources (e.g. distinct geographical locations) and subjected to the rearing method of the present invention are able to reproduce on an alternative diet (i.e. non-Tetranychid arthropod prey such as Astigmatid prey) for more than one generation.

### EXAMPLE 10

Phytoseiulus longipes Reared on C. lactis as Prey

Reference is now made to an example in which rearing is done by feeding Phytoseiulus longipes (P. longipes), as a further representative example of the Phytoseiulus genus, with C. lactis life stages (immobilized by freezing) as a prey.

Exemplified rearing protocol: A P. longipes population was reared using immobilized C. lactis as prey at 22 degrees Celsius and 85% relative humidity in a mixture with sawdust, using the rearing methods as described in Examples 1 and 6. Mites showed feeding signs by changing their color from typical redish to white, as shown above for P. persimilis fed on C. lactis (see FIGS. 1 and 2). In addition, all of the different life stages of the P. longipes mites have been observed, indicating that this species completed its development cycle on this alternative diet. The rearing was maintained for three weeks, showing that the P. longipes population can be reared upon immobilized C. lactis diet for at least this period of time.

When rearing was maintained for 6 weeks, the daily reproduction rate measured was 1.08 on average. This demonstrates that P. longipes can also reproduce on immomilized C. lactis prey for more than two generations.

### EXAMPLE 11

Predatory Behavior Towards a Tetranychidae

To assess the predatory behavior of P. persimilis reared using non-Tetranychid arthropod prey (e.g. Astigmatid species), towards spider mites, several trials were conducted.

A. Preying and Oviposition on Leaf Discs

Thirty adult females derived from P. persimilis population selected for reproducing on non-Tetranychid alternative food source, particularly on Astigmatid prey, were placed individually on spider mite infested leaf discs. The method used was according to the IOBC protocol for testing fecundity of P. persimilis (van Lenteren J C, ed. 2003. Quality Control and Production of Biological Control Agents: Theory and Testing Procedures. Wallingford, UK: CABI Publ. 327 pp.) . Four different tests were carried during one year.

It was observed that P. persimilis mites selected and reproduced by the method of the present invention, and placed on spider mite infested leaf discs, changed their beague-white colour back to red-orange within a few hours. The average fecundity score (e.g. daily reproduction rate) of these tests was 19.85 eggs per female per 5 days period (3.97 eggs/female/day).

It is emphasized that this result is significantly above the official accepted threshold of 10 eggs per female per 5 days (see van Lenteren J C, ed. 2003. Quality Control and Production of Biological Control Agents: Theory and Testing Procedures. Wallingford, UK: CABI Publ. 327 pp). These results demonstrate that predatory mites reared using the new and highly desirable rearing system of using non-Tetranychids as alternative food source for P. persimilis, maintained and even increased their ability to consume spider mites and reproduce on spider mite prey.

b. Ability to Locate Tetranychid Prey

To test whether the new P. persimilis population mites selected for reproduction on non-Tetranychid prey (e.g. C. lactis), maintain their ability to locate spider mite patches on the plant, the following trials were conducted:

Cucumber plants were infested with 25 spider mites on their top leaf. Three days later, 10 P. persimilis females were released on the bottom leaf of the plant. These females originated from two different treatments — either the conventional P. persimilis rearing on spider mites (referred to as conventional product in FIG. 7), or mites reared on dead C. lactis as prey (referred to as new product in FIG. 7). Thirteen plants (replicates) were tested for each treatment. To assess the ability to locate the prey patch and reach it, the top leaf was observed daily along three days, and the number of P. persimilis reaching this leaf were recorded.

The results shown in FIG. 7 present the number of predators that were found on the infested leaf at each treatment each day. Bars indicate Means +-standard error.

It can be seen that the predatory mites reared using the non-Tetranychid alternative as prey, reached the spider mites prey patches at significantly higher rates than the conventionally reared predatory mites (predatory mites reared on spider mites). This indicates that not only the prey location ability was not negatively affected by the new rearing technology, but in fact, it was significantly improved by about 1.5-3 fold, e.g. by about 2 fold, relative to P. persimilis conventionally reared on spider mites (referred to as commercial product in FIG. 7).

c. Ability to Control a Spider Mite Population

To test whether the new P. persimilis population of the present invention maintains its ability to control spider mite patches on the plant, the following trial was conducted:

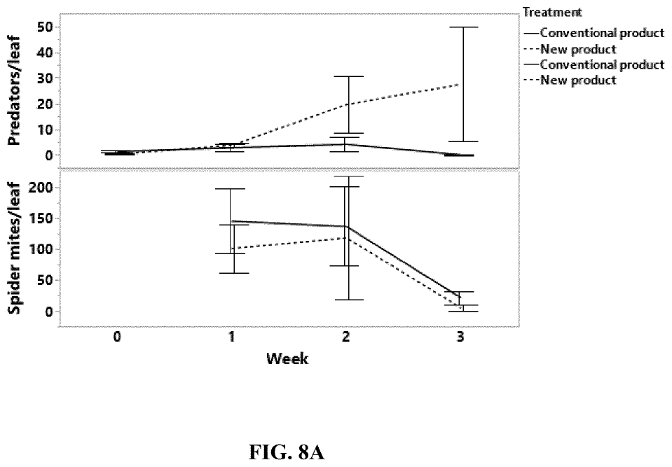

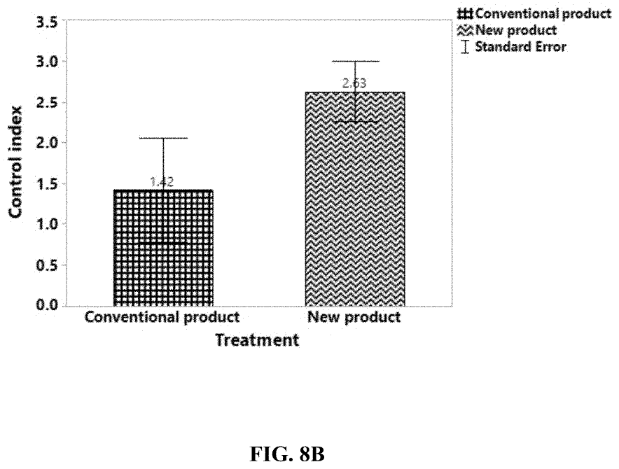

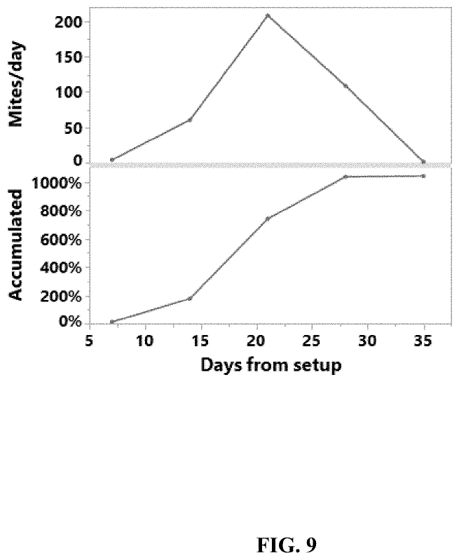

Groups of four cucumber plants, about one meter high, were placed in cages, touching each other. The top leaf of one plant found in one of the sides of the group, was infested with 35 spider mites. Two days later, 20 P. persimilis females were introduced on the bottom leaf of the most distant plant from the infested one. These females originated from two different treatments—either the P. persimilis conventionally reared on spider mites (referred to as conventional product in FIG. 8), or P. persimilis mite population of the present invention reared on dead C. lactis as prey (referred to as new product in FIG. 8). Each treatment was replicated 8 times (8 cages per treatment). Spider mites and P. persimilis were counted weekly on the infested leaf In addition to the counts, three weeks after the introduction of the predators, the cages were monitored by a scout unaware to the identity of the treatments, and scored according to the level of control of the spider mites in each cage. The following index was used to score the ability of P. persimilis to control spider mites:

0—Spider mites spreading out with no control

1—Spider mites spreading out, but P. persimilis gains control

2—Spider mites under control

3—Full control of spider mites obtained

Reference is now made to FIG. 8A graphically showing the number of predators/leaf and spider mites/leaf found in each sampling week under the different treatments.

Reference is now made to FIG. 8B graphically showing the spider mites control index found three weeks after predator's introduction at each treatment.

The results depicted in FIGS. 8A and 8B demonstrate that the plant that were treated with the new P. persimilis population reared on non Tetranychid prey, showed higher numbers of predatory mites, lower number of spider mites, and a higher control index. This indicates that the prey control ability of the new P. persimilis population reared on non- Tetranychid arthropod prey, was not negatively affected, and even surprisingly was improved by about 2 fold relative to the conventionally-reared, P. persimilis population.

### EXAMPLE 12

Slow release system for P. persimilis

Reference is now made to a description of a controlled release system or device for P. persimilis according to some embodiments of the present invention. Mixture containing about 120 mobile stages and 80 eggs of P. persimilis mites reared on C. lactis immobilized prey (e.g. dead prey) and additional prey and sawdust as a carrier, was inserted into four paper sachets, commonly used for slow release of predatory mites other than P. persimilis. The sachets were placed on an adhesive tape or surface under controlled conditions (22 degrees Celsius and 85% humidity). The adhesive tape was replaced once a week, and P. persimilis mites which appeared on it were counted to assess the release rate from the container.

Reference is now made to FIG. 9 graphically illustrating mites release rate from the sachets as a function of the number of days since the setup of the experiment.

In this figure, the X axis stands from the number of days since the setup of the trial, the top Y axis represents the number of mites/day released from the group of four sachets, and the bottom Y axis represents the accumulated number of mites compared to the initial number put in the sachets.

As can be seen in FIG. 9, mites are continuously released from the container for a period of 35 days, with a release peak occurring around day 21 (between days 14 and 21). The amount of mites leaving the sachets in total reaches about 10 times the initial amount of mites (mobile stages + eggs) put into the sachet in the starting point of the experiment. The predatory mite release rate was up to 200 mites/day from four sachets. This example demonstrates that a slow or controlled release system for P. persimilis (for at least about 20 days) is constructed, based on the rearing composition and method of the present invention.

### EXAMPLE 13

Slow Release of the Mites in the Field

This example shows the performance of a slow release system of the current invention (e.g. as described in Example 4 above) under greenhouse conditions.

Sweet pepper plants were planted in the greenhouse, and exposed to three different treatments in 5 replicates:

a) A slow release sachet containing 30 P. persimilis individuals was applied to the plants 13 days before the plants were infested with spider mites.

b) A slow release sachet containing 30 P. persimilis individuals was applied to the plants 6 days before the plants were infested with spider mites.

c) Control plants which were not exposed to P. persimilis.

The sachet was located on the lower parts of 1 meter tall plants. Infestation was carried out by stapling a spider mite infested bean leaf to one of the top leaves of the plant. The mite population on each plant was sampled 3 days after the plants were infested. The spider mites and P. persimilis mites found on the infested leaf or above it were recorded.

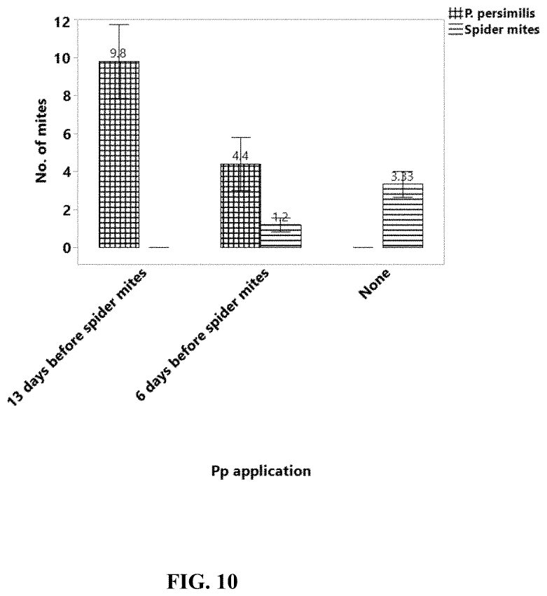

Reference is now made to FIG. 10 graphically illustrating P. persimilis (Pp) and spider mite counts of plants exposed to the slow release system of the present invention as compared to control plants. As can be seen, the predatory mites were found on plants exposed to both of the P. persimilis treatments. Furthermore, the amounts of spider mites in the P. persimilis treated plants were rapidly reduced compared to the control plants. More particularly, an inverse correlation was observed between the P. persimilis counts and the spider mites counts, namely, the more P. persimilis mites were found on the plants, the less spider mites were counted. This experiment clearly demonstrates that P. persimilis mites, and more specifically, the composition of the present invention, is effective against spider mite infestation. The P. persimilis slow release system of the present invention reduced the spider mites population on the plant, despite the relatively long time (about 6 to 13 days) elapsing between the P. persimilis application and the spider mites arrival to the plant. This shows the effectiveness of the P. persimilis composition and slow release system as herein described in controlling spider mite infestations.

### EXAMPLE 14

Oviposition of P. persimilis on a Non-Tetranychid Arthropod that is not a Mite

In this trial, gravid females were taken from the rearing on immobilized C. lactis life stages (dead C. lactis individuals), isolated and given a diet of decapsulated Artemia cysts at 22 degrees Celsius and 100% RH. It was observed that all of the mites readily fed and changed color when given Artemia within a day. Eggs were laid by the P. persimilis mites, indicating they can reproduce on Artemia cysts. As a skilled person will understand, further optimization and adjustments to the results may be made (which are within the ambit of the skill person).

This example shows that Phytoseiulus mite species are capable of reproducing on a non-tetranychid arthropod prey using the method of the invention, as inter-alia disclosed. The present disclosure demonstrates the achievement of oviposition of Phytoseiulus mites on non-phytophagous mites, such as Astigmatid mites as well as on non-tetranychid arthropod prey that is not a mite, such as Artemia, using the methods of the invention.

To conclude, the present invention provides for the first time a P. persimilis population characterized by increased reproduction rate trait reared upon an alternative diet of non-Tetranychid arthropod prey, preferably Astigmatid mites such as C. lactis individuals as a prey. This enables a highly desirable, revolutionary, indoor production of improved P. persimilis predatory mites exhibiting increased yield when reared upon Astigmata species, as compred to currently available P. persimilis mites, which demonstrate significantly reduced reproduction rate and yield when reared upon the same Astigmata species prey.

### REFERENCES

Chant, D. A. & McMurtry, J. A. (2006). A review of the subfamily Amblyseiinae Muma (Acari: Phytoseiidae): part VIII. The tribes Macroseiini Chant, Denmark and Baker, Phytoseiulini n. tribe, Africoseiulini n. tribe and Indoseiulini Ehara and Amano. International Journal of Acarology 32, 13-25.

Simmonds, S. P. (1970). The Possible Control of Steneotarsonemus pallidus on Strawberries by Phytoseiulus persimilis. Plant pathology 19, 106-107.

McMurtry, J. A. & Croft, B. A. (1997). Life-styles of phytoseiid mites and their roles in biological control. Annual Review of Entomology, 42, 291-321.

Helle, W. & Sabelis, M. W. (1985). Spider Mites. Their Biology, Natural Enemies and Control, Vol. 1B. Elsevier, Amstedam.

Gerson, U., Smiley, R. L. & Ochoa, R. (2003). Mites (Acari) for Pest Control; Blackwell Science Ltd.: Oxford, UK.

Walzer, A. & Schausberger, P. (1999). Cannibalism and interspecific predation in the phytoseiid mites Phytoseiulus persimilis and Neoseiulus californicus: predation rates and effects on reproduction and juvenile development BioControl 43: 457-468.

Yao, D. S. & Chant, D. A. (1989). Population growth and predation interference between two species of predatory phytoseiid mites (Acarina: Phytoseiidae) in interactive systems. Oecologia 80: 443-455.

Walzer, A., Paulus, W. & Schausberger, P. (2004) Ontogenetic shifts in intraguild predation on thrips by phytoseiid mites: the relevance of body size and diet specialization. Bulletin of Entomological Research, 94, 577-584.

van de Vrie, M., McMurtry J. A. & Huffaker C. B. (1972) Ecology of tetranychid mites and their natural enemies: A review: III. Biology, ecology, and pest status, and host-plant relations of tetranychids. Hilgardia 41(13):343-432.

1-57. (canceled)

58. Predatory mite population comprising Phytoseiulus predatory individuals,
i) wherein at least 10% of female individuals of the population is capable of reproduction on a non-tetranychid arthropod. prey, preferably on an immobilized non-tetranychid arthropod prey, such as on a non-phytophagous prey, preferably on an. Astigmatid prey, most preferably on an immobilized Astigmatid prey, such as an immobilized Astigmatid prey, in particular a Carpoglyhus prey, having immobilized. life stages comprising immobilized eggs,
ii) wherein the population is characterized. by a daily oviposition rate of at least 0.55 eggs/day/female, such as >0.60, >0.65, >0.70, >0.75, >0.80, >0.90, >0.95, >1.00, >1.05, >1.10, >1.15, >1.20, >1.25, >1.30, >1.35, >1.40, >1.45, >1.50, >1.55, >1.60, >1.65, >1.70, >1.75, >1.80, >1.85, >1.90, >1.95, or >2.00 eggs/day/female, while preying on non-tetranychid arthropod prey, preferably on an immobilized non-tetranychid arthropod prey, such as on a non-phytophagous prey, preferably on an Astigmatid prey, most preferably on an immobilized Astiqmatid prey, such as an immobilized Astigmatid prey having immobilized life stages comprising immobilized eggs, or
iii) wherein the population is characterized by improved reproduction on non-tetranychid arthropod prey, preferably on an immobilized non-tetranychid arthropod prey, such as on a non-phytophagous prey, preferably on an. Astigmatid prey, most preferably on an immobilized. Astigmatid prey, such as an immobilized Astigmatid prey having immobilized life stages comprising immobilized eggs, as compared to a control Phytoseiulus predatory population of the same species, reared on Tetranychid prey as the sole food source,
wherein preferably predatory individuals are from a species selected from Phytoseiulus fragariae, Phytoseiulus longipes, Phytoseiulus macropilis, Phytoseiulus persimilis and Phytoseiulus robertsi.

59. Predatory mite population according to claim 53(1),
wherein at least 15%, at least 20%, at least 25%, at least 30%, at least 35%, at least 40%, at least 45%, at least 50%, at least 55%, at least 60%, at least 65%, at least 70%, at least 75%, at least 30%, at least 85%, at least 90%, at least 95%, or at least 99% of female individuals of the population are capable of reproducing on a non-tetranychid arthropod prey, preferably on an immobilized non-tetranychid arthropod prey, such as on a non-phytophagbus prey, preferably on an Astigmatid prey, most preferably on an immobilized Astigmatid prey, such as an immobilized Astigmatid prey having immobilized life stages comprising immobilized eggs,
wherein the at least 10% of female individuals of the population are capable of oviposition on the non-Tetranychid arthropod prey, preferably on immobilized Astigmatid prey having immobilized life stages comprising immobilized eggs,
wherein the population has a daily oviposition rate of at least 0.50, such as >0.60, >0.65, >0.70, >0.75, >0.30, >0.90, >0.95, >1.00, >1.05, >1.10, >1.15, >1.20, >1.25, >1.30, >1.35, >1.40, >1.45, >1.50, >1.55, >1.60, >1.65, >1.70, 1.75, >1.30, >1.85, >1.90, >1.95 or at least 2.00 eggs/day/female on the non-Tetranychid arthropod prey, preferably on immobilized Astigmatid prey having, immobilized life stages comprising immobilized eggs,
wherein the population has a daily oviposition rate of at least 0.55, such as >0.60, >0.65, >0.70, >0.75, >0.80, >0.90, >0.95, >1.00, >1.05, >1.10, >1.15, >1.20, >1.25, >1.30, >1.35, >1.40, >1.45, >1.50, >1.55, >1.60, >1.65, >1.70, >1.75, >1.80, >1.85, >1.90, >1.95, or >2.00 eggs/day/female, when using the non-tetranychid arthropod preyas the sole food source,
wherein the at least 10% of female individuals are capable of completing a full ontogenetic cycle, when using the non-tetranychid arthropod prey as the sole food source,
wherein the population is characterized by a juvenile and/or female survival rate of at least 40%, preferably at least 45%, 50%, 60%, 65%, 70%, 75%, 80%, 85%, 90%, or at least 95% on the non-Tetranychid prey,
wherein the at least 10% of female individuals of the population are characterized by capability to produce female offspring in a number of subsequent generations, wherein. the number of subsequent generations is at least 1, such as at least 2, such as in at least 3, 4, 5, 6, 7, 8, 9 at least 10 generations,
wherein the population is characterized by a daily reproduction rate in the range of about 1.10 -1.40, such as 1.15-1.40, 1.20-1.40, 1.25-1.40, 1.30-1.40, or 1.10-1.35, 1.10-1.30, 1.10-1.25, 1.10-1,20 on the non-Tetranychid arthropod prey, preferably on immobilized Astigmatid prey having immobilized life stages comprising immobilized eggs,
wherein female individuals have predatory behavior towards individuals of a Tetranychid species, preferably predatory behavior characterized by a daily oviposition rate of at least 10, preferably at least 15, more preferably at least 19 eggs per female per 5 days, and/or
wherein the population has an increased reproduction rate as compared to a control Phytoseiulus predatory population of the same species reared on Tetranychid prey as the sole food source.

60. Predatory mite population according to claim 58(ii),
wherein the population is characterized by a juvenile and/or female survival rate of at least 40% on the non-Tetranychid prey,
wherein at least 10% of female individuals of the population are characterized by capability to produce female offspring in a number of subsequent generations, wherein the number of subsequent generations is at least 1, such as at least 2, such as in at least 3, 4, 5, 6, 7, 8, 9 at least 10 generations,
wherein the population is characterized by a daily reproduction rate in the range of about 1.10-1.40, such as 1.15-1.40, 1.20-1.40, 1.25-1.40, 1.30-1.40, or 1.10-1.35, 1.10-1.30, 1.10-1.25, 1.10-1.20, and/or
wherein at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of female individuals of the population are capable of reproducing on a non-tetranychid arthropod prey, preferably on an immobilized non-tetranychid arthropod prey, such as on a non-phytophagous prey, preferably on an Astigmatid prey, most preferably on an immobilized Astigmatid prey, such as an immobilized. Astigmatid prey having immobilized life stages comprising immobilized eggs.

61. Phytoseiulus persimilis predatory mite population according to claim 58(iii), wherein the improved reproduction on a non-tetranychid arthropod prey is characterized by at least one of: increased daily reproduction rate, increased daily oviposition rate, increased survival rate, it creased percentage of female individuals reproducing on said prey, and improved predatory behavior towards a Tetranychidae.

62. Predatory mite population according to claim 58, wherein. the reproduction on a non-tertranychid preyis reproduction on an Astigmatid mite species selected from:
i) Carpoglyphidae such as from the genus Carpoglyphus e.g. Carpoglyphus lactis;
ii) Pyroglyphidae such as from the genus Dermatophagoides e.g. Dermatophagoides pteronysinus, Dermatophagoides farinae from the genus Euroglyphus e.g. Euroglyphus longior, Euroglyphus maynei, from the genus Pyroglyphus e.g. Pyroglyphus africanus;
ii) Glycyphagidae such as from the subfamily Ctenoglyphinae, such as from the genus Diamesoglyphus e.g. Diamesoglyphus intermediusor from the genus Ctenoglyphus, Ctenoglyphus plumiger, Ctenoglyphus canestrinii, Ctenoglyphus palmifer; the subfamily Glycyphaginae, such as from the genus Blomia, e.g. Blomia freemani or from the genus Glycyphagus, e.g. Glycyphagus ornatus, Glycyphagus bicaudatus, Glycyphagus privatus, Glycyphagus domesticus, or from the genus Lepidoglyphus e.g. Lepidoglyphus michaeli, Lepidoglyphus fustifer, Lepidoglyphus destructor, or from the genus Austroglycyphagus, Austroglycyphagus geniculatus; from the subfamily Aëroglyphinae, such as from the genus Aërogiyphus, e.g. Aëroglyphus robustus from the subfamily Labidophorinae, such as from the genus Gohieria, e.g. Gohieria fusca; or from the subfamily Nycteriglyphinae such as from the genus Coprogiyphus, e.g. Coprogiyphus stammeri or from the subfamily Chortoglyphidae, such as the genus Chortoglyphus e.g. Chortoglyphus arcuatus and more preferably is selected from the subfamily Glycyphaginae, more preferably is selected from the genus Glycyphagus or the genus Lepidoglyphus most preferably selected from Glycyphagus domesticus or Lepidoglyphus destructor;
iv) Acaridae such as from the genus Tyrophagus e.g. Tyrophagus putrescentiae, Tyrophagus tropicus, from the genus Acarus e.g. Acarus siro, Acarus farris, Acarus gracilis, from the genus Lardoglyphus e.g, Lardoglyphus konoi, from the genus Thyreophagus, such as Thyreophagus entomophagus, from the genus Aleuroglyphus, e.g. Aleuroglyphus ovatus;
v) Suidasiidae such as from the genus Suidasia, such as Suidasia nesbiti, Suidasia pontifica or Suidasia medanensis.

63. Mite composition comprising a predatory mite population according to claim 58 together with a carrier material, such as a carrier material selected from sawdust, wheat bran, buckwheat husks, rice husks or millet husks, or comprising a mixture thereof, preferably a carrier having carrier elements comprising mite shelters, preferably, further comprising a food source for the Phytoseiulus predatory individuals, wherein the food source comprises a non-Tetranychid arthropod prey, preferably an immobilized non-tetranychid arthropod prey, such as a non-phytophagous prey, preferably an Astigmatid prey, most preferably an immobilized Astigmatid prey, such as an immobilized Astigmatid prey, in particular a Carpogiuhus species, having immobilized life stages comprising immobilized eggs.

64. Device for releasing individuals of a Phytoseiulus predatory mite species, said device comprising a container holding a predatory mite population according to claim 58, preferably in a composition, wherein the device comprises an exit for mobile life stages of the Phytoseiulus predatory mite species, preferably an exits suitable for providing a sustained release of a number of mobile life stages.

65. Method for rearing Phytoseiulus predatory individuals, said method comprising providing a predatory mite population according to claim 58, preferably in a composition, and allowing the Phytoseiulus predatory individuals to prey on the non-Tetranychid arthropod prey.

66. Method for obtaining a predatory mite population according to claim 58, said method comprising the steps of:
(a) providing a rearing population of a predatory mite species selected from the genus Phytoseiulus, said rearing population comprising individuals of the Phytoseiulus species preferably together with a suitable food source for the Phytoseiulus individuals, said food source comprising a prey species selected from the Tetranychidae, (b) providing a preselected non-Tetranychid arthropod species, preferably an Astigmatid mite species, most preferably an immobilized Astigmatid mite species having mobilized life stages comprising immobilized eggs;
(c) providing a the preselected non-Tetranychid arthropod species to the Phytoseiulus individuals as a food source;
(d) selecting Phytoseiulus individuals that are capable of reproduction while using the preselected non-Tetranychid arthropod individuals as a food source;
(e) rearing selected Phytoseiuius individuals on a food source comprising the preselected non-Tetranychid arthropod species;
(f) optionally, alternatingly rearing selected Phytoseiulus individuals in a sequence of:
rearing for at least 2, such as between. 5 and 50, generations while using a food source comprising the preselected non-Tetranychid arthropod species;
rearing for at least 2, such as between 5 and 50, generations while using a food source comprising the prey species selected from the Tetranychidae.

67. The method according to claim 66, wherein the method further comprises steps of
a. separating eggs from the preselected non-Tetranychid arthropod species; b. mixing the separated eggs with a carrier material, such as a carrier material selected from sawdust, wheat bran, buckwheat husks, rice husks or millet husks, or comprising a mixture thereof and water, so as to coat the carrier material with layer of eggs;
c. freezing the mixture; and
d. rearing the Phytoseiulus individuals on the mixture as a food source.

68. Method according to claim 66,
wherein the provided rearing population is a population composed of a number of sub-populations, wherein said sub-populations are from distinct sources, such as from distinct production populations and/or from natural populations isolated. from distinct geographical locations, or
wherein the provided rearing population comprises at least 100 individuals, such as between. 2.00 and 5000 individuals, preferably between 500 and 1500 individuals.

69. Method for obtaining predatory mite population capable of reproduction on a nnetertranychid arthropod prey, preferably on an immobilized non-tetranychid arthropod prey, such as on a non-phytophagous prey, preferably on an Astigmatid prey, most preferably on an immobilized Astigmatid prey, such as an immobilized Astigmatid prey having immobilized life stages comprising immobilized eggs, said method comprising the steps of:
a. providing a rearing population of a predatory mite species selected from the genus Phytosejulus, said rearing population comprising individuals of the Phytoseiulus species, reared on a suitable food source for the Phytoseiulus individuals, said food source comprising a prey species selected from the Tetranychidae species of the genus Phytoseiulus;
b. providing a population of individuals of preselected non-Tetranychid arthropod species, preferably an Astigmatid mite species, most preferably an immobilized Astigmatid mite species having immobilized life stages comprising immobilized eggs;
c. rearing Phytoseiulus individuals on the preselected non-Tetranychid arthropod species as a food source.

70. Method according to claim 69, further comprising steps of:
d. selecting Phytoseiulus individuals that are capable of reproduction while using the preselected non-Tetranychid arthropod individuals as a food source;
e. rearing selected Phytoseiulus individuals on a food source comprising the preselected non-Tetranychid arthropod species;
f. optionally, alternatingly rearing selected Phytoseiulus individuals in a sequence of:
rearing for at least 2, such as between 5 and 50, generations while using a food source comprising the preselected non-Tetranychid arthropod species; rearing for at least 2, such as between 5 and 50, generations while using a food source comprising the prey-species selected from the Tetranychidae.

71. Mite composition comprising a predatory mite population according to claim 58 together with an immobilized non-Tetranychid arthropod prey, preferably an immobilized non-Tetranychid arthropod prey comprising immobilized eggs, such as an immobilized Astigmatid mite species having immobilized life stages comprising frozen. eggs, wherein the eggs are coated with a carrier material, such as a carrier material selected from sawdust, wheat bran, buckwheat husks, rice husks or millet husks, or comprising a mixture thereof, preferably a carrier having carrier elements comprising mite shelters, or wherein the carrier material, such as a carrier material selected from sawdust, wheat bran, buckwheat husks, rice husks or millet husks, or comprising a mixture thereof, preferably a carrier having carrier elements comprising mite shelters is coated by the immobilized non-Tetranychid arthropod prey.

72. Device for releasing individuals of a Phytoseiulus predatory mite species, said device comprising a container holding the composition. according to claim 71, wherein the container comprises an exit for mobile life stages of the Phytoseiulus predatory mite species, preferably an. exits suitable for providing a sustained release of a number of mobile life stages.

73. A biological control composition wherein, the composition comprises
a. a predatory mite population comprising individuals of at least one mite species of the genus Phytoseiulus capable of reproduction on a non-tetranychid arthropod prey, preferably on an immobilized non-tetranychid arthropod prey, such as on a non-phytophaqous prey, preferably on an Astigmatid prey, most: preferably on an immobilized Astigmatid prey, such as an immobilized Astigmatid prey, in particular a Carpogiyhus prey, having immobilized. life stages comprising immobilized eggs; and
b. a prey mite population comprising individuals of a non-tetranychid arthropod prey, preferably an immobilized non-tetranychid arthropod. prey, such as a non-phytophagous prey, preferably an Astigmatid prey, most preferably an immobilized. Astigmatid prey, such as an immobilized Astigmatid prey, in particular a Carpoglyhus prey, having immobilized life stages comprising immobilized eggs, and
c. optionally a carrier, such as a carrier material selected from sawdust, wheat bran, buckwheat husks, rice husks or millet husks, or comprising a mixture thereof, preferably a carrier having carrier elements comprising mite shelters, or
ii) comprises Phytoseiulus predatory individuals, wherein the population is characterized by a daily oviposition rate of at least 0.50, such as >0.55, >0.60, >0.65, >0.70, >0.75, >0.80, >0.90, >0.95, >1.00, >1.05, >1.10, >1.15, >1.20, >1.25, >1.30, >1.35, >1.40, >1.45, >1.50, >1.55, >1.60, >1.65, >1.70, >1.75, >1.80, >1.85, >1.90, >1.95, or at least 2.00 eggs/day/female while preying on non-tetranychid arthropod prey, preferably on an immobilized non-tetranychid arthropod prey, such as on a non-phytophagous prey, preferably on an Astigmatid prey, most preferably on an immobilized Astigmatid prey, such as an immobilized Astigmatid prey, in particular a Carpoglyphus prey, having immobilized life stages comprising immobilized. eggs, or
iii) comprises Phytoseiulus predatory individuals, wherein the population is characterized by improved reproduction on non-tetranychid arthropod prey, preferably on an immobilized non-tetranychid arthropod prey, such as on a non-phytophaqous prey, preferably on an Astigmatid prey, most: preferably on an immobilized Astigmatid prey, such as an immobilized Astigmatid prey, in particular a Carpoglyphus prey, having immobilized life stages comprising immobilized eggs, as compared to a control Phytoseiulus predatory population of the same species, reared on Tetranychid prey as the sole food source.

74. The biological control composition according to claim 73(i),
wherein at least 10% of female individuals of the population is capable of reproduction on a non-tetrahychid arthropod prey, preferably on an immobilized non-tetranychid arthropod prey, such as on a non-phytophagous prey, preferably on an Astigmatid prey, most preferably on an immobilized Astigmatid prey, such as an immobilized Astiqmatid prey, in particular a Carpoglyhus prey, having immobilized life stages comprising immobilized eggs,
wherein at least 15%, at least 20%, at least 25%, at least 30%, at least 35%, at least 40%, at least 45%, at least 50%, at least 55%, at least 60%, at least. 65%, at least 70%, at least 75%, at least 80%, at least 85%, at least 90%, at least 95%, or at least 99% of female individuals of the population are capable of reproducing on a non-tetranychid arthropod prey, preferably on an immobilized non-tetranychid arthropod prey, such as on a non-phytophagous prey, preferably on an Astigmatid prey, most preferably on an immobilized Astigmatid prey, such as an immobilized. Astigmatid prey, in particular a Carpoglyhus prey, having immobilized. life stages comprising immobilized eggs,
wherein the at least 10% of female individuals of the population are capable of oviposition on immobilized Astigmatid prey having immobilized life stages comprising immobilized eggs, wherein the population has a daily oviposition rate of at least:
at least 0.50, such as >0.55, >0.60, >0.65, >0.70, >0.75, >0.80, >0.90, >0.95, >1.00, >1.05, >1.10, >1.15, >1.20, >1.25, >1.30, >1.35, >1.40, >1.45, >1.50, >1.55, >1.60, >1.65, >1.70, >1.75, >1.80, >1.85, >1.90, >1.95, or at least 2.00 eggs/day/female,
wherein the population has a daily oviposition rate of at least at least 0.50, such as >0.55, >0.60, >0.65, >0.70, >0.75, >0.80, >0.90, >0.95, >1.00, >1.05, >1.10, >1.15, >1.20, >1.25, >1.30, >1.35, >1.40, >1.45, >1.50, >1.55, >1.60, >1.65, >1.70, >1.75, >1.80, >1.35, >1.90, >1.95, or at least 2.00 eggs/day/female, when. using the non-tetranychid arthropod prey as the sole food source,
wherein the at least 10% of female individuals are capable of completing a full ontogenetic cycle on the non-Tetranychid arthropod prey, when using the non-tetranychid arthropod prey as the sole food source,
wherein the population is characterized by juvenile and/or female survival rate of at least 40%, preferably at least 45%, 50%, 55%, 60%, 65%, 70%, 75%, 80%, 85%, 90%, or at least 95% on the non-Trtranychid prey,
wherein. the at least 10% of female individuals of the population are characterized by capability to produce female offspring in a number of subsequent generations, wherein the number of subsequent generations is at least 1, such as at least 2, such as in at least 3, 4, 5, 6, 7, 8, 9 at. least 10 generations,
wherein. the population is characterized by a daily reproduction rate in the range of about 1.10-1.40, such as 1.15-1.40, 1.20-1.40, 1.25-1.40, 1.30-1.40, or 1.10-1.35, 1.10-1.30, 1.10-1.25, 1.10-1.20 on the non-Tetranychid prey,
wherein female individuals have predatory behavior towards individuals of a Tetranychid species, preferably predatory behavior characterized by a daily reproduction rate of at least 10, preferably at least 15, more preferably at least 19 eggs per female per 5 days, and/or
wherein the population has an increased reproduction rate as compared to a control Phytoseiulus predatory population of the same species reared on Tetranychid prey as the sole food source.

75. The biological control composition according to claim. 73(ii),
wherein the population is characterized by a juvenile and/or female survival rate of at least 40%, preferably at least 45%, 50%, 55%, 60%, 65%, 70%, 75%, 80%, 85%, 90%, or at least 95% on the non-Tetranychid prey,
wherein the at least 10% of female individuals of the population are characterized. by capability to produce female offspring in a number of subsequent generations, wherein the number of subsequent generations is at least 1, such as at least 2, such as in at least 3, 4, 5, 6, 7, 8, 9 at least 10 generations,
wherein the population. is characterized by a daily reproduction rate in the range of about 1.10-1.40, such as 1.15-1.40, 1.20-1.40, 1.25-1.40, 1.30-1.40, or 1.10-1.35, 1.10-1.30, 1.10-1.25, 1.10-1.20, and/or
wherein at least 15%, at least 20%, at least 25%, at least 30%, at least 35%, at least. 40%, at least 45%, at least 50%, at least 55%, at least 60%, at least 65%, at least 70%, at least 75%, at least 80%, at. least 85%, at least 90%, at least 95%, or at least 99% of female individuals of the population are capable of reproducing on a non-tetranychid arthropod prey, preferably on an immobilized non-fetranychid arthropod prey, such as on a non-phytophagous prey, preferably on an Astigmatid prey, most preferably on an immobilized. Astigmatid prey, such as an immobilized Astigmatid prey, in particular a Carpoglyphus prey, having immobilized life stages comprising immobilized eggs.

76. The biological control composition according to claim 73, wherein the reproduction on a non-tertranychid prey is reproduction on an Astigmatid mite species selected. from:
i) Carpoglyphidae such as from the genus Carpoglyphus Carpoglyphus lactic;
ii) Pyroglyphidae such as from the genus Dermatophagoides e.g. Dermatophagoides pterony sinus, Dermatophagoides farinac, from the genus Euroglyphus e.g. Euroglyphus longior, Euroglyphus maynei, from the genus Pyroglyphus e.g. Pyroglyphus africanus;
iii) Glycyphagidae such as from the subfamily Ctenoglyphinae, such as from the genus Diamesoglyphus e.g. Diamesoglyphus intermediusor from the genus Ctenoglyphus, e.g. Ctenoglyphus plumiger, ctenoglyphus canestrinii, Ctenoglyphus palrnifer; the subfamily Glycyphaginae, such as from the genus Blomia, e.g. Blomia freemani or from the genus Glycyphagus, e.g. Glycyphagus ornatus, Glycyphagus bicaudatus, Glycyphagus privatus, Glycyphagus domesticus, from the genus Lepidoglyphus e.g. Lepidoglyphus michaeli, Lepidoglyphus fustifer, Lepidoglyphus destructor, or from the genus Austroglycyphagus, e.g. Austroglycyphagus geniculatus, from the subfamily Aeroglyphinae, such as from the genus Aeroglyphus, e.g. Aeroglyphus robustus from the subfamily, abidophorinae, such as from the genus Gohieria, Gohieria fusca; or from the subfamily Nycteriglyphinae such as from the genus Coproglyphus, e.g. -Coproglyphus stammeri or from the subfamily Chortoglyphidae, such as the genus Chortoglyphus e.g. Chortoglyphus arcuatus and more preferably is selected from the subfamily Glycyphaginae, more preferably is selected from the genus Glycyphagus or the genus Lepidoglyphus most preferably selected from Glycyphaqus domesticus or Lepidoglyphus destructor,
iv) Acaridae such as from the genus Tyrophagus e.g. Tyrophagus putrescentiae, Tyrophagus tropicus, from the genus Acarus e.g. Acarus siro, Acarus farris, Acarus gracilis, from the genus Lardoglyphus e.g. Lardoglyphus konoi, from the genus Thyreophagus, such as Thyreophacrus entomophagus, from the genus Aleuroglyphus, e.g. Aleuroglyphus ovatus;
v) Suidasiidae such as from the genus Suidasia, such as Suidasia resbiti, Suidasia pontifica or Suidasia medanensis.

77. The Phytoseiulus persimilis predatory mite population according to any one of claim 58, or the composition or the biological control composition thereof, wherein the immobilized Astigmatid prey is selected from the group consisting of immobilized mites, non viable mites, non-hatching eggs, non-viable eggs and a combination thereof.
